(19) United States
(12) Patent Application Publication
(10) Pub. No.: US 2004/0176655 A1
(43) Pub. Date: Sep. 9, 2004

(54) Methods of preparing branched alkyl aromatic hydrocarbons

(76) Inventors: Paul Marie Ayoub, Amstelveen (NL); Steven C. Sumrow, Katy, TX (US); Henk Dirkzwager, Heemstede (NL)

Correspondence Address: Donald F. Haas, Shell Oil Company, Legal - Intellectual Property, P.O. Box 2463, Houston, TX, 77252-2463, US

(21) Appl. No.: 10/772,023

(22) Filed: Feb. 4, 2004

Related U.S. Application Data
(60) Provisional application No. 60/445,296, filed on Feb. 5, 2003.

(51) Int. Cl. C07C 1/00; C07C 2/00

(52) U.S. Cl. 585/324

(57) ABSTRACT

Systems and methods to produce branched alkyl aromatic hydrocarbons are described. Systems may include an olefin isomerization unit, an olefin dimerization unit, a dehydrogenation-isomerization unit, an alkylation unit, a dehydrogenation unit, a hydrogenation unit and/or combinations thereof. Methods for producing branched alkyl aromatic hydrocarbons may include isomerization of olefins in a process stream. The isomerized olefins may be used to alkylate aromatic hydrocarbons. After alkylation of the aromatic hydrocarbons, unreacted components from the alkylation process may be separated from the alkyl aromatic hydrocarbons. The unreacted components from the alkylation process may be recycled back into a process stream or sent to other processing units.

### BACKGROUND OF THE INVENTION

1. Field of Invention

The present invention generally relates to systems and methods for preparing alkyl aromatic hydrocarbons. More particularly, embodiments described herein relate to systems and methods for preparing branched alkyl aromatic hydrocarbons.

2. Description of Related Art

Alkylated aromatic hydrocarbons are important compounds that may be used in a variety of applications or converted to other chemical compounds (e.g. surfactants, sulfonates). Surfactants may be used in a variety of applications such as detergents, soaps and oil recovery.

The structural composition of the alkyl aromatic hydrocarbon may influence the properties of (e.g., water solubility, biodegradability, cold water detergency) of the surfactant and/or detergent produced from the alkyl aromatic hydrocarbon. For example, water solubility may be affected by the linearity of the alkyl group. As the linearity of the alkyl group increases, the hydrophilicity (i.e., affinity for water) of the alkyl aromatic surfactant may decrease. Thus, the water solubility and/or detergency (performance) of the alkyl aromatic surfactant may decrease. Incorporating branches that contain a minimum number of quaternary and/or tertiary carbon atoms into the alkyl portion of the alkyl aromatic surfactant may increase the cold-water solubility and/or detergency of the alkyl aromatic surfactant while maintaining the biodegradability of the detergents. The amount and type of branching of the alkyl group, however, may decrease the biodegradation rate of the surfactant and/or detergent.

Branched alkyl aromatic hydrocarbons are composed of a branched alkyl group coupled to an aromatic group. Branched alkyl groups are composed of a linear alkyl groups with branches extending form the linear alkyl group. Branches of the linear alkyl groups may include one or more aliphatic alkyl groups, a linear alkyl group or combinations thereof. Branches may include methyl, ethyl, propyl or higher alkyl groups. Quaternary and tertiary carbons may be present in a branched the alkyl group. The number of quaternary and tertiary carbons may result from the branching pattern in the alkyl group. As used herein, the phrase “aliphatic quaternary carbon atom” refers a carbon atom that is not bound to any hydrogen atoms and not attached to an aromatic ring system.

A surfactant with a branched alkyl group including quaternary and/or tertiary carbons may have a lower biodegradation rate than a surfactant with a linear or mono-branched alkyl group. As used herein, “biodegradable” refers to a material that can be chemically altered or broken down by bacteria or other natural agents. For example, in a biodegradation experiment using a porous pot activated sludge treatment, a biodegradation rate of sodium 2-methyl-2-undecyl[¹⁴C]benzensulfonate was greater than a biodegradation rate of sodium 5-methyl-5-undecyl[¹⁴C]benzensulfonate. A detailed description of the experiment is described by Nielsen et al. in “Biodegradation of Coproducts of Commercial Linear Alkylbenzene Sulfonate,” Environmental Science and Technology, 1997, 31:3397-3404.

Examples of compositions and process for the manufacture of branched alkyl aromatic hydrocarbons are described in U.S. Pat. No. 3,484,498 to Berg, entitled “Process For The Preparation Of Aryl-Substituted Normal Paraffin Hydrocarbons;” U.S. Pat. No. 5,196,624 to Threlkel et al., entitled “Detergent Grade to C₁₀ to C₂₈ Olefins, (C₁₀ to C₂₈ Alkyl)Benzenes and C₁₀ to C₂₈ Alkyl)Benzene Sulfonates and Process for Preparing Same Using A Phosphite Containing Catalyst;” U.S. Pat. No. 5,196,625 to Threlkel et al. entitled “Detergent Grade to C₁₀ to C₂₈ Olefins, (C₁₀ to C₂₈ Alkyl)Benzenes and C₁₀ to C₂₈ Alkyl)Benzene Sulfonates and Process for Preparing Same Using A Phosphite Containing Catalyst;” U.S. Pat. No. 6,111,158 to Marinangeli et al., entitled “Process For Producing Arylalkanes At Alkylation Conditions Using A Zeolite Having a NES Zeolite Structure Type; and” and U.S. Pat. No. 6,187,981 to Marinangeli et al., entitled “Process For Producing Arylalkanes And Arylalkane Sulfonates, Compositions Produced Therefrom, and Uses Thereof;” all of which are herein incorporated by reference as if fully set forth herein.

### SUMMARY

In an embodiment, alkyl aromatic hydrocarbons may be produced by a method that includes isomerizing olefins in an isomerization unit. The isomerized olefins may be used to alkylate aromatic hydrocarbons. After alkylation of the aromatic hydrocarbons, unreacted components from the alkylation process may be separated from the produced alkyl aromatic hydrocarbon products. Paraffins in the separated stream may be subjected to a dehydrogenation process where paraffins in the separated stream may be dehydrogenated to generate additional olefinic components. At least a portion of the resulting olefinic stream from the dehydrogenation process may be recycled back into the isomerization unit.

Isomerization of olefins in a process stream may occur in an isomerization unit. In an embodiment, a process feed stream entering an isomerization unit may include linear olefins and paraffins having an average carbon number from 7 to 16. In an embodiment, a process feed stream entering an isomerization unit includes linear olefins and paraffins having an average carbon number from 10 to 13. As used herein, the phrase “carbon number”-refers to the total number of carbons in a molecule. In certain embodiments, a process feed stream entering an isomerization unit is derived from a Fischer-Tropsch process. At least a portion of the linear olefins in a hydrocarbon stream may be isomerized to branched olefins in the isomerization unit. Branched olefins may have an average number of branches per olefin molecule of between about 0.7 and about 2.5. Branched olefins may include, but are not limited to, methyl and/or ethyl branches. The isomerization process may produce branched olefins that include less-than about 0.5 percent of aliphatic quaternary carbon atoms.

In an embodiment, one or more hydrocarbons streams may be combined with the feed stream entering the isomerization unit. The hydrocarbons streams may be mixed with the feed stream to alter the concentration of the olefins entering the isomerization unit. After the feed stream is processed in the isomerization unit, the resulting branched olefin containing stream is passed into an alkylation unit. One or more hydrocarbon streams may be combined with the branched olefin containing stream to alter the concentration of olefins entering the alkylation unit.

Alkylation of aromatic hydrocarbons with olefins may occur in an alkylation unit. In an embodiment, an olefinic hydrocarbons stream from an isomerization unit and aromatic hydrocarbons may enter an alkylation unit. In the alkylation unit, at least a portion of the aromatic hydrocarbons may be alkylated with at least a portion of the olefins in the hydrocarbon stream to produce alkyl aromatic hydrocarbons. At least a portion of the produced alkyl aromatic hydrocarbons may include a branched alkyl group. At least a portion of the unreacted components of the hydrocarbon stream, at least a portion of unreacted aromatic hydrocarbons and at least a portion of the produced alkyl aromatic hydrocarbons form an alkylation reaction stream.

At least a portion of the paraffins, unreacted olefins, aromatic hydrocarbons and alkyl aromatic hydrocarbons from the alkylation reaction stream may be separated to produce an unreacted hydrocarbons stream and an alkyl aromatic hydrocarbons product stream. The unreacted hydrocarbons stream may be further separated, in some embodiments, to form a paraffins and unreacted olefins stream and an aromatic hydrocarbons stream. At least a portion of the unreacted aromatic hydrocarbons stream may be recycled to the alkylation unit.

Dehydrogenation of paraffins may occur in a dehydrogenation unit. In an embodiment, at least a portion of a paraffins and unreacted olefins stream may enter a dehydrogenation unit. In the dehydrogenation unit, at least a portion of the paraffins in the paraffins and unreacted olefins stream may be dehydrogenated to produce olefins. At least a portion of the produced olefins may exit the dehydrogenation unit to form an olefinic hydrocarbon stream. The resulting olefinic hydrocarbon stream from the dehydrogenation process may be recycled back into the isomerization unit and/or into a stream entering the isomerization unit.

In an embodiment, a feed stream that includes olefins and paraffins may be processed in a dehydrogenation-isomerization unit. The process feed stream entering a dehydrogenation-isomerization unit, in some embodiments, is derived from a Fischer-Tropsch process. In the dehydrogenation-isomerization unit at least a portion of the paraffins in the feed stream may be dehydrogenated to form olefins. The dehydrogenation-isomerization unit may also isomerize at least a portion of the resulting olefins and at least a portion of the olefins that were already present in the feed stream. The isomerization process converts linear olefins (e.g., unbranched olefins) into branched olefins.

Process conditions in the dehydrogenation-isomerization unit may be such that the resulting branched olefins have an average number of branches per olefin molecule of between about 0.7 and about 2.5. The branched olefin may include, but is not limited to methyl and/or ethyl branches. The isomerization process may produce branched olefins that include less than about 0.5 percent of quaternary aliphatic carbon atoms. Process conditions in the dehydrogenation-isomerization unit may include a catalyst that has two functions, one to dehydrogenate the paraffins to olefins and the second to isomerize the olefins.

In an embodiment, the dehydrogenation-isomerization unit may include a plurality of zones. The plurality of zones may include a first reaction zone and a second reaction zone. The first reaction zone may be a dehydrogenation zone. The second reaction zone may be an isomerization zone. A hydrocarbon stream, containing olefins and paraffins, may enter the dehydrogenation zone. At least a portion of the paraffins in the hydrocarbon stream may be dehydrogenated to olefins to produce a stream enriched in olefins. The enriched olefin stream may be passed into the isomerization zone. In the isomerization zone, at least a portion of the olefins in the enriched olefin stream may be isomerized to branched olefins. The branched olefins may be used to alkylate aromatic hydrocarbons. An alkylation unit alkylates at least a portion of aromatic hydrocarbons with at least a portion of the olefins in the combined stream. After alkylation of the aromatic hydrocarbons, paraffins and unreacted olefins and aromatic hydrocarbons from the alkylation process may be separated from the alkyl aromatic hydrocarbon products. The paraffins and unreacted olefins may be separated from the aromatic hydrocarbons to form a paraffins and unreacted olefins stream. The paraffins and unreacted olefins stream may recycle by directing the paraffins and unreacted olefins stream back into the dehydrogenation-isomerization unit and/or into a stream entering the dehydrogenation-isomerization unit. The aromatic hydrocarbons may be recycled back to the alkylation unit.

In certain embodiments, a feed stream is fed into a dimerization unit that produces dimerized olefins. The produced dimerized olefins may include branched dimerized olefins. A process feed stream entering a dimerization unit is derived, in some embodiments, from a Fischer-Tropsch process. In an embodiment, produced dimerized olefins may be separated from the feed stream. The separated feed stream, in some embodiments, may be introduced into the dimerization unit. The produced dimerized olefins may be used to alkylate aromatic hydrocarbons. After alkylation of the aromatic hydrocarbons, at least a portion of unreacted components from the alkylation process may be separated from the alkyl aromatic hydrocarbon products.

In another embodiment, an alkyl aromatic hydrocarbon may be produced by a method that includes producing olefins in a dimerization unit. The produced dimerized olefins may include branched dimerized olefins. The produced dimerized olefins may be used to alkylate aromatic hydrocarbons. An isomerization unit may also be used to produce-branched olefins as described above. In an embodiment, at least a portion of the product stream exiting the dimerization unit may be combined with at least a portion of the product stream exiting the isomerization unit and the combined stream directed to an alkylation unit. The alkylation unit alkylates aromatic hydrocarbons with at least a portion of the olefins in the combined stream. After alkylation of the aromatic hydrocarbons, at least a portion of unreacted components from the alkylation process may be separated from the alkyl aromatic hydrocarbon products.

Process conditions in the dimerization unit may be such that the resulting branched olefins have an average number of branches per olefin molecule of between about 0.7 and about 2.5. The branched olefin may include, but is not limited to methyl and/or ethyl branches. The isomerization process may produce branched olefins that include less than about 0.5 percent of quaternary carbon atoms. In an embodiment, the feed stream entering the dimerization unit includes alpha-olefins having an average carbon number from 4 to 8. The branched olefins produced from the dimerization of alpha-olefins having an average carbon number from 4 to 8 will have an average carbon number from 8 to 16.

At least a portion of the unreacted components and the produced dimerized olefins may be separated to produce an unreacted hydrocarbon stream and a produced dimerized olefins stream. At least a portion of the unreacted hydrocarbons stream may be recycled to the dimerization unit.

In an embodiment, a feed stream containing olefins and paraffins may be processed in a hydrogenation unit. A process feed stream entering a hydrogenation unit is derived, in some embodiments, from a Fischer-Tropsch process. In the hydrogenation unit at least a portion of the olefins in the feed stream may be hydrogenated to form paraffins. The resulting paraffinic feed stream may be fed into a dehydrogenation unit. At least a portion of the paraffins may be dehydrogenated to form an olefinic hydrocarbons feed stream. The olefinic hydrocarbons feed stream may be processed in an isomerization unit and/or a dimerization unit. At least a portion of the olefins in the olefinic feed stream may be isomerized to branched olefins in the isomerization unit. At least a portion of the olefins in the olefinic feed stream may be dimerized in the dimerization unit. The olefins produced from the dimerization and/or isomerization unit may be used to alkylate aromatic hydrocarbons to form branched alkyl aromatic hydrocarbons.

In an embodiment, a feed stream containing olefins and paraffins may be processed in a hydrogenation unit. A process feed stream entering a hydrogenation unit is derived, in some embodiments, from a Fischer-Tropsch process. In the hydrogenation unit at least a portion of the olefins in the feed stream may be hydrogenated- to form paraffins. The resulting paraffinic feed stream may be fed into a dehydrogenation-isomerization unit. At least a portion of the paraffins in the feed stream may be dehydrogenated to form olefins. The dehydrogenation-isomerization unit may also isomerize at least a portion of the resulting olefins and at least a portion of the olefins that were already present in the feed stream. The olefins produced from the dehydrogenation-isomerization unit may be used to alkylate aromatic hydrocarbons to form branched alkyl aromatic hydrocarbons.

In certain embodiments, at least a portion of the alkyl aromatic hydrocarbon product streams from the above-described processes may be sulfonated to form alkyl aromatic sulfonates. In some embodiments, alkyl aromatic sulfonates may include branched alkyl groups.

### BRIEF DESCRIPTION OF THE DRAWINGS

Advantages of the present invention will become apparent to those skilled in the art with the benefit of the following detailed description of embodiments and upon reference to the accompanying drawings, in which:

FIG. 1 depicts a schematic diagram of an embodiment of a system for producing branched alkyl aromatic hydrocarbons using an olefin isomerization unit.

FIG. 2 illustrates structures of aliphatic and olefin sites as determined by ¹H NMR analysis in an embodiment of a method to produce alkyl aromatic hydrocarbons.

FIG. 3 depicts a schematic diagram of an embodiment of a system for producing branched alkyl aromatic hydrocarbons using an olefin isomerization unit with addition of an additional hydrocarbon stream.

FIG. 4 depicts a schematic diagram of an embodiment of a system for producing branched alkyl aromatic hydrocarbons using a dehydrogenation-isomerization unit.

FIG. 5 depicts a schematic diagram of an embodiment of a system for producing branched alkyl aromatic hydrocarbons using a dehydrogenation-isomerization unit with addition of an additional hydrocarbon stream.

FIGS. 6A-B depict schematic diagrams of embodiments of a system for producing branched alkyl aromatic hydrocarbons using a two-zone dehydrogenation-isomerization unit.

FIG. 7 depicts a schematic diagram of an embodiment of a system for producing branched alkyl aromatic hydrocarbons using a dehydrogenation-isomerization unit with a stacked bed catalyst configuration.

FIG. 8A depicts a schematic diagram of an embodiment of a system for producing branched alkyl aromatic hydrocarbons using a dimerization unit.

FIG. 8B depicts a schematic diagram of an embodiment of a separation unit to separate produced dimerized olefins from a reaction mixture.

FIG. 9 depicts a schematic diagram of an embodiment of a system for producing branched alkyl aromatic hydrocarbons using a dimerization unit and an isomerization unit.

FIG. 10 depicts a schematic diagram of an embodiment of a system for producing alkyl aromatic hydrocarbons using a hydrogenation unit, dehydrogenation unit and an isomerization unit.

FIG. 11 depicts a schematic diagram of an embodiment of a system for producing alkyl aromatic hydrocarbons using a hydrogenation unit and a dehydrogenation-isomerization unit.

FIG. 12A depicts a schematic diagram of an embodiment of a system for producing alkyl aromatic hydrocarbons using a hydrogenation unit, dehydrogenation unit and a dimerization unit.

FIG. 12B depicts a schematic diagram of an embodiment of a separation unit to separate produced dimerized olefins from a reaction mixture.

FIG. 13 depicts a schematic diagram of an embodiment of a system for producing alkyl aromatic hydrocarbons using a hydrogenation unit, dehydrogenation unit, dimerization unit and an isomerization unit.

While the invention is susceptible to various modifications and alternative forms, specific embodiments thereof are shown by way of example in the drawing and will herein be described in detail. It should be understood that the drawings and detailed description thereto are not intended to limit the invention to the particular form disclosed, but on the contrary, the intention is to cover all modifications, equivalents, and alternatives falling within the spirit and scope of the present invention as defined by the appended claims.

### DETAILED DESCRIPTION OF EMBODIMENTS

Hydrocarbon products may be synthesized from synthesis gas (i.e., a mixture of hydrogen and carbon monoxide) using a Fischer-Tropsch process. Synthesis gas may be derived from coal or by reforming of natural gas. The Fischer-Tropsch process catalytically converts synthesis gas into a mixture of products that includes primarily saturated hydrocarbons, a certain amount of olefins and a minor amount of oxygen containing products. The products from a Fischer-Tropsch process may be used for the production of fuels (e.g., gasoline, diesel oil), lubricating oils and waxes. Fuels and other products produced by a Fischer-Tropsch process generally have low levels of sulfur, nitrogen and/or metals. Fischer-Tropsch produced fuels and products also contain little or no cyclic compounds (e.g., aromatics, naphthalenes).

Fischer-Tropsch process streams may also be used to prepare economically valuable commodity products. For example, linear olefins are commodity products that are useful for the production of surfactants. Using a portion of the process stream to produce linear olefins may increase the economic value of a Fischer-Tropsch process stream.

More recently, it has been found that surfactants derived from branched olefins have different, more desirable properties than surfactants derived from linear olefins. For example, surfactants derived from branched olefins have increased water solubility, improved detergency properties and acceptable biodegradable properties compared to surfactants derived from linear olefins. Production of branched olefins from a Fischer-Tropsch process stream may increase an economic value of the stream. In some embodiments, linear olefins may be converted into branched olefins using a isomerization catalyst, a dehydrogenation-isomerization catalyst and/or dimerization catalyst. Branched olefins may be used to produce surfactants that are more desirable and thus more valuable to the producer of the Fischer-Tropsch stream. In general, Fischer-Tropsch processes tend to produce minor amounts of olefins. Increasing an olefin content (e.g. branched alkyl olefin content) of a Fischer-Tropsch stream may increase the economic value of the stream.

Methods are described for increasing the amount of branched olefins derived from a process stream that includes minor amounts of olefins. Increasing the amount of branched olefins in a process stream may increase the economic value of the process stream. Such methods are useful for both Fischer-Tropsch process streams and product streams from other sources that include hydrocarbons.

A hydrocarbon feed stream composition may include paraffins and olefins. At least a portion of the hydrocarbon stream may be made up of linear paraffins and olefins having at least 4 carbon atoms and up to 16 carbon atoms. A hydrocarbon feed stream may be obtained from a Fischer-Tropsch process and/or from an ethylene oligomerization process. A Fischer-Tropsch process may include the conversion of synthesis gas, (i.e., a mixture of carbon monoxide and hydrogen) into hydrocarbons. Fischer-Tropsch catalysts and reaction conditions may be selected to provide a particular mix of products in the reaction product stream. For example, a Fischer-Tropsch catalyst and reaction conditions may be selected to increase the amount of olefins and decrease the amount of paraffins and oxygenates in the stream. Alternatively, the catalyst and reaction conditions may be selected to increase the amount of paraffins and decrease the amount of olefins and oxygenates in the stream.

A catalyst used in a Fischer-Tropsch process may be Mo, W, Group VIII compounds and/or combinations thereof. Group VIII compounds include iron, cobalt, ruthenium, rhodium, platinum, palladium, iridium and osmium. Combinations of Mo, W and Group VIII compounds may be prepared in the free metal form. In an embodiment, combinations of Mo, W and Group VIII compounds may be formed as alloys. Combinations of Mo, W and Group VIII may be formed, in some embodiments, as oxides, carbides or other compounds. In other embodiments, combinations of Mo, W and Group VIII compounds may be formed as a salt. Iron based and cobalt based catalysts have been used commercially as Fischer-Tropsch catalysts. Ruthenium catalysts tend to favor the formation of high melting waxy species under high-pressure conditions. Synthetic Fischer-Tropsch catalysts may include fused iron and contain a promoter such as potassium or oxides on a silica, alumina or silica-alumina support. Cobalt metal may also be used in a Fischer-Tropsch catalyst. With the proper selection of supports, promoters and other metal combinations, a cobalt catalyst may be tuned to manufacture a composition enriched in the desired hydrocarbon species. Other catalysts, such as iron-cobalt alloy catalysts, are known for their selectivity toward the production of olefins. Catalysts or combinations of catalyst that favor the manufacture of desired hydrocarbon species in a Fischer-Tropsch process are generally known.

While reference is made to a Fischer-Tropsch stream, it is to be understood that any stream, made by any process, that includes olefins and saturated hydrocarbons may be a suitable feedstock for the processes disclosed herein. Many Fischer-Tropsch streams may contain from 5 percent to 99 percent olefins, the remainder being saturated hydrocarbons comprising paraffins and other compounds. The Fischer-Tropsch stream may be separated into streams which are composed of a narrow fraction of olefins having an average carbon number from 4 to 8 for processes that use a dimerization unit or, in some embodiments, from 7 to 16 for process that involve an isomerization unit.

In some embodiments, feed streams containing olefins and paraffins are obtained through cracking of paraffin wax and/or the oligomerization of olefins. Commercial olefin products manufactured by ethylene oligomerization are marketed in the United States by Chevron Chemical Company, Shell Chemical Company (under the trademark NEODENE), and by British Petroleum. Cracking of paraffin wax to produce alpha-olefin and paraffin feed streams is described in U.S. Pat. No. 4,579,986 to Sie, entitled “Process For The Preparation Of Hydrocarbons” and U.S. patent application Ser. No. 10/153,955 to Ansorge et al., entitled “Process For The Preparation of linear Olefins and Use Thereof To Prepare Linear Alcohols” both of which are incorporated by reference herein. Specific procedures for preparing linear olefins from ethylene are described in U.S. Pat. No. 3,676,523 to Mason, entitled “Alpha-Olefin Production;” U.S. Pat. No. 3,686,351 to Mason, entitled “Alpha-Olefin Production;” U.S. Pat. No. 3,737,475 to Mason, entitled “Alpha-Olefin Production” and U.S. Pat. No. 4,020,121 to Kister et al., entitled “Oligomerization Reaction System,” all of which are incorporated herein by reference. Most of the above-mentioned processes produce alpha-olefins. Higher linear internal olefins may be commercially produced, for example by the chlorination-dehydrochlorination of paraffins, by paraffin dehydrogenation, or by isomerization of alpha-olefins.

In an embodiment, a feed stream is processed to produce a hydrocarbon stream that includes branched olefins. These branched olefins may be used to alkylate an aromatic hydrocarbon to produce branched alkyl aromatic hydrocarbons. The feed stream may have a paraffin content range between about 50 percent by weight to about 90 percent by weight of the feed stream. In certain embodiments, a feed stream may have a paraffin content greater than about 90 percent by weight paraffins. The feed stream may also include olefins. The olefin content of the feed stream may be between about 10 percent by weight to about 50 percent by weight. In other embodiments, a feed stream may-have an olefin content greater than 90 percent by weight olefins. The composition of the feed stream may include hydrocarbons having an average carbon number ranging from 4 to 30. In an embodiment, an average carbon number of the hydrocarbons in a feed stream may range from 4 to 24. In other embodiments, an average carbon number of the feed stream may range from 4 to 16. The average carbon number of the hydrocarbons may range from 7 to 16 for processes that involve an isomerization unit. In certain embodiments, an average carbon number of the hydrocarbons may range from 10 to 13 for processes that involve an isomerization unit. The average carbon number of the hydrocarbons may range from 4 to 6 for processes that use a dimerization unit. In certain embodiments, an average carbon number of the hydrocarbons range from 4 to 8 for processes that use a dimerization unit. A feed stream may include minor amounts of hydrocarbons having a carbon number that is higher and/or lower then the desired carbon number range. In some embodiments, a feed stream may be derived from distillation of a process stream that includes a broader range of carbon numbers.

In an embodiment, a feed stream for an isomerization unit and/or dimerization unit includes mono-olefins and/or paraffins. The mono-olefins may be of a linear and/or branched structure. The mono-olefins may have an alpha or internal double bond position. The feed stream may include olefins in which 50 percent or more of the olefin molecules present may be alpha-olefins of a linear (straight chain) carbon skeletal structure. In certain embodiments, at least about 70 percent of the olefins are alpha-olefins of a linear carbon skeletal structure. A hydrocarbon stream in which greater than about 70 percent of all of the olefin molecules are alpha-olefins of a linear carbon skeletal structure may be used in certain embodiments of alkylation of aromatic hydrocarbons. Such a stream may be derived from a Fischer-Tropsch process. In some embodiments, a feed stream includes olefins in which at least about 50 percent of the olefin molecules present are internal olefins.

Branched chain olefins may be used as a feed source for an aromatic alkylation reaction. An aromatic alkylation reaction uses olefins in an incoming stream to alkylate aromatic compounds. Aromatic compounds (e.g., aromatic hydrocarbons) may include benzene or substituted benzene. In an embodiment, alkyl-substituted benzene is used as a substituted benzene in the process. Alkyl-substituted benzenes may be mono- and/or poly-substituted lower alkyl benzenes. The alkyl substituent of an alkyl substituted benzene may have a carbon number ranging from 1 to 5. In an embodiment, a carbon number of the alkyl substituent may range from 1 to 2. Suitable examples of aromatic compounds include, but are not limited to, benzene, toluene, xylenes, ethylbenzene, cumene, n-propylbenzene and other mono- and poly-lower alkylbenzenes. In some embodiments, a single aromatic compound and/or a mixture of two or more aromatic compounds may be used as feed source. Aromatic hydrocarbons may be fed into the reactor directly or mixed in a suitable non-reactive organic solvent prior to addition to the alkylation unit.

In certain embodiments, to reduce production costs of producing alkyl aromatic hydrocarbons, a stream containing a significant amount of paraffins and a minor amount of olefins may first be isomerized then alkylated to form alkyl aromatic hydrocarbons. Processing a stream containing a minor amount of olefins through an isomerization unit prior to alkylation may save production time, dehydrogenation catalyst cost, and/or enhance the overall economic viability of the stream. In some embodiments, after alkylation, paraffins and unreacted olefins may be recycled to a dehydrogenation unit to produce a stream enriched in olefins. The enriched olefins stream may be recycled into an isomerization unit.

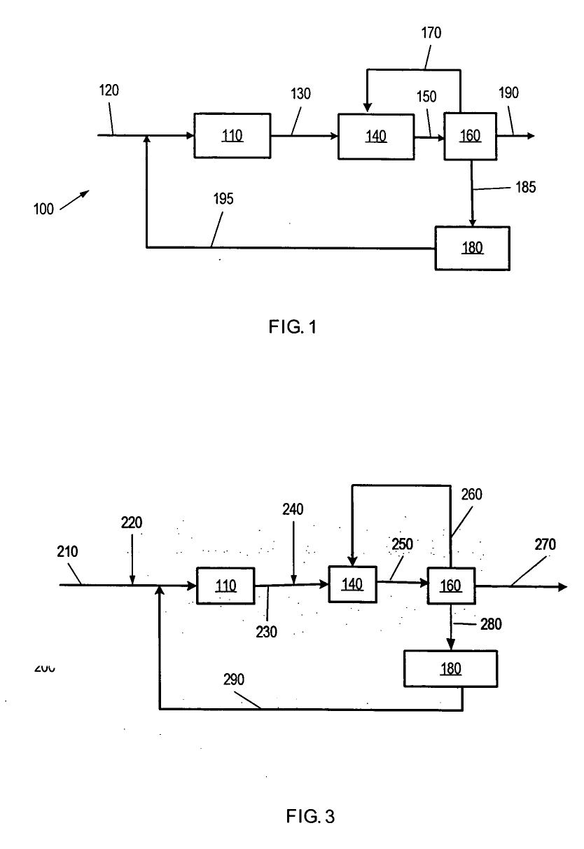

Referring to System 100, depicted in FIG. 1, an embodiment of a method to produce branched alkyl aromatic hydrocarbons is described herein. A first hydrocarbon stream containing olefins and paraffins may enter isomerization unit 110 via first conduit 120. The first hydrocarbon stream may include hydrocarbons with an average carbon number from 7 to 16 or, in some embodiments, from 10 to 13. In some embodiments, a first hydrocarbon stream includes alpha-olefins. In certain embodiments, a first hydrocarbon stream is a stream derived from a Fischer-Tropsch process. The alpha-olefin content of the first hydrocarbon stream may be greater than about 70 percent of the total amount of olefins in the first hydrocarbon stream. In isomerization unit 110, at least a portion of the olefins in the first hydrocarbon stream may be isomerized to branched olefins to produce a second hydrocarbon stream.

In certain embodiments, isomerization unit 110 may have several points of entry to accommodate process streams which may vary in composition. The process streams may be from other processing units and/or storage units. Examples of process streams include, but are not limited to, a diluent hydrocarbon stream, and/or other hydrocarbon streams that include olefins and paraffins derived from other processes. As used herein, “entry into the isomerization unit” refers to entry of process streams into the isomerization unit through one or more entry points.

The conditions for olefin isomerization in isomerization unit 110 may be controlled such that the number of carbon atoms in the olefins prior to and subsequent to the isomerization conditions is substantially the same. U.S. Pat. No. 5,648,584 to Murray, entitled “Process for Isomerizing Linear Olefins to Isoolefins” and U.S. Pat. No. 5,648,585 to Murray et al., entitled “Process for Isomerizing Linear Olefins to Isoolefins” both of which are incorporated herein by reference; describe, in detail, catalysts and process conditions to skeletally isomerize linear olefins to branched olefins.

In an embodiment, linear olefins in a first hydrocarbon stream are isomerized in isomerization unit 110 by contacting at least a portion of the first hydrocarbon stream with a zeolite catalyst. The zeolite catalyst may have at least one channel with a crystallographic free channel diameter ranging from greater than 4.2 Å and less than about 7 Å. The zeolite catalyst may have an elliptical pore size large enough to permit entry of a linear olefin and diffusion, at least partially, of a branched olefin. The pore size of the zeolite catalyst may also be small enough to retard coke formation. As used herein, “coke” refers to the product from thermal degradation of a molecule into smaller molecules.

Temperatures at which the olefin isomerization may be conducted range from about 200° C. to about 500° C. Temperatures in isomerization unit 110 are, in some embodiments, kept below the temperature at which the olefin will crack extensively. As used herein, “cracking” refers to the process of thermally degrading molecules into smaller molecules. To inhibit cracking, low temperatures may be used at low feed rates. In certain embodiments, lower temperatures may be-used when the amount of oxygenates present in the process stream is low. Higher feed rates may be desirable to increase the production rate of isomerised products. Higher feed rates may be used, in some embodiments, when operating at higher reaction temperatures. The reaction temperature, however, should be set such that cracking to lower boiling weight products is minimized. For example, greater than 90 percent of linear olefins may be converted to branched olefins at 230° C. at a feed rate of 60 grams per hour with minimal cracking. Pressures maintained in isomerization unit 110 may be at a hydrocarbon partial pressure ranging from about 0.1 atmosphere (10 kPa) to about 20 atmospheres (2026 kPa). In an embodiment, partial pressure may range from above about 0.5 atmosphere (51 kPa) to about 10 atmospheres (1013 kPa).

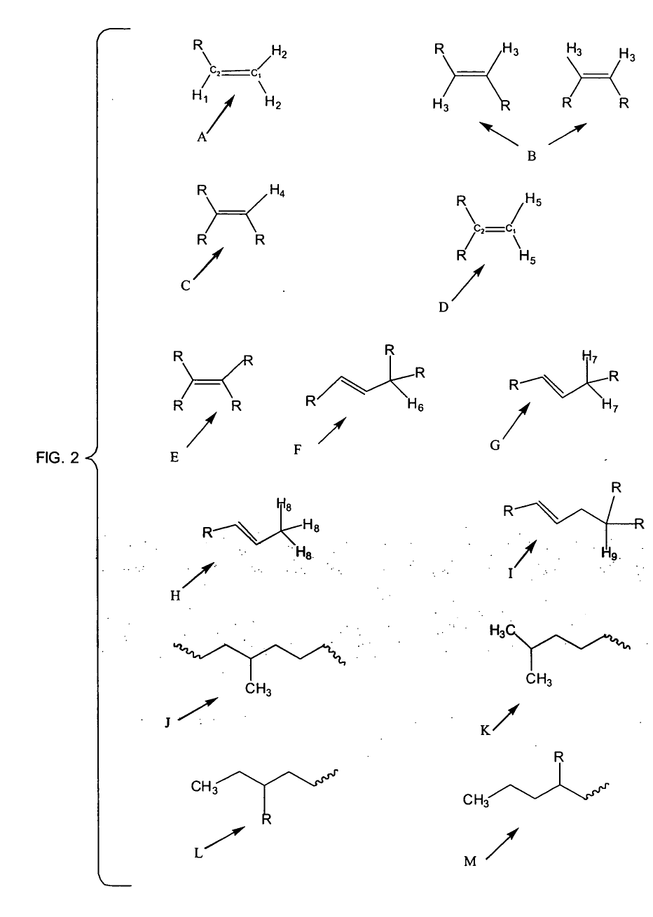

The branched olefin produced in isomerization unit 110 may include methyl, ethyl and/or longer carbon chain branches. Hydrogen Nuclear Magnetic Resonance (¹H NMR) analysis of the isomerized olefin composition may be performed. Branched olefins may include quaternary and/or tertiary aliphatic carbons. In certain embodiments, an amount of quaternary and/or tertiary aliphatic carbons produced in an isomerization unit may be minimized. ¹H NMR analysis of the olefins produced by the isomerization unit may indicate the extent of isomerization of the olefins in the hydrocarbon stream. ¹H NMR method may be capable of differentiating a wide range of olefin structures such as the olefin structures illustrated in FIG. 2.

A ¹H NMR technique may use a combination of a 12-degree tip and a 5 second recycle delay time. For example, a spectral width of 8 KHz on a 500 MHz instrument may be used in the analysis. Enough scans (e.g., 64) may be performed to give adequate signal to noise ratio for the detection of the aliphatic and olefinic sites in the olefin molecules. Aliphatic and olefin sites may be calculated through analysis of the resulting ¹H NMR spectrum. It is assumed in this method that there is one double bond per molecule. The total number of branches is a sum of all aliphatic and olefinic branch-sites per olefin molecule. The average carbon number per molecule may be provided as an input to a carbon 13 (¹³C NMR) calculation using by analytical techniques generally known (e.g. gas chromatography coupled with mass spectrometry). Aliphatic branches, as used herein, are branches on non-olefinic carbons. Olefinic branches, as used herein, are branches on olefinic carbons. The total number of branches on a double bond may be determined by summation of the individual contributions of the various assayed olefin units. Olefin units include vinyl, di-substituted, tri-substituted, vinylidene and/or tetra-substituted olefins, as illustrated in FIG. 2. The amount and type of olefin may vary with process stream composition and isomerization reaction conditions. In an embodiment, an amount of tetra-substituted olefin produced may be low.

As illustrated in FIG. 2, vinyl substituted olefin A is defined as an olefin having one functional group (R) and hydrogen H₁ bound to carbon C₂ and two hydrogens H₂ bound to carbon C₁ of the double bond. A “functional group (R)” as used herein, refers to any aliphatic and/or aromatic group(s) other than hydrogen that can be covalently bound to a carbon atom making up the structure of the double bond. Di-substituted olefin B, as used herein, refers to an olefin having two functional groups R and two hydrogens H₃ covalently bound to each of the carbon atoms of the double bond. Olefin B may be a cis-olefin, a trans-olefin or a mixture thereof. Tri-substituted olefin C, as used herein, refers to an olefin having functional groups R and hydrogen H₄ covalently bound to the carbon atoms of the double bond. Vinylidene olefin D, as used herein, refers to an olefin having two functional groups R covalently bound to carbon atom C₂ and two hydrogens H₅ covalently bound to carbon atom C, of the double bond. Tetra-substituted olefin E, as used herein, refers to an olefin having four functional groups R (i.e., no hydrogens) covalently bound to the carbons of the double bond. Tetra-substituted olefins are not directly detected in the ¹H NMR spectrum since they bear no hydrogens bound to the carbon atoms of the double bond. Tetra-substituted olefins may be determined by calculating the difference between the numbers of all olefin units, as determined from the aliphatic portion of the spectrum adjacent to the double bond (e.g., hydrogens H₆, H₇, and H₈, in Structures F-H of FIG. 2) and the directly identified olefins bearing hydrogens on carbons atoms of the double bond.

For example, in a solution containing olefins A, B, C, D and E, the total number of olefin branches per olefin molecule may be calculated in the following manner. The olefinic branching values may be determined by calculating the average number of branches of the individual contributions of the various assayed olefin units. In the solution, vinyl olefin A and di-substituted olefins B (e.g., cis- and trans-olefins) contribute no olefinic branches. Similarly, tri-substituted olefin C and vinylidene olefin D contribute one olefin branch each. Tetra-substituted olefin E contributes two olefin branches. Therefore, in this example, the total number of olefinic branches would be four and the average olefin branching per molecule would be about 0.67 (4 branches per six olefin molecules).

A total number of branches on aliphatic carbons in the olefin molecules (e.g., structures F and I in FIG. 2) may be determined by summation of the individual contribution of structures with methines adjacent to double bonds (H₆) and structures with methines not adjacent to the double bond (H₉). Olefins F and I would each contribute one branch per olefin molecule, assuming no additional aliphatic branches in the R chains. It is to be understood that, although as shown, olefins F and I are derivatives of olefin B, methine hydrogens may be found in olefins A-E depending on the branching in the R groups.

The presence of quaternary carbon atoms may be determined using (¹³C) NMR analysis. Branching type (e.g., methyl, ethyl, propyl or larger groups) may be determined by hydrogenation of the olefin mixture and then ¹³C NMR analysis of the hydrogenated olefin solution. ¹³C NMR may resolve methyl groups that are directly attached to the hydrogenated olefin backbone structure (e.g., CH₃ in structure J and K of FIG. 2), methyls in ethyl groups attached to the hydrogenated olefin backbone structure (e.g., CH₃ in structure L of FIG. 2), and methyls in propyl or longer groups attached to the hydrogenated olefin backbone structure (e.g., CH₃ in structure M of FIG. 2). Various methyl peak positions in FIG. 2 are given in parts per million (ppm) relative to tetramethyl silane.

Methyls in structure M in FIG. 2 may include terminal methyls, propyl and/or larger branches. The number of propyl and/or larger branches may not be directly obtained from the 13.5-15 ppm peak region in the spectrum. Propyl or larger branched values maybe computed by taking the difference between the total number of branches per molecule and the number of methyl and ethyl branches per molecule obtained directly from the methyl spectral regions of structures J-L in FIG. 2 The total number of branches per molecule is determined by adding up all methyls per molecule minus “two”, wherein “two” accounts for the hydrogenated olefin backbone's terminal methyls.

In an embodiment, an average number of branches per olefin molecule present in the branched olefin composition is between about 0.1 and about 2.5. In other embodiments, an average number of branches per olefin molecule present in the branched olefin composition is between about 0.7 and about 2.5. In certain embodiments, when the feed stream contains greater than 70 percent linear olefins, an average number of branches per olefin molecule present in the branched olefin composition is between about 0.2 and about 1.5. The degree of branching in the product may be controlled by controlling process conditions used in the isomerization unit. For example, high reaction temperatures and lower feed rates may result in a higher degree of branching. Methyl branches may represent between about 20 percent to about 99 percent of the total number of branches present in the olefin molecules. In some embodiments, methyl branches may represent greater than about 50 percent of the total number of branches in the olefin molecules. The number of ethyl branches in the olefin molecules may represent, in certain embodiments, less than about 30 percent of the total number of branches. In other embodiments, a number of ethyl branches, if present, may be between about 0.1 percent and about 2 percent of the total number of branches. Branches other than methyl or ethyl, if present, may be less than about 10 percent of the total number of branches.

The number of aliphatic quaternary and/or tertiary carbon atoms present in the branched olefin composition may be less than about 2 percent of the carbon atoms present. In an embodiment, an aliphatic quaternary and/or tertiary carbons present are less than about 1 percent of the carbon atoms present. For applications in which biodegradability is important, a number of aliphatic quaternary carbon atoms may be less than about 0.5 percent of the carbon atoms present. In an embodiment, a number of aliphatic quaternary and/or tertiary carbon atoms is less than about 0.3 percent of the carbon atoms present. In other embodiments, a number of aliphatic quaternary carbon atoms present in the branched olefin composition is between about 0.01 percent and about 0.3 percent of the aliphatic carbon atoms present.

Isomerization unit 110 may produce a second hydrocarbon stream that includes olefins and paraffins. At least a portion of the second hydrocarbon stream contains branched olefins. The second hydrocarbon stream may exit isomerization unit 110 via second conduit 130 and be introduced into alkylation unit 140. Alkylation of aromatic hydrocarbons by at least a portion of branched olefins produced in isomerization unit 110 may be conducted using various types of reactors. In certain embodiments, an alkylation process may be carried out in a batch wise fashion by adding the catalyst and aromatic hydrocarbons to a reactor, heating the mixture to a reaction temperature, and then adding the olefinic and/or aromatic hydrocarbons to the heated mixture. Control of the reaction temperature may be obtained by circulating a heat transfer fluid through the jacket of the reactor, evaporative cooling of the aromatic hydrocarbons and/or the use of a condenser. In some embodiments, a fixed bed reactor operating in an up flow or down flow mode or a moving bed reactor operating with concurrent or countercurrent catalyst and/or hydrocarbon stream flows may be used. Fixed bed reactors may contain a single catalyst bed or multiple beds and may be equipped for the addition of olefins and cooling. Continuous removal of spent catalyst for regeneration and replacement by fresh and/or regenerated catalysts may be performed using a moving bed reactor. In other embodiments, a catalytic distillation reactor may also be used. Catalytic distillation reactors are described in U.S. Pat. No. 6,291,719 to Gao et al., entitled “Methods And Equipments Of Using Dual Functional Catalyst Of Packing Type,” and U.S. Pat. No. 5,866,736 to Chen, entitled “Process For Production Of Alkyl Benzene,” both of which are incorporated by reference herein.

Alkylation unit 140 may have several points of entry to accommodate the entry of additional process streams. As used herein, “stream entering into the alkylation unit” refers to the entry of process streams into the alkylation unit through one or more entry points. Examples of such process streams include, but are not limited to, additional streams from isomerization unit 110, a diluent hydrocarbon stream, gases, or other hydrocarbon streams that include olefins and paraffins derived from other processes.

In an embodiment, at least a portion of the olefins in the second hydrocarbon stream, produced by isomerization unit 110, is contacted with aromatic hydrocarbons (e.g., benzene) using a variety of alkylating conditions. At least a portion of the resulting alkyl aromatic hydrocarbons are monoalkylated aromatic hydrocarbons having a branched alkyl group. For example, passing a feed stream that includes 2-methyl-1-undecene into a benzene alkylation unit produces the branched alkyl aromatic hydrocarbon, 1-phenyl-2-methyl-undecane. Minimization of other alkylation products, such as dialkylation and higher alkylation products may be controlled by the process conditions (e.g., molar ratio, reaction temperatures, catalyst type and reactant concentrations) in alkylation unit 140.

In an embodiment, paraffins and other non-reactive components in the second hydrocarbon stream act as a diluent in the alkylation step. The presence of diluents in alkylation unit 140 may help control the temperature of the alkylation reaction. Diluents in alkylation unit 140 may also improve the selectivity of the alkylation process. Increased reaction temperatures in alkylation unit 140 may result in the formation of by-products (e.g., dialkylation and higher alkylation products).

The stoichiometry of the alkylation reaction requires only one mole of aromatic hydrocarbon compound per mole of total mono-olefins. Using 1:1 molar conditions, however, tends to produce mixtures of olefin oligomers and/or polymers, monoalkyl aromatic hydrocarbons, dialkyl-, trialkyl- and possibly highly polyalkylated aromatic hydrocarbons and/or unreacted olefins. A molar ratio as close to 1:1 may maximize utilization of the aromatic hydrocarbon and minimize recycle of unreacted aromatic hydrocarbons and/or unreacted olefins. The molar proportion of aromatic hydrocarbon to total mono-olefin may influence both conversion and selectivity of the alkylation reaction. In certain embodiments, a molar ratio of total aromatic hydrocarbon per mole of total mono-olefins may be set between about 3:1 and about 100:1 to maximize formation of monoalkyl aromatic hydrocarbon. In some embodiments, an aromatic hydrocarbon to olefin ratio may be from about 5:1 to about 50:1. In other embodiments, an aromatic hydrocarbon to olefin ratio may be from about 5:1 to about 35:1. In certain embodiments, an aromatic hydrocarbon to total olefin ratio may be from about 8:1 to about 30:1.

In certain embodiments, it may be desirable to adjust the olefin and paraffin concentration depending on the source of the olefin stream. A paraffinic stream may be added to a process stream that contains greater than about 50 percent mono-olefins upstream of an alkylation unit to produce a process stream that is less than about 50 percent mono-olefins. In some embodiments, a process stream containing about 20 percent linear olefins and 80 percent paraffins may be added to a process stream containing primarily branched olefins upstream of an alkylation unit. Alkylation of aromatic hydrocarbons with the combined stream may result in a mixed stream containing branched and linear alkyl aromatic hydrocarbons.

In an embodiment, an olefin content of a third hydrocarbon stream ranges between about 1 percent and about 99 percent relative to the total hydrocarbon content. In certain embodiments, an olefin content of a third hydrocarbon stream may be between about 5 percent and about 15 percent relative to the total hydrocarbon content. In other embodiments, an olefin content of a third hydrocarbon stream may be greater than 80 percent relative to the total hydrocarbon stream.

In an embodiment, alkylation conditions for alkylation of aromatic hydrocarbons in alkylation unit 140 may include the use of a Friedel-Crafts catalyst type. The Friedel-Crafts catalyst may be an acidic inorganic material. Examples of acidic inorganic materials include, but are not limited to, mineral acids such as sulfuric acid containing less than about 10 percent water, hydrofluoric acid containing less than about 10 percent water, liquefied anhydrous hydrogen fluoride, and/or other inorganic materials used in combination with hydrofluoric acid. Other inorganic materials may include, but are not limited to, Lewis acids such as anhydrous aluminum chloride, anhydrous aluminum bromide and boron trifluoride.

In certain embodiments, an alkylation catalyst may be a molecular sieve such as ITQ-21 in the acidic form. The synthesis and structures of molecular sieve catalysts are described by Corma, et al. in “A large-cavity zeolite with wide pore windows and potential as an oil refining catalyst,” Nature, 2002, 418:514.

In certain embodiments, an alkylation catalyst used in alkylation unit 140 is based on a zeolite catalyst that may be modified with a metal or metal compound. In other embodiments, a zeolite catalyst may not be modified with a metal or metal compound. A zeolite catalyst may have at least one channel with a crystallographic free channel diameter ranging from greater than about 4 Å to less than about 9 Å with the understanding that when the pores have an elliptical shape. The larger pore size dimension is the dimension to be considered. In an embodiment, a pore size dimensions may range between about 5.5 Å to about 7 Å. Pore size dimensions of zeolites are described by W. M. Meier et al., in “Atlas of Zeolite Structure Types,” Fourth revised edition, 1996, Elsevier, which is incorporated by reference herein.

Suitable alkylation catalysts are zeolites in acidic form. In an embodiment, an alkylation catalyst may include mordenite, ZSM-4, ZSM-5, ZSM-11, ZSM-12, ZSM-20, zeolite Y, Beta, offretite, gemelinite and cancrinite. Alkylation catalysts, in certain embodiments, may have a NES zeolite structure type that has high selectivity to a 2-aryl alkane, including isotypic framework structures such as NU-87 and gottardiite. NES structures are described in U.S. Pat. No. 5,102,641 to Casci et al. entitled “Zeolite NU-87”, and W. M. Meier et al., “Atlas of Zeolite Structure Types,” Fourth revised edition, 1996, Elsevier, both of which are incorporated by reference herein. A zeolite alkylation catalyst is described in U.S. patent application Ser. No. 10/075,318 entitled “A Process For Preparing (Branched-Alkyl) Arylsulfonates and (Branched-Alkyl) Arylsulfonate Composition,” U.S. Pat. No. 6,111,158 to Marinangeli et al. entitled “Process For Producing Arylalkanes At Alkylation Conditions Using A Zeolite Having a NES Zeolite Structure Type” and U.S. Pat. No. 5,041,402 to Schoennagel et al., entitled “Thermally Stable Noble Metal-Containing Zeolite Catalyst,” all of which are incorporated herein by reference.

=An alkylation catalyst may include a mixture of silica tetraoxide (SiO₄) and alumina tetraoxide (AlO₄) of tetrahedral structure types. The molar ratio of Si to Al in the alkylation catalyst may be defined as the molar ratio of the SiO₄ tetrahedra to the AlO₄ tetrahedra (i.e. the framework Si/Al molar ratio). In certain embodiments, a Si to Al molar ratio is at least 5:1. In some embodiments, a molar ratio of Si to Al is at most 500:1. The molar ratio of Si to Al is, in other embodiments, at most 100:1. In certain embodiments, when the zeolitic alkylation catalyst is of the NES zeolite structure type, a molar ratio of Si to Al is in the range of from 5:1 to 25:1. In other embodiments of a NES zeolite alkylation catalyst, a molar ratio of Si to Al may range from 10:1 to 20:1.

The alkylation catalyst may have at least a portion of the cationic sites occupied by ions other than alkali or alkaline earth metal ions. Examples of replacing ions include, but are not limited to, organic cations, ammonium, hydrogen, zinc, copper and rare earth elements. In certain embodiments, an alkylation catalyst may be at least partly in the hydrogen form, i.e. acidic form. In some embodiments, hydrogen ions may occupy at least 90 percent of available cationic sites. In other embodiments, hydrogen ions may occupy at least 10 percent of available cationic sites. Hydrogen ions, in certain embodiments, may occupy at least 50 percent of available cationic sites. Occupation of available cationic sites by hydrogen may be accomplished by exchange of alkali metal ions or other ions for a hydrogen ion precursor (e.g., ammonium ions), which upon calcination may yield the hydrogen form of the zeolite catalyst.

The zeolitic alkylation catalyst may be subjected to various chemical treatments, (e.g., alumina extraction) and/or be combined with one or more metal components. Metal components may include, but are not limited to, Group IIIB, Group IVB, Group VIB, Group VIIB, Group VIII, and/or Group IIB metals. The zeolites may be subjected to thermal treatment. Thermal treatments may include, but are not limited to, steaming and/or calcination in air, hydrogen or an inert gas (e.g., nitrogen or helium).

Zeolites, having a NES zeolite structure type prepared in the presence of organic cations, may not be sufficiently catalytically active for alkylation. The zeolite catalyst may be activated, for example, by heating in an inert atmosphere at above about 500° C. for one hour, ion exchanging with ammonium salts, and calcining at above about 500° C. in air. Although, the presence of organic cations in the forming solution may not be necessary for forming zeolites having an NES zeolite structure type; their presence appears to favor the formation of the NES type of zeolite structure.

Catalyst used in alkylation processes may be used alone or suitably combined with a refractory oxide or porous matrix materials that serve as a binder material. Examples of suitable binder materials include, but are not limited to, natural clays (e.g., bentonite, montmorillonite, attapulgite, and/or kaolin), alumina, silica, silica-alumina, hydrated alumina, titania, zirconia, silica-magnesia, silica-zirconia, silica-titania, silica-alumina-thoria, silica-alumina-zirconia or mixtures thereof. The alkylation catalyst may be used as a powder or a compounded form (e.g., extruded or shaped). Extruded forms may include, but are not limited to pellets, cylinders, rings or lobes. Shaped forms may include, but are not limited to, spheres, wagon wheels or polylobe structures. The amount of binder used may range from about 0 to about 90 weight percent of the catalyst.

In alkylation unit 140, at least a portion of the olefins in the second hydrocarbon stream and at least a portion of the aromatic hydrocarbons may be reacted under alkylation conditions in the presence of the alkylation catalyst. In certain embodiments, an alkylation catalyst may be used in a quantity of from about 0.5 percent to about 100 percent by weight relative to the weight of the branched olefins. In other embodiments, an alkylation catalyst may be used in a quantity of from about 1 percent to about 50 percent by weight, relative to the weight of the branched olefins applied. Reaction temperatures for the alkylation reaction may range between greater than about 30° C. and less than about 300° C. In certain embodiments, reaction temperatures may range between greater than about 100° C. to less than about 250° C. Alkylation reactions may be conducted in at least a partial liquid phase, in an all-liquid phase or at supercritical conditions. In certain embodiments, pressures in the alkylation unit are sufficient to maintain reactants in the liquid phase. The pressure used in alkylation unit 140 may depend upon the identity of olefin, the identity of the aromatic hydrocarbon and/or the temperature of the reaction. Pressures in alkylation unit 140 may range between about 3 atmospheres and about 70 atmospheres (304 kPa-7095 kPa). In certain embodiments, pressures may range between about 20 atmospheres and about 35 atmospheres (2025-3545 kPa).

Alkylation conditions in alkylation unit 140 may be maintained to minimize skeletal isomerization of the branched olefins. Alkylation conditions may also be maintained to produce alkyl aromatic hydrocarbons with an alkyl group that corresponds to the branching of the olefins produced in isomerization unit 110. “Skeletal isomerization during alkylation,” as used herein, refers to an isomerization that occurs during alkylation that changes the position of the branching in the olefin or alkyl aromatic hydrocarbon product. Accordingly, alkylation conditions may be set such that the number of quaternary aliphatic carbon atoms in the olefin and the alkyl aromatic hydrocarbon product may remain unchanged during the alkylation reaction.

A general class of branched alkyl aromatic compounds produced in alkylation unit 140 may be characterized by a chemical formula R-Y, where Y represents an aromatic hydrocarbyl radical (e.g., a phenyl radical); and R represents a radical derived from the olefin produced in isomerization unit 110. The olefin may have a carbon number in the range from 7 to 16. In certain embodiments, an olefin carbon number may range from 10 to 16. An olefin carbon number may, in other embodiments, range from 10 to 13. R may be a branched alkyl radical. Branches on the alkyl radical may include, but are not limited to, methyl, ethyl and/or longer carbon chains. The average number of branches per alkyl molecule present in the alkyl composition may be equal to the number of branches in the olefin produced in isomerization unit 110 (e.g., between 0.1 and 2.0).

The alkylation reaction mixture stream may enter separator 160 via third conduit 150. In separator 160 at least two streams, an unreacted hydrocarbons stream and an alkyl aromatic hydrocarbon stream may be produced. Separation of at least a portion of unreacted hydrocarbons from the produced branched alkyl aromatic hydrocarbons may be accomplished by methods generally known (e.g., distillation, solid/liquid separation, adsorption, solvent extraction).

In certain embodiments, at least a portion of an alkylation reaction stream produced in alkylation unit 140 may pass through a solid/liquid separator (e.g., filter or centrifuge) to remove a solid catalyst and produce a catalyst-free alkylation stream. Next, at least a portion of the catalyst-free alkylation stream may pass through one or more distillation columns to produce an alkyl aromatic hydrocarbons stream, aromatic hydrocarbons stream, paraffins and unreacted olefins stream or combinations thereof. In certain embodiments, at least a portion of the aromatic hydrocarbons stream and at least a portion of the paraffins and unreacted olefins stream may be produced as one stream and further separated into an aromatic hydrocarbons stream and a paraffins and unreacted olefins stream using distillation techniques. At least a portion of the separated aromatic hydrocarbons stream may be recycled to alkylation unit 140 via fourth conduit 170. At least a portion of the separated paraffins and unreacted olefins may enter dehydrogenation unit 180 via fifth conduit 185. In other embodiments, after separation from the alkylation reaction mixture, at least a portion of the paraffins and unreacted olefins stream may be transferred to another processing unit and/or a storage vessel.

In certain embodiments, an average carbon number of the hydrocarbons in the paraffins and unreacted olefins stream may range from 7 to 16. In some embodiments, an average carbon number of the paraffins and unreacted olefins stream may range from 10 to 16. In other embodiments, an average carbon number of the hydrocarbons in the paraffins and unreacted olefins stream may range from 10 to 13.

In some embodiments, an alkyl aromatic hydrocarbons stream may contain unwanted higher molecular weight products. Passing at least a portion of the alkyl aromatic hydrocarbons stream through a distillation column to separate the alkyl aromatic hydrocarbon product from the heavier side products may further purify the alkyl aromatic hydrocarbon stream. At least a portion of the purified alkyl aromatic hydrocarbon product stream may be transferred through sixth conduit 190 to be stored on site, sold commercially, transported off-site and/or utilized in other processing units.

Referring back to System 100 depicted in FIG. 1, at least a portion of the paraffins and unreacted olefins stream may be introduced into dehydrogenation unit 180 via fifth conduit 185. At least a portion of the unreacted paraffins in the hydrocarbon stream may be dehydrogenated to produce an olefinic hydrocarbon stream by use of a catalyst selected from a wide range of catalyst types. For example, the catalyst may be based on a metal and/or a metal compound deposited on a porous support. The metal or metal compound may included, but is not limited to, chrome oxide, iron oxide and noble metals. “Noble metals” as used herein, refer to metals of the group that includes platinum, palladium, iridium, ruthenium, osmium and rhodium.

Techniques of preparing catalysts, for performing the dehydrogenation step and for performing associated separation steps are generally known. For example, suitable procedures for preparing catalysts and performing the dehydrogenation step are described in U.S. Pat. No. 5,012,021 to Vora et al., entitled “Process For the Production of Alkyl Aromatic Hydrocarbons Using Solid Catalysts;” U.S. Pat. No. 3,274,287 to Moore et al., entitled “Hydrocarbon Conversion Process and Catalyst;” U.S. Pat. No. 3,315,007 to Abell et al., entitled “Dehydrogenation of Saturated Hydrocarbons Over Noble-Metal Catalyst;” U.S. Pat. No. 3,315,008 to Abell et al., entitled “Dehydrogenation of Saturated Hydrocarbons Over Noble-Metal Catalyst;” U.S. Pat. No. 3,745,112 to Rausch, entitled “Platinum-Tin Uniformly Dispersed Hydrocarbon Conversion Catalyst and Process;” U.S. Pat. No. 4,506,032 to Imai et al., entitled “Dehydrogenation Catalyst Composition” and U.S. Pat. No. 4,430,517 to Imai et al., entitled “Dehydrogenation Process Using a Catalytic Composition,” all of which are incorporated by reference herein.

Reaction conditions in dehydrogenation unit 180 may be varied to control unwanted side products (e.g., coke, dienes oligomers, cyclized hydrocarbons) and control double bond position in the olefin. In certain embodiments, temperatures may range from greater than about 300° C. to less than about 700° C. In other embodiments, a dehydrogenation reaction temperature may range from about 450° C. to about 550° C. During dehydrogenation, pressures in dehydrogenation unit 180 may range from greater than about 1.0 atmosphere (101 kPa) to less than about 15 atmospheres (1520 kPa). In certain embodiments, pressure in dehydrogenation unit 180 may range from about 1.0 atmosphere (101 kPa) to about 5.0 atmospheres (510 kPa). In order to prevent coke from forming, hydrogen may be fed into dehydrogenation unit 180 together with the paraffins and unreacted olefins stream. The hydrogen to paraffins molar ratio may be set between about 0.1 moles of hydrogen to about 20 moles of paraffins. In some embodiments, a hydrogen to paraffin molar ratio is about 1 to 10.

The amount of time (e.g., the residence time)-that a process stream remains in dehydrogenation unit 180 may determine, to some extent, the amount of olefins produced. Generally, the longer a process stream remains in dehydrogenation unit 180, the conversion level of paraffins to olefins increases until an olefin-paraffin thermodynamic-equilibrium is obtained. Residence time of the paraffins and unreacted olefins stream in dehydrogenation unit 180 may be such that the conversion level of paraffins to olefins may be kept below 50 mole percent. In certain embodiments, conversion level of paraffins to olefins may be kept in the range of from 5 to 30 mole percent. By keeping the conversion level low, side reactions may be prevented (e.g., diene formation and cyclization reactions).

Dehydrogenation unit 180 receives at least a portion of the paraffins and unreacted olefins stream from separation unit 160 and produces an olefinic hydrocarbon stream. The olefinic hydrocarbon stream may include paraffins. The concentration of the olefins in the olefinic hydrocarbon stream may be between 5 and 50 percent by weight. In certain embodiments, a concentration of olefins may range from 10 to 20 percent by weight. The olefins produced in dehydrogenation unit 180 may be predominately linear olefins. The average carbon number of the hydrocarbons in the olefinic stream may range from 7 to 16. The average carbon number of the hydrocarbons in the olefinic stream ranges, in certain embodiments, from about 10 to 16. In other embodiments, an average carbon number of the hydrocarbons in the olefinic stream may range from 10 to 13.

In certain embodiments, at least a portion of non-converted paraffins may be separated from the olefinic stream and, if desired, the non-converted paraffins may be recycled to dehydrogenation unit 180 to undergo further dehydrogenation. Such separation may be accomplished by extraction, distillation or adsorption techniques.

The olefinic hydrocarbon stream may be combined with first hydrocarbon stream in first conduit 120 of isomerization unit 110 via seventh conduit 195. The combined stream may enter isomerization unit 110 and at least a portion of the olefins present in the combined stream may be isomerized to branched olefins. In some embodiments, an olefinic hydrocarbon steam may exit dehydrogenation unit 180 and be directly introduced into isomerization unit 110 through one or more points of entry.

In certain embodiments, additional hydrocarbon-streams may be-used control reaction conditions and/or optimize the concentration of paraffins and unreacted olefins in isomerization unit 110, alkylation unit 140, and/or other processing units used to produce alkyl aromatic hydrocarbons. Referring to System 200, as depicted in FIG. 3, a first hydrocarbon stream may be introduced into isomerization unit 110 via first conduit 210. The first hydrocarbon stream may include olefins and paraffins. In certain embodiments, hydrocarbons in the first hydrocarbon stream may have an average carbon number from 7 to 16. In other embodiments, hydrocarbons in the first hydrocarbon stream may have an average carbon number from 10 to 13. The olefins may, in some embodiments, be alpha-olefins. The alpha-olefin content of the first hydrocarbon stream may be greater than about 70 percent of the total amount of olefins in the first hydrocarbon stream. In certain embodiments, a first hydrocarbon stream is derived from a Fischer-Tropsch process. In isomerization unit 110, at least a portion of the olefins in the first hydrocarbon stream may be isomerized to branched olefins to produce a second hydrocarbon stream. Conditions of the olefin isomerization may be controlled, as previously described for System 100, such that the number of carbon atoms in the olefin prior to and subsequent to the isomerization conditions is substantially the same.

At least a portion of a paraffinic hydrocarbon stream may be introduced into first conduit 210 via second conduit 220 upstream of isomerization unit 110 to produce a combined stream. The combined stream may enter isomerization unit 110 via first conduit 210. In other embodiments, a paraffinic hydrocarbon stream is introduced directly into isomerization unit 110 through one or more points of entry.

At least a portion of the olefins in the combined stream may be isomerized to branched olefins in isomerization unit 110 to produce a second hydrocarbon stream. Addition of the paraffinic hydrocarbon stream may be used to optimize the olefin concentration in isomerization unit 110 and to control the extent of branching in the produced olefins. Concentration of paraffins in the paraffinic hydrocarbon stream may be between about 10 percent and about 99 percent by weight. In certain embodiments, a paraffin concentration may range between about 10 percent and about 50 percent by weight. In some embodiments, a paraffin concentration may range between about 25 percent and about 75 percent by weight. In other embodiments, a paraffinic stream may include olefins. An olefin concentration in the hydrocarbon stream may be between 20 and 80 percent.

The second hydrocarbon stream may exit isomerization unit 110 and be introduced into alkylation unit 140 via third conduit 230. The second hydrocarbon stream may include branched olefins. At least a portion of a third hydrocarbon stream may be introduced into third conduit 230 via fourth conduit 240 upstream of alkylation unit 140 to form a mixed stream. The mixed stream may be then introduced into alkylation unit 140 via third conduit 230. At least a portion of the olefins in the mixed stream may alkylate at least a portion of aromatic hydrocarbons using process conditions as previously described for System 100. In some embodiments, a third hydrocarbon stream may be introduced directly into alkylation unit 140 through one or more points of entry. It should be understood that dilution of the process stream may be accomplished by adding a diluent stream through second conduit 220 only, fourth conduit 240 only, directly into alkylation 140 only or by combinations thereof.

The third hydrocarbon stream in fourth conduit 240 may be used to optimize the olefin concentration in alkylation unit 140 to maximize monoalkylation of the aromatic hydrocarbons and the amount of alkyl branching in the product. The third hydrocarbon stream may be from the same source as the first hydrocarbon stream. Alternatively, the third hydrocarbon stream may be a hydrocarbon stream that includes olefins, paraffins, and/or hydrocarbon solvents derived from another source. The third hydrocarbon stream may be derived from the same source as the paraffinic hydrocarbon stream introduced via second conduit 220.

The third hydrocarbon stream may include olefins and paraffins. In certain embodiments, an average carbon number of the hydrocarbons in the third hydrocarbon stream ranges from 7 to 16. The paraffin content of the third hydrocarbon stream may be between about 45 percent and about 99 percent by weight. In certain embodiments, a paraffin content of the third hydrocarbon stream may be between about 60 percent and about 90 percent by weight. In other embodiments, a paraffin content may be greater than about 80 percent by weight.

In an embodiment, an olefin content of a third hydrocarbon stream ranges between about 1 percent and about 99 percent relative to the total hydrocarbon content. In certain embodiments, an olefin content of a third hydrocarbon stream may be between about 5 percent and about 15 percent relative to the total hydrocarbon content. In other embodiments, an olefin concentration of the third hydrocarbon stream may be greater than about 80 percent by weight.

In some embodiments, the third hydrocarbon stream may include linear olefins. Addition of a stream that includes linear olefins downstream from the isomerization unit allows the creation of an alkylation feed stream that includes a mixture of linear and branched olefins. By introducing a stream including branched and linear olefins into alkylation unit 140, a mixture of branched and linear alkyl aromatic products may be obtained. Varying the amount of linear olefins added to the alkylation feed stream may control the ratio of linear to branched alkyl aromatic products. A mixture of branched and linear alkyl aromatic hydrocarbons may have improved properties when converted to alkyl aromatic surfactants. Examples of improved properties include, but are not limited to, low skin and eye irritation, foaming properties, biodegradability, cold-water solubility and cold-water detergency. Applications for these surfactants include, but are not limited to, personal care products, household and industrial laundry products, hand dishwashing products, machine lubricant additives and lubricating oil formulations.

The alkylation reaction mixture stream may enter separator 160 via fifth conduit 250. Separation of at least a portion of paraffins and at least a portion of olefins from the alkylation reaction mixture may be accomplished as previously described for System 100. In separator 160, at least two streams may be produced, an unreacted hydrocarbons stream and an alkyl aromatic hydrocarbon product stream. At least a portion of the unreacted hydrocarbons stream may be separated into an aromatic hydrocarbons stream and a paraffins and unreacted olefins stream using generally known techniques (e.g., distillation, solid/liquid separation, absorption, solvent extraction). At least a portion of the separated aromatic hydrocarbons may be transferred to alkylation unit 140 via sixth conduit 260. At least a portion of the alkyl aromatic hydrocarbons product stream may be transferred through seventh conduit 270 to be stored on site, sold commercially, transported off-site, and/or utilized in other processing units (e.g., sulfonation unit).

At least a portion of the separated paraffins and unreacted olefins may be transferred to dehydrogenation unit 180 via eighth conduit 280. Hydrocarbons in the unreacted olefin and paraffins stream may have the same average number of carbon atoms as the hydrocarbons in one or more hydrocarbon streams that may enter the isomerization unit and/or alkylation unit. The average carbon number of the hydrocarbons in the unreacted olefin and paraffins stream may range from 7 to 16. In certain embodiments, an average carbon number of the unreacted olefin and paraffins may range from 10 to 16. An average carbon number of the hydrocarbons in the unreacted olefin and paraffins may range stream range, in some embodiments, from 10 to 13.

As depicted in FIG. 3, at least a portion of the paraffins and unreacted olefins stream may enter dehydrogenation unit 180 via eighth conduit 280. At least a portion of the paraffins in the hydrocarbon stream may be dehydrogenated using process conditions as previously described for System 100. At least a portion of the resulting olefinic hydrocarbon stream may exit dehydrogenation unit 180 and be transported to another processing unit and/or a storage vessel via ninth conduit 290.

In certain embodiments, at least a portion of non-converted paraffins may be separated from dehydrogenated compounds in the olefinic stream. At least a portion of the non-converted paraffins may be recycled to dehydrogenation unit 180 to undergo further dehydrogenation. Such separation may be accomplished by extraction, distillation or, adsorption techniques.

In System 200 as depicted in FIG. 3, at least a portion of the olefinic hydrocarbon stream may exit dehydrogenation unit 180 via ninth conduit 290 and be combined with the first hydrocarbon stream in first conduit 210 upstream of isomerization unit 110 to produce a combined stream. The combined stream may be introduced into-isomerization unit 110 via first conduit 210 and at least a portion of the olefins in the combined stream may be isomerized to branched olefins. In some embodiments, an olefinic hydrocarbon stream may be-introduced directly into isomerization unit 110 via one or more points of entry. Alternatively, at least a portion of the olefinic hydrocarbon stream may be combined with a second hydrocarbon stream in third conduit 230 downstream of isomerization unit 110 to produce a mixed stream. Depending on the dehydrogenation conditions, the mixed stream may include linear olefins. Addition of the olefinic hydrocarbon stream with the second hydrocarbon stream may produce a mixed stream that includes both linear and branched olefins.

In certain embodiments, a unit that combines the processes of dehydrogenation and isomerization in the production of branched olefins from a predominately paraffinic feed stream may be desired. By combining two processes into one unit, a more economically valuable process may result (e.g., greater utilization of a mostly paraffinic hydrocarbon stream). Processes using one unit to produce branched olefins with a substantially minimal amount quaternary and/or tertiary carbon atoms are described. Branched olefins may be converted to alkyl aromatic hydrocarbons that may be used to manufacture other commercially valuable products (e.g., surfactants).

In certain embodiments, a first hydrocarbon stream that includes paraffins and olefins may be introduced into a dehydrogenation-isomerization unit. The dehydrogenation-isomerization unit may replace two independent units (e.g., an isomerization unit and a dehydrogenation unit). The dehydrogenation-isomerization unit may dehydrogenate paraffins to olefins, and isomerize the resulting olefins and/or initial olefins present in the hydrocarbon stream to branched olefins. In an embodiment, a catalyst may perform the dehydrogenation-isomerization of the hydrocarbons in the first hydrocarbon stream. In certain embodiments, dehydrogenation-isomerization catalyst may be a single catalyst. The catalyst, in some embodiments, may be a mixture of two catalysts (e.g., a dehydrogenation catalyst and an isomerization catalyst). In other embodiments, two separate catalysts located in different zones or in a stacked bed configuration in one dehydrogenation-isomerization unit may perform the dehydrogenation-isomerization process. As used herein, “a dehydrogenation-isomerization catalyst” may be one or more catalysts.

In certain embodiments, a dehydrogenation-isomerization unit may have several points of entry to accommodate different process streams. Process streams may be from other processing units and/or storage units. Examples of process streams include, but are not limited to, a diluent hydrocarbon stream, and/or other hydrocarbon streams that include olefins and paraffins derived from other processes. As used herein, “entry into the dehydrogenation-isomerization unit” refers to entry of process streams into the dehydrogenation-isomerization unit through one or more entry points.

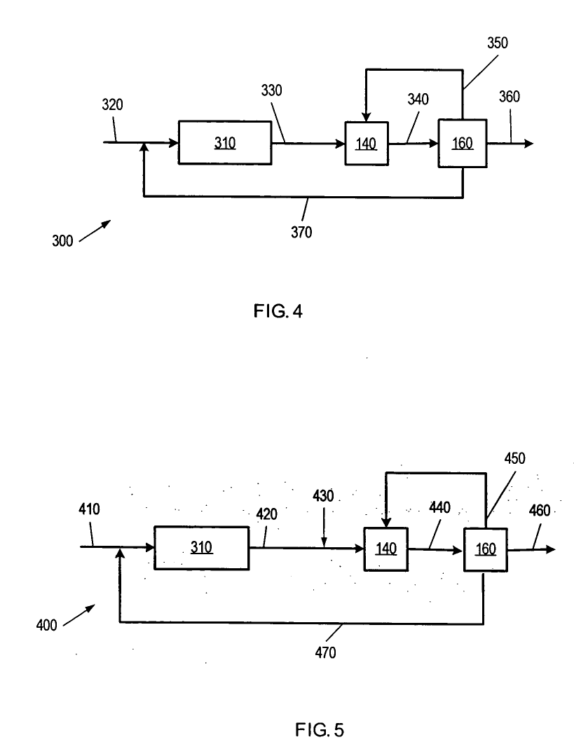

Referring to System 300, as depicted in FIG. 4, a first hydrocarbon stream, including a mixture of olefins and paraffins, may be introduced into dehydrogenation-isomerization unit 310 via first conduit 320. In certain embodiments, hydrocarbons in the first hydrocarbon stream may have an average carbon number from 7 to 16. In other embodiments, hydrocarbons in the first hydrocarbon stream may have an average carbon number from 10 to 13. In some embodiments, a first hydrocarbon stream includes alpha-olefins. An alpha-olefins content of the first hydrocarbon stream may be greater than about 70 percent of the total amount of olefins in the first hydrocarbon stream. In certain embodiments, a first hydrocarbon stream may be produced from a Fischer-Tropsch process.

In dehydrogenation-isomerization unit 310, at least a portion of the paraffins in the first hydrocarbon stream may be dehydrogenated to olefins. At least a portion of the resulting olefins and at least a portion of the olefins that were already present in the feed stream may be isomerized to produce a second hydrocarbon stream. The isomerization process converts linear olefins (e.g., unbranched olefins) into branched olefins.

The catalyst used for the dehydrogenation-isomerization of the first hydrocarbon stream may be based on a zeolite catalyst modified with one or more metals or metal compounds. The catalyst used in dehydrogenation-isomerization unit 310 to treat the olefins in the first hydrocarbon stream may be effective for skeletally isomerizing linear olefins in the process stream into olefins having an average number of branches per olefin molecule chain of between about 0.1 and about 2.5.

The dehydrogenation-isomerization catalyst may contain a zeolite having at least one channel with a crystallographic free channel diameter ranging from greater than about 4.2 Å and less than about 7 Å, measured at room temperature, with essentially no channel present which has a free channel diameter which is greater than about 7 Å. The catalyst may contain at least one channel having a crystallographic free diameter at the entrance of the channel within the stated range. The catalyst may not have a diameter at the entrance of a channel, which exceeds the 7 Å upper limit of the range. Zeolites possessing channel diameters greater than about 7 Å may be susceptible to undesirable aromatization, oligomerization, alkylation, coking and/or by-product formation of the olefins in the hydrocarbon stream. In some embodiments, a zeolite may not contain a channel having a free diameter along either of the x or y planes of greater than about 4.2 Å. The small channel size may prevent diffusion of the olefin into and out of the channel pore once the olefin becomes branched. Thus, the zeolite must have at least one channel with free diameters of that channel within the range of greater than about 4.2 Å and less than about 7 Å.

In an embodiment, an olefin molecule, due to its high carbon chain length, may not have to enter into the zeolite channel, diffuse through, and exit the other end of the channel. The rate of branching seen when passing the olefin across the zeolite may not correspond to the theoretical rate of branching if each olefin molecule were to pass through the channels. Most of the olefins may partially penetrate the channel for a distance effective to branch the portion of the chain within the channel and subsequently withdraw from the channel once isomerized. In an embodiment of a method to produce alkyl aromatic hydrocarbons, olefin molecules in a hydrocarbon stream may predominately have a structure which is branched at the ends of the olefin carbon backbone, and substantially linear towards the center of the molecule, e.g., at least 25 percent of the carbons at the center are unbranched.

The dehydrogenation-isomerization catalyst may contain a zeolite having at least one channel with a crystallographic free channel diameter ranging from greater than about 4.2 Å and less than about 7 Å, measured at room temperature. In some embodiments, the zeolite catalyst may not have channels that have a free channel diameter greater than about 7 Å. The catalyst may contain at least one channel having a crystallographic free diameter at the entrance of the channel within the stated range. The catalyst may not have a diameter at the entrance of a channel that exceeds the 7 Å upper limit of the range. Zeolites possessing channel diameters greater than about 7 Å may be susceptible to undesirable aromatization, oligomerization, alkylation, coking and/or by-product formation of the olefins in the hydrocarbon stream. In some embodiments, a zeolite may not contain a channel having a free diameter along either of the x or y planes of greater than about 4.2 Å. A small channel size may prevent diffusion of the olefin into and out of the channel pore once an olefin becomes branched.

There may be no limit on the number of dimensions, one, two, or three, that the channel system may have. In an embodiment, a circumstance may exist where olefins may meet at the intersection of the interconnecting channels and dimerize or oligomerize. The dimerization or oligomerization may depend on several factors including, but are not limited to, size of the olefin, proximity of the interconnecting intersection to the channel entrances, size of the interconnecting intersection, temperature and flow rates. While it is unlikely that the dimer may diffuse back out of the zeolite, the dimer may coke the catalyst or crack within the channel structure, which may form by-product olefins having quaternary aliphatic carbon branching. Thus, the interconnecting channel system in a two or three-dimensional zeolite may have free diameters effective to prevent the formation of dimers, trimers or oligomers under the given processing conditions. Dimer, trimers or oligomers may produce quaternary branched by-products when cracked. In certain embodiments, all channels interconnecting to the channel within the stated range may have free diameters in both of the x and y planes of less about 4.2 Å. A free diameter of less than about 4.2 Å may eliminate the possibility of two olefin molecules contacting each other within the zeolite and dimerizing or trimerizing. In some embodiments, dimerization and/or trimerization of an olefin may not occur in zeolite catalyst that contain more an one channel, (e.g., one, two, or three dimensional or even intersecting on different planes) that do not interconnect.

Examples of zeolites, with a channel-size between about 4.2 Å and 7.0 Å, include, but are not limited to, molecular sieves, ferrierite, A1PO-31, SAPO-11-, SAPO-31, SAPO-41, FU-9, NU-10, NU-23, ZSM-12, ZSM-22, ZSM-23, ZSM-35, ZSM-48, ZSM-50, ZSM-57, SUZ-4A, MeAPO-11, MeAPO-31, MeAPO-41, MeAPSO-11, MeAPSO-31, and MeAPSO-41, MeAPSO-46, ELAPO-11, ELAPO-31, ELAPO-41, ELAPSO-11, ELAPSO-31, and ELAPSO-41, laumontite, cancrinite, offretite, hydrogen form of stilbite, the magnesium or calcium form of mordenite and partheite. Isotypic structures of the zeolite frameworks, known under other names, may be considered equivalent. An overview describing the framework compositions of many of these zeolites is provided by Flanigen et al.; in “Aluminophosphate Molecular Sieves and the Periodic Table,” New Developments in Zeolite Science Technology, 1986, Kodansha Ltd., Tokyo, Japan, which is incorporated by reference herein.

Many natural zeolites such as ferrierite, heulandite and stilbite may feature a one-dimensional pore structure with a pore size at or slightly smaller than about 4.2 Å in diameter. U.S. Pat. No. 4,795,623 to Evans, entitled “Time Effective Method For Preparing Ferrierite” and U.S. Pat. No. 4,942,027 to Evans, entitled “Method for Preparing Ferrierite,” both of which are incorporated herein by reference, describe converting channels in natural zeolites to larger channels. Channels in natural zeolites may be converted to zeolites with desired larger channel sizes by removing an associated alkali metal or alkaline earth metal by methods generally known. An example of such a method includes ammonium ion exchange, optionally followed by calcination, to yield the zeolite in substantially a hydrogen form. Replacing the associated alkali or alkaline earth metal with the hydrogen form may correspondingly enlarge the channel diameter. It is understood that the channel diameter or “size” refers to the effective channel diameter or size for diffusion. Alternatively, natural zeolites with too large a channel size, such as some forms of mordenite, may be altered by substituting the alkali metal with larger ions, such as larger alkaline earth metals to reduce the channel size.

In certain embodiments, zeolites may have a ferrierite isotypic framework structure (or homeotypic). The prominent structural features of ferrierite found by x-ray crystallography may be parallel channels in the alumino-silicate framework, which are roughly elliptical in cross-section. Examples of such zeolites having the ferrierite isotypic framework structure are described in European Patent No. 55 529 to Seddon et al., entitled “Zeolites;” U.S. Pat. No. 4,578,259 to Morimoto et al., entitled “Process For Preparing A Crystalline Aluminosilicate;” European Patent No. 103 981 to Whittam, entitled “Zeolites;” U.S. Pat. No. 4,016,245 to Plank et al., entitled “Crystalline Zeolite And Method Of Preparing Same” and U.S. Pat. No. 4,375,573 to Young et al., entitled “Selective Production And Reaction of P-Disubstituted Aromatics Over Zeolite ZSM-48,” all of which are incorporated by reference herein.

In an embodiment, a hydrogen form of ferrierite (H-ferrierite) may be considered to be substantially one-dimensional, having parallel running channels with elliptical channels having free diameters of 4.2 Å times 5.4 Å along the x and y planes in the [001] view. The channels may be large enough to permit entry of a linear olefin and diffusion out of or through the channel of the methyl branched isoolefin and small enough to retard coke formation. Preparation of H-ferrierite catalysts are described in U.S. Pat. No. 5,985,238 to Pasquale et al., entitled “Process For Preparing Ferrierite;” U.S. Pat. No. 4,251,499 to Nanne et al.; entitled “Process For The Preparation Of Ferrierite;” U.S. Pat. No. 4,795,623 to Evans, entitled “Time Effective Method For Preparing Ferrierite” and U.S. Pat. No. 4,942,027 to Evans, entitled “Method for Preparing Ferrierite,” all of which are incorporated by reference herein.

In certain embodiments, a dehydrogenation-isomerization catalyst may be combined with a refractory oxide that serves as a binder material. Suitable refractory oxides include, but are not limited to, natural clays (e.g., bentonite, montmorillonite, attapulgite, and kaolin), alumina, silica, silica-alumina, hydrated alumina, titania, zirconia or mixtures thereof.

Examples of alumina binders may include, but are not limited to pseudoboehmite, gamma and bayerite aluminas. The alumina binders may be commercially available (e.g., LaRoche Chemicals manufactures. VERSAL® aluminas and Sasol manufactures CATAPAL® aluminas). In an embodiment, high-dispersity alumina powders may be used as alumina binders when extrusion is utilized for catalyst preparation. High-dispersity alumina powders may have a dispersity of greater than 50 percent in an aqueous acid dispersion having an acid content of 0.4-milligram equivalents of acid (acetic) per gram of powder. Such high-dispersity aluminas may be exemplified by CATAPAL® D alumina manufactured by Sasol.

The weight ratio of zeolite to binder material may range from about 10:90 to about 99.5:0.5. In some embodiments, a weight ratio may range from about 75:25 to about 99:1. In other embodiments, a weight ratio of zeolite to binder material may range from about 80:20 to about 98:2. In certain embodiments, a weight ratio of zeolite to binder material may range from about 85:15 to about 95:5 on an anhydrous basis.

In certain embodiments, a dehydrogenation-isomerization catalyst may be prepared with one or more monocarboxylic acids and/or inorganic acids. In addition to the monocarboxylic and/or inorganic acids, at least one organic acid with at least two carboxylic acid groups (“polycarboxylic acid”) may be used. Monocarboxylic acids may have a substituted or unsubstituted hydrocarbyl group having 1 to 20 carbon atoms. The hydrocarbyl group may be aliphatic, cyclic or aromatic. Examples of monocarboxylic acids having 1 to 20 carbon atoms include, but are not limited to, acetic acid, formic acid, propionic acid, butyric acid, caproic acid, glycolic acid, lactic acid, hydroxylbutyric acid, hydroxycyclopentanoic acid, salicylic acid, mandelic acid, benzoic acid and fatty acids. Examples of inorganic acids include, but are not limited to, nitric acid, phosphoric acid, sulfuric acid and hydrochloric acid.

A polycarboxylic acid may, in certain embodiments, be an organic acid with two or more carboxylic acid groups attached through a carbon-carbon bond linkage to a hydrocarbon segment. The linkage may be at any portion of the hydrocarbon segment. The polycarboxylic acid may have a hydrocarbon segment ranging from 0 to 10 carbon atoms. The hydrocarbon segment may be aliphatic, cyclic or aromatic. The hydrocarbon segment may have zero carbon atoms for oxalic acid with two carboxylic acid groups attached through the carbon-carbon bond. Examples of the polycarboxylic acids include, but are not limited to, tartaric acid, citric acid, malic acid, oxalic acid, adipic acid, malonic acid, galactaric acid, 1,2-cyclopentane dicarboxylic acid, maleic acid, fumaric acid, itaconic acid, phthalic acid, terephthalic acid, phenylmalonic acid, hydroxyphthalic acid, dihydroxyfumaric acid, tricarballylic acid, benzene-1,3,5-tricarboxylic acid, isocitric acid, mucic acid and glucaric acid. The polycarboxylic acids may be any isomers of the above acids or any stereoisomers of the above acids. In an embodiment, polycarboxylic acids with at least two carboxylic acid groups and at least one hydroxyl group are used. In an embodiment, a polycarboxylic acids used are citric acid, tartaric acid and malic acid.

The metals incorporated into the dehydrogenation-isomerization catalysts may be metals that promote the oxidation of coke in the presence of oxygen at a temperature greater than about 250° C. and the dehydrogenation of paraffins. “Metal(s),” as used herein, refer to metals of a zero oxidation state and/or higher oxidation states (e.g., metal oxides).

The metals used in the dehydrogenation-isomerization catalyst may be transition and rare earth metals. Coke oxidation-promoting metals include, but are not limited to, Groups IB, VB, VIIB, VIIB, VIII of the transition metal series of the Periodic Table and/or combinations thereof. In certain embodiments, Pd, Pt, Ni, Co, Mn, Ag, Cr and/or combinations thereof may be used in the dehydrogenation-isomerization catalyst.

The amount of the metal introduced may range from about 5 parts per million (“ppm”) up to about 15 percent by weight. In certain embodiments, an amount of metal may range from about 5 ppm to about 10 percent by weight. In some embodiments, an amount of metal may range from about 5 ppm to about 5 percent by weight.

Smaller amounts of metals may be incorporated into the zeolite and/or binder, when noble metals such as platinum and/or palladium are used. In certain embodiments, an amount of noble metals may range from about 5 ppm to about 2 percent by weight, basis metal, of the final catalyst. In some embodiments, an amount of noble metals may range from about 5 ppm to about 1 percent by weight, basis metal, of the final catalyst. In other embodiments, an amount of noble metal(s) may range from about 5 ppm to about 3000 ppm of a final catalyst. An amount of noble metal(s) used in a dehydrogenation-isomerization catalyst may, in certain embodiments, range from about 5 ppm to about 2000 ppm of a final catalyst. The amount of noble metal(s) sufficient to promote regeneration without deteriorating the performance of the catalyst may be in the range from about 30 ppm to about 100 ppm. Higher amounts of platinum and/or palladium (e.g., greater than about 2% by weight) may have an adverse effect on the run life, olefin isomerization activity and/or selectivity of the catalyst.

In an embodiment, zeolite powder and alumina powder may be mixed (e.g., mulling) with water and one or more compounds of the catalytic metal; and the resulting mixture may be formed into a pellet. Catalysts prepared by mulling may have superior olefin isomerization performance over catalysts prepared by impregnation. The term “mulling,” as used herein, refers to mixing of powders to which sufficient water has been added to form a thick paste and wherein the mixing is accompanied by concomitant shearing of the paste. Commercially available mullers such as the Lancaster Mix Muller and the Simpson Mix Muller may be used.

The pellet may be formed, in some embodiments, by extrusion. One or more peptizing acid, (e.g., nitric acid, acetic acid, citric acid or mixtures thereof) may be added to the mixture and optional extrusion aids such as cellulose derivatives (e.g., METHOCEL®F4M, hydroxypropyl methylcellulose, manufactured by The Dow Chemical Company) may be utilized. The amounts of peptizing acid used may be determined by routine experimentation to provide a plastic, extrudable material. “Pellets,” as used herein, refers to any shape or form as long as the extruded materials are consolidated.

In certain embodiments, a noble metal such as platinum and/or palladium may be added to the zeolitic catalyst after the catalyst is pelletized. Common metal incorporation methods known to those skilled in the art (e.g., impregnation, noble metal ion exchange, co-mulling) may be used to produce a working catalyst useful in dehydrogenation-isomerization of paraffins. Addition of noble metals to the catalyst may aid in the dehydrogenation reaction of paraffins. Pellets containing noble metals may be calcined at a temperature ranging from about 200° C. to about 700° C. In certain embodiments, calcination temperatures may range from about 300° C. to about 600° C. In some embodiments, calcination temperatures may range from about 450° C. to about to about 525° C.

The dehydrogenation-isomerization catalyst may be contacted with the first hydrocarbon stream in dehydrogenation isomerization unit 310 under a variety of conditions to dehydrogenate the paraffins and isomerize the resulting olefins. In dehydrogenation-isomerization unit 310, reaction temperatures may range from about 300° C. to about 700° C. The reaction temperature, in some embodiments, may range from about 350° C. to about 550° C. A total pressure of dehydrogenation-isomerization unit 310 during the reaction may range from than about 0.10 atmosphere (10 kPa) to about 15.0 atmospheres (1520 kPa). In order to prevent coking, hydrogen may be fed together with the first hydrocarbon stream. Hydrogen gas and paraffins present in the first hydrocarbon stream may be fed at a hydrogen gas to paraffin molar ratio in the range of from about 0.1 to about 20. In certain embodiment, the hydrogen gas to paraffin molar ratio may be in the range of about 1 to about 10.

Residence time in dehydrogenation-isomerization unit 310 may be such that conversion level of the paraffins to olefins may be kept below 40 mole percent. In an embodiment, a conversion level may range from 5 mole percent to 30 mole percent. By keeping the conversion level low, side reactions may be minimized (e.g., diene formation and cyclization reactions). Conversion may be increased, however, by increasing the reaction temperature and/or the residence time as long as side reactions remain below acceptable limits. Olefins produced in dehydrogenation-isomerization unit 310 may have a higher degree of branching than the paraffin feed to the dehydrogenation-isomerization unit. It should be understood that the concentration of olefins produced via dehydrogenation-isomerization 310 may be limited by the thermodynamic equilibrium of olefins and paraffins at the reaction temperature.

The second hydrocarbon stream may exit dehydrogenation-isomerization unit 310 and be transferred to other processing units (e.g., alkylation) via second conduit 330. At least a portion of the second hydrocarbon stream may exit dehydrogenation-isomerization unit 310 and be introduced into alkylation unit 140 via second conduit 330 as depicted in System 300 of FIG; 4. At least a portion of the olefins in the second hydrocarbon stream may alkylate aromatic hydrocarbons using conditions previously described for System 100. Separation of at least a portion of paraffins, at least a portion of olefins and at least a portion of the aromatic hydrocarbons from the alkyl aromatic hydrocarbons may be performed as previously described for System 100.

The alkylation reaction mixture stream may enter separator 160 via third conduit 340. In separator 160 at least two streams, an unreacted hydrocarbons stream and an alkyl aromatic hydrocarbon stream may be produced. The unreacted hydrocarbons stream may be separated into an aromatic hydrocarbons stream and a paraffins and unreacted olefins stream using generally known techniques (e.g., distillation, solid/liquid separation, absorption, solvent extraction). Separated aromatic hydrocarbons may be transferred to alkylation unit 140 via fourth conduit 350. At least a portion of the unreacted olefin and paraffins stream may be combined with other process streams, sent to other processing units and/or stored on site. The alkyl aromatic hydrocarbons product stream may be transported via fifth conduit 360 to be stored on site, sold commercially, transported off-site and/or utilized in other processing units.

At least a portion of the paraffins and unreacted olefins stream may be combined via sixth conduit 370 with the first hydrocarbon stream in first conduit 320 to produce a combined stream. The combined stream may be introduced into dehydrogenation-isomerization unit 310 via first conduit 320 and at least a portion of the olefins in the combined stream may be isomerized to branched olefins. In some embodiments, at least a portion of the paraffins and unreacted olefins stream is introduced directly into dehydrogenation-isomerization unit 310 via one or more points of entry. Because the paraffins and unreacted olefins may be recycled to dehydrogenation-isomerization unit 310 as one stream, the process may be more efficient, resulting in an overall higher throughput. Higher throughput will increase the overall yield of the alkyl aromatic hydrocarbon.

In certain embodiments, additional hydrocarbon streams may be used to control reaction conditions and/or optimize the concentration of paraffins and unreacted olefins in dehydrogenation-isomerization unit 310 and/or alkylation unit 140.

Referring to System 400 depicted FIG. 5, a first hydrocarbon stream containing paraffins and unreacted olefins may be introduced into dehydrogenation-isomerization unit 310 via first conduit 410. In certain embodiments, hydrocarbons in the first hydrocarbon stream may have an average carbon number from 7 to 16. In other embodiments, hydrocarbons in the first hydrocarbon stream may have an average carbon number from 10 to 13. In other embodiments, first hydrocarbon stream may include alpha-olefins. The alpha-olefin content of the first hydrocarbon stream may be greater than about 70 percent of the total amount of olefins in the first hydrocarbon stream. In certain embodiments, a first hydrocarbon stream is derived from a Fischer-Tropsch process. In the dehydrogenation-isomerization unit 310 at least a portion of the paraffins in the feed stream may be dehydrogenated to form olefins. The dehydrogenation-isomerization unit may also isomerize at least a portion of the resulting olefins and at least a portion of the olefins that were already present in the feed stream. The isomerization process converts linear olefins (e.g., unbranched olefins) into branched olefins. Conditions of the isomerization may be controlled such that the number of carbon atoms in the olefin prior to and subsequent to the olefin isomerization conditions may be substantially the same.

At least a portion of the second hydrocarbon stream may exit dehydrogenation-isomerization unit 310 and enter alkylation unit 140 via second conduit 420. At least a portion of a third hydrocarbon stream may be introduced into second conduit 420 via third conduit 430 to produce a combined stream. The combined stream may be introduced into alkylation unit 140 via second conduit 420. At least a portion of the olefins in the combined stream may alkylate the aromatic hydrocarbon under the same process conditions as described for System 100, depicted in FIG. 1. In an embodiment, a third hydrocarbon stream is introduced directly into alkylation unit 140 through one or more points of entry.

The third hydrocarbon stream may be used to optimize the olefin concentration in alkylation unit 140 at a concentration sufficient to maximize monoalkylation of the aromatic hydrocarbon. In addition, the third hydrocarbon stream may optimize the linear to branched ratio of the produced alkyl aromatic hydrocarbons, as described above. The third hydrocarbon stream may be from the same source as the first hydrocarbon stream. Alternatively, the third hydrocarbon stream may be a hydrocarbon stream that includes olefins, paraffins, and/or hydrocarbon solvents derived from another source.

In certain embodiments, a third hydrocarbon stream paraffin content ranges between about 50 percent and about 99 percent relative to the total hydrocarbon content. In some embodiments, a paraffin content of the third hydrocarbon stream may range between 60 and 90 percent relative to the total hydrocarbon content. In other embodiments, a paraffin content may be greater than about 80 percent relative to the total hydrocarbon content.

In an embodiment, an olefin content of a third hydrocarbon stream ranges between about 1 percent and about 99 percent relative to the total hydrocarbon content. In certain embodiments, an olefin content of a third hydrocarbon stream may be between about 5 percent and about 15 percent relative to the total hydrocarbon content. In other embodiments, an olefin content of a third hydrocarbon stream may be greater than 80 percent relative to the total hydrocarbon content.

In an embodiment, the third hydrocarbon stream may include linear olefins. Addition of a stream that includes linear olefins downstream from the dehydrogenation-isomerization unit allows the creation of an alkylation feed stream that includes a mixture of linear and branched olefins. By introducing a stream including branched and linear olefins into alkylation unit 140, a mixture of branched and linear alkyl aromatic products may be obtained. Varying the amount of linear olefins added to the alkylation feed stream may control the ratio of linear to branched alkyl aromatic products. A mixture of branched and linear alkyl aromatic hydrocarbons may have distinct properties when converted to alkyl aromatic sulfonate surfactants. Examples of improved properties include, but are not limited to, low skin and eye irritation, foaming properties, biodegradability, cold-water solubility and cold-water detergency. Applications for the surfactants include, but are not limited to, personal care products, household and industrial laundry, hand dishwashing, machine lubricants additives, and lubricating oil formulation.

The alkylation reaction mixture stream may enter separator 160 via fourth conduit 440. Separation of at least a portion of unreacted olefins and at least a portion of paraffins from the alkyl aromatic hydrocarbons may be performed as previously described for System 100. In separator 160 at least two streams, an unreacted hydrocarbons stream and an alkyl aromatic hydrocarbons stream may be produced. The unreacted hydrocarbons stream may be separated into an aromatic hydrocarbons stream and a paraffins and unreacted olefins stream using generally known techniques (e.g., distillation, solid/liquid separation, absorption, solvent extraction). Separated aromatic hydrocarbons may be transferred to alkylation unit 140 via fifth conduit 450. At least a portion of the paraffins and unreacted olefins stream may be combined with other process streams, stored on site and/or sent to other processing units. The alkyl aromatic hydrocarbons product stream may be transported via sixth conduit 460 to be stored on site, sold commercially, transported off-site and/or utilized in other processing units.

As depicted in FIG. 5, at least a portion of the paraffins and unreacted olefins stream, via seventh conduit 470, may be introduced into first conduit 410 to produce a combined stream. The combined stream may be introduced into dehydrogenation-isomerization unit 310 via first conduit 410 and at least a portion of the paraffins in the combined stream may be dehydrogenated and isomerized to branched olefins. In an embodiment, at least a portion of an paraffins and unreacted olefins stream may be introduced directly into dehydrogenation-isomerization unit 310 via one or more points of entry. In certain embodiments, one or more combined process streams and at least a portion of an paraffins and unreacted olefins stream may be introduced directly into dehydrogenation-isomerization unit 310 through one or more points of entry.

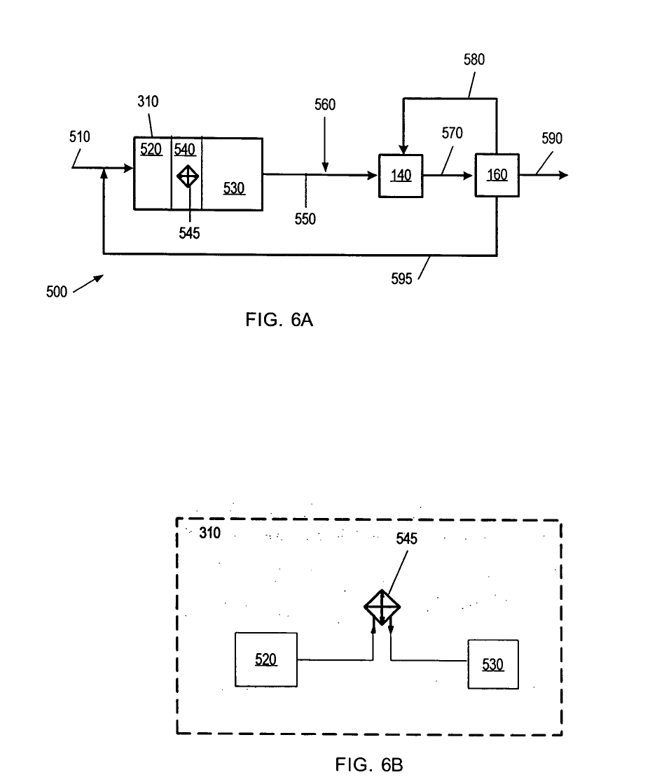

In certain embodiments, dehydrogenation-isomerization unit 310 may be separated into a plurality of zones to control reaction temperatures and/or prevent unwanted side reactions (e.g., diene formation and/or cyclization reactions). Referring to System 500, depicted in FIG. 6A, a first hydrocarbon stream containing paraffins and unreacted olefins may be introduced into dehydrogenation-isomerization unit 310 via first conduit 510. In some embodiments, a first hydrocarbon stream includes alpha-olefins. In certain embodiments, hydrocarbons in the first hydrocarbon stream may have an average carbon number from 7 to 16. In other embodiments, hydrocarbons in the first hydrocarbon stream may have an average carbon number from 10 to 13. The alpha-olefin content of the first hydrocarbon stream may be greater than about 70 percent of the total amount of olefins in the first hydrocarbon stream. In certain embodiments, a first hydrocarbon stream is produced from a Fischer-Tropsch process. Dehydrogenation-isomerization unit 310 may be divided into a plurality of zones. The plurality of zones may include, but is not limited to, a first reaction zone, a transition zone and a second reaction zone. In first reaction zone 520, at least a portion of the paraffins in the first hydrocarbon stream may be dehydrogenated to olefins to produce an olefinic stream. The process stream may then pass into second reaction zone 530. In second reaction zone 530 at least a portion of the olefins in the process stream may be isomerized to branched olefins to produce a second hydrocarbon stream.

In first reaction zone 520 the dehydrogenation catalyst may be selected from a variety of catalyst types as described previously for dehydrogenation unit 180. Reaction temperatures may range from about 300° C. to about 600° C. In some embodiments, a reaction temperature may range from about 450° C. to about 550° C. Total pressure in the dehydrogenation zone may range from about 0.10 atmosphere (10 kPa) to about 15.0 atmospheres (1520 kPa). In order to prevent coking, hydrogen may be fed together with the unreacted first hydrocarbon stream. Hydrogen and paraffins present in the unreacted first hydrocarbon steam may be fed at a hydrogen to paraffin molar ratio in the range of from about 0.1 to about 20. In an embodiment, a hydrogen to paraffin molar ratio may be in the range of about 1 to about 10.

Residence time in first reaction zone 520 may be selected such that conversion level of the paraffins to olefins may is below about 50 mole percent. In certain embodiments, conversion level of the paraffins to olefins may be kept in the range of from about 10 mole percent to about 20 mole percent. By keeping the conversion level low, side reactions may be prevented (e.g., diene formation and cyclization reactions). The olefinic hydrocarbon stream may exit first reaction zone 520, pass through transition zone 540 and enter second reaction zone 530. transition zone 540 may include heat exchanger 545. Heat exchanger 545 may reduce the temperature of the olefinic hydrocarbon stream. In an embodiment, first reaction zone 520 and second reaction zone 530 in dehydrogenation-isomerization unit 310 maybe separate units, as depicted in FIG. 6B, with heat exchanger 545 positioned between the two units.

After the olefinic hydrocarbon stream enters second reaction zone 530 at least a portion of the olefins are isomerized to branched olefins to produce a second hydrocarbon stream. Composition and level of branching of the second hydrocarbon stream may be performed by ¹H NMR analysis. In an embodiment, the olefinic stream may exit first reaction zone 520 and directly enter second reaction zone 530 where at least a portion of the olefins in the olefinic stream is isomerized to branched olefins.

A catalyst used for isomerization of the olefins to branched olefins may be the same as that described for System 100 depicted in FIG. 1. Temperatures in second reaction zone 530 may range from about 200° C. to about 500° C. In some embodiments, reaction temperatures in the first reaction zone and the second reaction zone are substantially the same. In such embodiments, the use of a heat exchanger is not required. Typically, however, the reaction temperature of the second reaction zone is less than the reaction temperature of the first reaction zone. A heat exchanger may lower the temperature of the stream leaving the first reaction zone to the appropriate temperature for a reaction to occur in the second reaction zone. Pressure maintained in second reaction zone 530 may be at a hydrocarbon partial pressure ranging from about 0.1 atmosphere (10 kPa) to about 20 atmospheres (2026 kPa). In an embodiment, partial pressure may range from above about 0.5 atmosphere (51 kPa) to about 10 atmospheres (1013 kPa).).

The second hydrocarbon stream may exit second reaction zone 530 via second conduit 550 and enter alkylation unit 140. At least a portion of a third hydrocarbon stream may be introduced into second conduit 550 via third conduit 560 to form a combined stream. The combined stream may be introduced into alkylation unit 140 via second conduit 550. At least a portion of the olefins in the combined stream may alkylate at least a portion of the aromatic hydrocarbons to produce alkyl aromatic hydrocarbons. In an embodiments a third hydrocarbon stream may be introduced directly into alkylation unit 140 through one or more points of entry.

The third hydrocarbon stream may be used to optimize the olefin concentration in alkylation unit 140 at a concentration sufficient to maximize monoalkylation of the aromatic hydrocarbon. In addition, the third hydrocarbon may optimize the ratio of linear to branched alkyl groups in the alkyl aromatic hydrocarbon, as has been described above. The third hydrocarbon stream nay be, but is not limited to, a hydrocarbon stream containing olefins, paraffins and/or hydrocarbon solvents.

In an embodiment, the third hydrocarbon stream includes a paraffin content of between about 50 percent and about 99 percent relative to the total hydrocarbon content. In certain embodiments, aparaffin content of the third hydrocarbon stream ranges between 60 percent and 90 percent relative to the total hydrocarbon content. In another embodiment, a paraffin content is greater than about 80 percent relative to the total hydrocarbon content.

In an embodiment, an olefin content of a third hydrocarbon stream ranges between about 1 percent and about 99 percent relative to the total hydrocarbon content. In certain embodiments, an olefin content of a third hydrocarbon stream may be between about 5 percent and about 15 percent relative to the total hydrocarbon content. In other embodiments, an olefin content of a third hydrocarbon stream may be greater than 80 percent relative to the total hydrocarbon stream.

Separation of at least a portion of unreacted olefins and at least a portion of paraffins from the alkyl aromatic hydrocarbons may be performed as previously described for System 100 depicted in FIG. 1. The alkylation reaction mixture stream may enter separator 160 via fourth conduit 570. In separator 160 at least two streams, an unreacted hydrocarbons stream and an alkyl aromatic hydrocarbons stream may be produced. At least a portion of the unreacted hydrocarbons stream may be separated into an aromatic hydrocarbons stream and a paraffins and unreacted olefins stream using generally known techniques (e.g., distillation, solid/liquid separation, absorption, solvent extraction). At least a portion of the separated aromatic hydrocarbons may be transferred to alkylation unit 140 via fifth conduit 580. At least a portion of the paraffins and unreacted olefins stream may be combined with other process streams, sent to other processing units and/or stored on site. The alkyl aromatic hydrocarbons product stream may be transported via sixth conduit 590 to be stored on site, sold commercially, transported off-site and/or utilized in other processing units.

At least a portion of the paraffins and unreacted olefins stream may be combined via seventh conduit 595 with the first hydrocarbon stream in first conduit 510, as depicted in System 500 of FIG. 6A, to produce a combined hydrocarbon stream. The combined hydrocarbon stream may enter first reaction zone 520 of dehydrogenation-isomerization unit 310 via first conduit 510. The combined hydrocarbon stream entering first reaction zone 520 continues the dehydrogenation-isomerization process and alkylation process to produce alkyl aromatic hydrocarbons. By recycling the paraffins and unreacted olefins stream, yield of product may be maximized. In an embodiment, a paraffins and unreacted olefins stream may directly enter dehydrogenation-isomerization unit 310 through one or more points of entry.

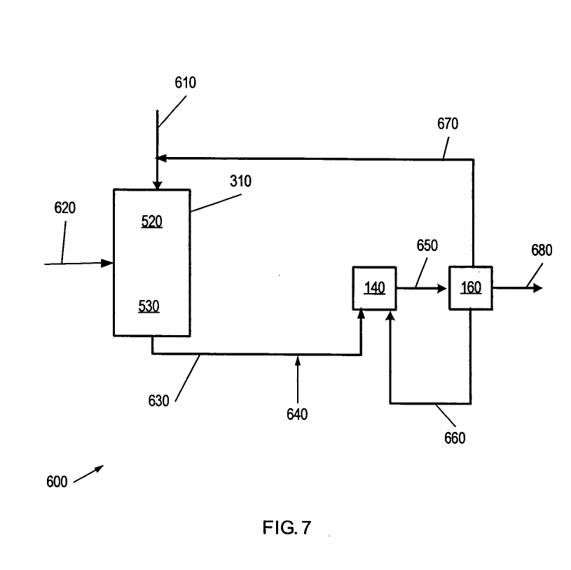

In an embodiment, a catalyst in dehydrogenation-isomerization unit may be used in a stacked bed configuration. A stacked bed configuration may allow for the use of one or more catalyst in the reactor. A separate catalyst for dehydrogenation of paraffins and then isomerization of olefins may enhance the selectivity of the catalysts and/or the process. System 600 depicted in FIG. 7 depicts a stacked bed configuration of dehydrogenation-isomerization unit 310. Operating conditions of the stacked bed configuration may be the same as for two-zone system described above for System 500 depicted in FIG. 6. The first hydrocarbon stream may enter the dehydrogenation zone 520 via first conduit 610.

The dehydrogenation catalyst used in the stacked bed configuration may have nonacidic properties. The term “nonacidic,” as used herein, refers to a catalyst that has very little skeletal isomerization activity. The dehydrogenation catalyst may include a Group VIII component, a Group IVA component, an alkali or alkaline earth component, a halogen component and a porous carrier material.

A Group VIII component may include, but is not limited to, platinum, palladium, iridium, rhodium, osmium, ruthenium or combinations thereof. In certain embodiments, a Group VIII component may be dispersed throughout the catalyst. The Group VIII component may range between about 0.01 weight percent to about 5 weight percent, calculated on an elemental basis, of the final catalytic composite. In certain embodiments, the dehydrogenation catalyst includes about 0.1 weight percent to about 1 weight percent platinum. The Group VIII component may be incorporated in the catalytic composite by generally known techniques (e.g., co-precipitation, co-gelation, ion exchange, impregnation, deposition from a vapor phase or from an atomic source) before, during and/or after incorporation of other catalytic components. In certain embodiments, a Group VIII component may be incorporated by impregnation of the carrier material with a solution or suspension of a decomposable compound of the Group VIII metal. In other embodiments, platinum may be added to a support by commingling the platinum with an aqueous solution of chloroplatinic acid. In other embodiments, nitric acid and/or other optional components may be added to the impregnating solution to further assist in dispersing or fixing the Group VIII component in the final catalyst composite.

A Group IVA component, may include, but is not limited to, germanium, tin, lead or combinations thereof. In some embodiments, a Group IVA component may exist within the catalyst in an oxidation state above that of the Group VIII component. The Group IVA component may be present as an oxide, combined with the carrier material, combined with the other catalytic components, or dispersed throughout the catalyst. In certain embodiments, a Group IVA component may range between about 0.01 weight percent to about 5 weight percent, calculated on an elemental basis, of the final catalyst composite. In some embodiments, a catalyst includes about 0.2 weight percent to about 2 weight percent tin.

A Group IVA component may be incorporated in the catalytic composite according to generally known techniques (e.g., co-precipitation, co-gelation, ion exchange, impregnation) before, during and/or after other catalytic components are incorporated. In certain embodiments, a tin component may be incorporated by co-gelation with the porous carrier material. In certain embodiments, tin may be incorporated in an alumina carrier material by mixing a soluble tin compound (e.g., stannous or stannic chloride) with an alumina hydrosol, adding a gelling agent (e.g., hexamethylenetetramine) and dropping the mixture into an oil bath to form spheres containing alumina and tin. In other embodiments, a germanium component may be impregnated into a carrier material with a solution or suspension of a decomposable compound of germanium (e.g., germanium tetrachloride dissolved in an alcohol). In another embodiment, a lead component may be impregnated from a solution of lead nitrate in water.

In certain embodiments, an alkali or alkaline earth component may include, but is not limited to, cesium, rubidium, potassium, sodium, lithium, barium, strontium, calcium and magnesium or mixtures of components thereof. An alkali or alkaline earth component may exist in the final catalytic composite in an oxidation state above that of the Group VIII component. The alkali or alkaline earth component may be present as an oxide, combined with the carrier material or combined with the other catalytic components.

In certain embodiments, an alkali or alkaline earth component may be dispersed throughout the catalytic composite. An alkali or alkaline earth component, in some embodiments, may range from about 0.01 weight percent to 15 weight percent, calculated on an elemental basis, of the final catalytic composite. In other embodiments, the catalyst includes about 1 weight percent to about 3 weight percent potassium. In certain embodiments, an atomic ratio of the alkali or alkaline earth component to the Group VIII component may be at least greater than about 10.

An alkali or alkaline earth component may be incorporated in the catalytic composite according to generally known techniques (e.g., co-precipitation, co-gelation, ion exchange or impregnation) before, during or after other catalytic components are incorporated. In certain embodiments, a potassium component may be impregnated into the carrier material with a solution of potassium nitrate. In some embodiments, an atomic ratio of alkali or alkaline earth component to Group VIII component may be at least about 10. In certain embodiments, an atomic ratio of the alkali or alkaline earth component to the Group VIII component may range from about 15 to about 25.

A porous carrier material may include a porous, absorptive support with high surface area of from about 25 m²/g to about 500 m²/g. The porous carrier material may have a melting point greater than the conditions utilized in the dehydrogenation zone. Examples of carrier materials include, but are not limited to, activated carbon, coke, charcoal, silica, silica gel, silicon carbide, synthetically prepared and/or naturally occurring clays and silicates, refractory inorganic oxides crystalline zeolitic aluminosilicates, naturally occurring or synthetically prepared mordenite and/or faujasite, spinels or combinations of thereof. In certain embodiments, a carrier material may be gamma- or eta-alumina. In some embodiments, clays and silicates may or may not be acid treated, (e.g., attapulgite, china clay, diatomaceous earth, fuller's earth, kaolin, kieselguhr, ceramics, porcelain, crushed firebrick, bauxite). Examples of refractory inorganic oxides include, but are not limited to, alumina, titanium dioxide, zirconium dioxide, chromium oxide, beryllium oxide, vanadium oxide, cerium oxide, hafnium oxide, zinc oxide, magnesia, boria, thoria, silica-alumina, silica-magnesia, chromia-alumina, alumina-boria, silica-zirconia) Zeolitic aluminosilcates may be, in some embodiments, either in the hydrogen form or in a form, which may be exchanged with metal cations. Examples of spinels include, but are not limited to, MgAl₂O₄, FeAl₂O₄, ZnAl₂O₄, CaAl₂O₄, and other like compounds having the formula MO—Al₂O₃ where M is a metal having a valence of 2.

In certain embodiments, an alumina carrier material may be prepared in any suitable manner from synthetic or naturally occurring raw materials. An alumina carrier may be formed in any desired shape (e.g., spheres, pills, cakes, extrudates, powders, granules). The alumina carrier may be utilized in any particle size. In certain embodiments, a sphere shape may be utilized. The particle may be about 1/16 inch in diameter. In certain embodiments, a particle diameter of less than about 1/32 inch may be utilized.

Alumina spheres may be prepared, in some embodiments, by converting aluminum metal into an alumina sol. An alumina sol may be prepared by reacting aluminum metal with a suitable peptizing acid and water. The resulting alumina sol and a gelling agent may be dropped into an oil bath to form spherical particles of an alumina gel. Alumina gel may be converted to gamma- or eta-alumina carrier material by methods generally known such as aging, drying and calcining.

In other embodiments, alumina cylinders may be prepared by mulling alumina powder with water and a suitable peptizing agent (e.g., nitric acid) to form an extrudable composition. The composition may then be extruded through a suitably sized die and cut to form extrudate particles. Other shapes of the alumina carrier material may also be prepared by conventional methods. After the alumina particles are shaped, they may be dried and calcined. The alumina carrier may be subjected to intermediate treatments during its preparation (e.g., washing with water or a solution of ammonium hydroxide).

The dehydrogenation catalytic composite may include a halogen component. The halogen component may include, but is not limited to, fluorine, chlorine, bromine, iodine or mixtures thereof. The halogen component may be present in a combined state with the porous carrier material. In certain embodiments, a halogen component may be dispersed throughout the catalytic composite. The halogen component may range from at least about 0.2 weight percent to about 15 weight percent, calculated on an elemental basis, of the final catalytic composite. In certain embodiments, the catalyst includes about 1 weight percent to about 3 weight percent chlorine.

The halogen component may be incorporated in the catalytic composite in any suitable manner. The incorporation of the halogen may be before preparation of the carrier material, during or after other catalytic components are incorporated. In certain embodiments, an alumina sol carrier may contain a halogen, which may contribute to at least some portion of the halogen content in the final catalyst composite. In some embodiments, a halogen component, or a portion thereof, may be added to the catalyst composite during the incorporation of the carrier material with other catalyst components (e.g., using chloroplatinic acid to impregnate the platinum component). In certain embodiments, a halogen component or a portion thereof may be added to the catalyst composite by contacting the catalyst with the halogen, or a compound, solution, suspension or dispersion containing the halogen, before other catalyst components are incorporated with the carrier material (e.g., hydrochloric acid). In other embodiments, a halogen component or a portion thereof may be added to the catalyst composite by contacting the catalyst with the halogen, or a compound, solution, suspension or dispersion containing the halogen, after other catalyst components are incorporated with the carrier material (e.g., hydrochloric acid). In certain embodiments, a halogen component or a portion thereof may be incorporated by contacting the dehydrogenation catalyst with a compound, solution, suspension or dispersion containing the halogen in a subsequent dehydrogenation catalyst regeneration step. In the regeneration step, carbon deposited on the dehydrogenation catalyst as coke during use of the catalyst in a hydrocarbon conversion process may be burned off the catalyst and the platinum group component on the catalyst may be redistributed to provide a regenerated catalyst with performance characteristics similar to the fresh catalyst. The halogen component may be added during the carbon burn step. In another embodiment, the halogen component may be added during the Group VIII component redistribution step (e.g., contacting the catalyst with a hydrogen chloride gas). In some embodiments, a halogen component may be added to the catalyst composite by adding the halogen or, a compound, solution, suspension or dispersion containing the halogen (e.g., propylene dichloride), to the hydrocarbon feed stream. In other embodiments, a halogen component may be added to the catalyst composite by adding the halogen or, a compound, solution, suspension or dispersion containing the halogen (e.g., propylene dichloride), to a recycle gas during operation of the dehydrogenation process.

In certain embodiments, a dehydrogenation catalyst composition may include at least about 0.2 weight percent, calculated on an elemental basis, of a halogen component. The halogen component in the catalyst may improve the activity of the catalyst for dehydrogenating hydrocarbons. In addition, the active halogen component may suppress carbon formation on the catalyst during use in a dehydrogenation process. An advantage of the catalyst composition may be that undesirable isomerization and/or cracking side reactions may be inhibited. In certain embodiments, a halogen content may increase the acidity of the catalyst. The acidity may be lowered by steaming the dehydrogenation catalyst to remove excess halogen from the dehydrogenation catalyst.

In some embodiments, a dehydrogenation catalyst may include a sulfur component ranging from about 0.01 weight percent to about 10 weight percent, calculated on an elemental basis, of the final catalytic composition. The sulfur component may be incorporated into the catalytic composite in any suitable manner. In certain embodiments, sulfur and/or a compound containing sulfur (e.g., hydrogen sulfide, a lower molecular weight mercaptan) may be contacted with the catalyst composition in the presence of hydrogen at a temperature ranging from about 10° C. to about 540° C. under anhydrous conditions. A hydrogen to sulfur ratio, in some embodiments, may be about 100.

The dehydrogenation catalyst, in some embodiments, may also contain other, additional components and/or mixtures of components that may improve catalyst activity, selectivity and/or stability. Examples of additional components include, but are not limited to, antimony, arsenic, beryllium, bismuth, cadmium, calcium, chromium, cobalt, copper, gallium, gold, indium, iron, lithium, manganese, molybdenum, nickel, rhenium, scandium, silver, tantalum, thallium, titanium, tungsten, uranium, zinc and/or zirconium. Additional components may be added in any suitable manner to the carrier material during or after its preparation. Additional components may be added in any suitable manner to the catalytic composite before catalytic components are incorporated. In other embodiments, additional components may be added during incorporation of catalytic components. In some embodiments, additional components may be added after incorporation of catalytic components. A description of a dehydrogenation catalyst may be found in U.S. Pat. No. 4,506,032 to Imai et al., entitled “Dehydrogenation Catalyst Composition,” which is incorporated by reference herein.

Referring back to FIG. 7, the process stream may pass into isomerization zone 530. In certain embodiments, a temperature decrease from dehydrogenation zone 520 to isomerization zone 530 may be necessary to prevent cracking of the first hydrocarbon stream as it enters the isomerization zone. Cool hydrogen gas may be introduced to dehydrogenation zone 520 via gas conduit 620, as depicted in FIG. 7 to control temperatures in dehydrogenation zone 520. In isomerization zone 530 at least a portion of the olefins in the process stream may be isomerized to branched olefins to produce a second hydrocarbon stream.

In certain embodiments, an isomerization catalyst may be the same as described for isomerization of olefins in System 100. A description of the isomerization catalyst may be found in U.S. Pat. No. 5,510,306 to Murray, entitled “Process For Isomerizing Linear Olefins to Isoolefins,” which is incorporated by reference herein. In some embodiments, about 0.01 weight percent to about 5 weight percent of a noble metal may be added to an isomerization catalyst used in a stacked bed configuration to increase the dehydrogenation activity of the zeolitic catalyst. When a noble metal such as platinum and/or palladium is added to the zeolitic catalyst, common metal incorporation methods known to those skilled in the art such as impregnation, noble metal ion exchange and co-mulling may be used to produce a working catalyst useful in dehydrogenation-isomerization of paraffins.

The second hydrocarbon stream may exit second reaction zone 530 and enter alkylation unit 140 via second conduit 630. At least a portion of a third hydrocarbon stream may be introduced into second conduit 630 via third conduit 640, as depicted in FIG. 7. The combined stream may be introduced into alkylation unit 140 via second conduit 630. At least a portion of the olefins in the combined stream may alkylate at least a portion of the aromatic hydrocarbons to produce alkyl aromatic hydrocarbons.

In an embodiment, the third hydrocarbon stream includes a paraffin content of between about 50 percent and about 99 percent relative to the total hydrocarbon content. In certain embodiments, a paraffin content of the third hydrocarbon stream ranges between 60 percent and 90 percent relative to the total hydrocarbon content. In another embodiment, a paraffin content is greater than about 80 percent relative to the total hydrocarbon content.

In an embodiment, an olefin content of a third hydrocarbon stream ranges between about 1 percent and about 99 percent relative to the total hydrocarbon content. In certain embodiments, an olefin content of a third hydrocarbon stream may be between about 5 percent and about 15 percent relative to the total hydrocarbon content. In other embodiments, an olefin content of a third hydrocarbon stream may be greater than 80 percent relative to the total hydrocarbon stream.

Separation of at least a portion of unreacted olefins and at least a portion of paraffins from the alkyl aromatic hydrocarbons may be performed as previously described for System 100 depicted in FIG. 1. The alkylation reaction mixture stream may enter separator 160 via fourth conduit 650. In separator 160 at least two streams, an unreacted hydrocarbons stream and an alkyl aromatic hydrocarbon stream may be produced. The unreacted hydrocarbons stream may be separated into an aromatic hydrocarbons stream and a paraffins and unreacted olefins stream using generally known techniques (e.g., distillation, solid/liquid separation, absorption, solvent extraction). Separated aromatic hydrocarbon may be transferred to alkylation unit 140 via fifth conduit 660. At least a portion of the paraffins and unreacted olefins stream may be combined with other process streams, sent to other processing units and/or stored on site via sixth conduit 670. The alkyl aromatic hydrocarbon product stream may be transported via seventh conduit 680 to be stored on site, sold commercially, transported off-site and/or utilized in other processing units.

As depicted in FIG. 7, at least a portion of the paraffins and unreacted olefins stream, via sixth conduit 670, may be introduced into first conduit 610 to produce a combined stream. The combined stream may be introduced into dehydrogenation-isomerization unit 310 via first conduit 610 and at least a portion of the paraffins in the combined stream may be dehydrogenated and isomerized to branched olefins. In an embodiment, at least a portion of an paraffins and unreacted olefins stream may be introduced directly into dehydrogenation-isomerization unit 310 via one or more points of entry. In certain embodiments, one or more combined process streams and at least a portion of an paraffins and unreacted olefins stream may be introduced directly into dehydrogenation-isomerization unit 310 through one or more points of entry.

In certain embodiments, a Fischer-Tropsch feed stream may contain olefins and paraffins of low carbon number (e.g., 4, 5, 6, 7, 8). Typically, a low carbon number feed stream may be sold as fuel, sent to waste and/or recycled to other processing units. The low carbon number of the olefins and paraffins makes the stream less useful for the production of detergents. Typically detergents are made from olefins having a carbon number greater than 8. Conversion of the olefins in the feed stream to branched olefins with higher average carbon number (e.g., 8 to 16) may result in a more commercially valuable use of a low carbon number feed stream (e.g., processed to produce detergents and/or surfactants). An amount and type of branching of the alkyl group may increase the value of the feed stream. Processes to convert low carbon number olefins to higher carbon number olefins are described.

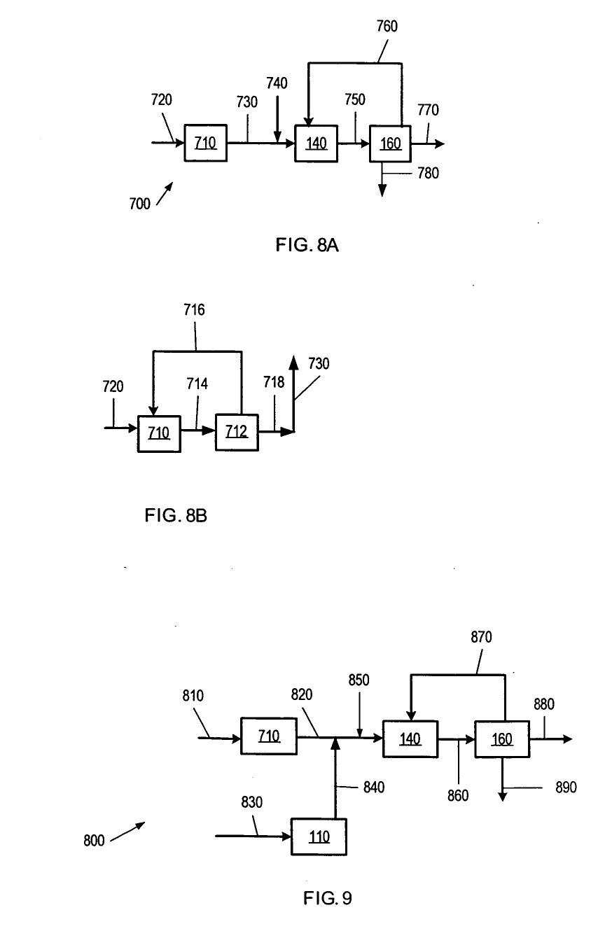

Referring to System 700 depicted in FIG. 8A, a first hydrocarbon stream, including olefins and paraffins may be introduced into dimerization unit 710 via first conduit 720. In certain embodiments, hydrocarbons in the first hydrocarbon stream may have an average carbon number from 4 to 8. In other embodiments, hydrocarbons in the first hydrocarbon stream may have an average carbon number from 4 to 6. The first hydrocarbon stream may, in some embodiments, be derived from a Fischer-Tropsch process.

In certain embodiments, a dimerization unit may have several points of entry to accommodate different process streams. Process streams may be from other processing units and/or storage units. Examples of process streams include, but are not limited to, a diluent hydrocarbon stream, and other hydrocarbon streams that include olefins and paraffins derived from other processes. As used herein, “entry into the dimerization unit” refers to entry of process streams into the dimerization unit through one or more entry points.

In dimerization unit 710, at least a portion of the olefins may be dimerized. At least a portion of the dimerized olefins exit dimerization unit 710 as a second hydrocarbon stream via second conduit 730. Depending on the choice of catalyst, the resulting dimer may be branched. Branches of the olefin produced in dimerization unit 710 may include methyl, ethyl and/or longer carbon chains. In an embodiment, dimerized olefins may contain greater than about 50 percent methyl branches. In some embodiments, dimerized olefins may contain greater than about 90 percent methyl branches. Dimerized olefins may be separated through generally known techniques. One such technique is fractional distillation. At least a portion of the paraffins and unreacted olefins may be separated and recycled back to the dimerization unit and/or sent to other processing units.

A dimerization catalyst used in dimerization unit 710 may be a homogeneous catalyst. In some embodiments, a dimerization catalyst may be a heterogeneous catalyst. In certain embodiments, a dimerization catalyst used in dimerization unit 710 may be a catalyst that includes oxides of Group III, Group IVA, Group IVB, Group VIIIA, and/or combinations thereof. Examples oxides include, but are not limited to, nickel oxide, silicon dioxide, titanium dioxide, aluminum oxide and/or zirconium dioxide. The catalyst may include an amorphous nickel oxide (NiO) present as a dispersed substantial monolayer on the surfaces of a silica (SiO₂) support. The silica support may also include on the surface minor amounts of an oxide of aluminum, gallium and/or indium such that the ratio of nickel oxide to metal oxide present in the catalyst is within the range of from about 4:1 to about 100:1. The dimerization catalyst may be prepared by precipitating a water insoluble nickel salt onto the surface of a silica support, which has been impregnated with the metal oxide. Alternatively, a dimerization catalyst may be prepared by precipitating a water insoluble nickel salt onto a silica-alumina support, which has been dealuminized such that the resulting nickel oxide/alumina ratio falls within the ranges set forth above. The catalyst may be activated by calcination in the presence of oxygen at a temperature with the range of from about 300° C. to about 700° C. or, in some embodiments, a temperature range from about 500° C. to about 600° C.

A silica support surface area may be within the range of from about 100 m²/g to about 450 m²/g. In an embodiment, a silica surface area may be within the range from about 200 m²/g to about 400 m 2/g. A nickel oxide content dispersed in the silica support may range from about 7 percent to about 70 percent by weight. In certain embodiments, a nickel oxide content may be from about 20 percent to about 50 percent by weight, depending on the surface area of the particular support utilized in preparing a catalyst. For a silica support having a surface area of about 300 m²/g, a nickel oxide content may, in some embodiments, ranges from about 21 percent to about 35 percent by weight. A nickel oxide content may, in other embodiments, be about 28 percent by weight.

A silica support may be in dry granular form prior to precipitation of the nickel oxide precursor compound on the support surfaces. In some embodiments, a silica support may be in a hydrogel form prior to precipitation of the nickel oxide precursor compound on the surport surfaces. Silica hydrogel may be prepared by mixing a water-soluble silicate, (e.g., a sodium or potassium silicate) with a mineral acid then washed with water to remove water-soluble ions. A silica hydrogel maybe partially dried before use. In some embodiments, a silica hydrogel may be completely dried before use.

A nickel oxide precursor may include a water-insoluble nickel salt (e.g., nickel carbonate, nickel phosphate, nickel nitrate or nickel hydroxide). The water-insoluble nickel salt may be generated in-situ by forming an aqueous mixture of the silica gel and a water-soluble nickel salt. The nickel salt may include, but is not limited to, nickel nitrate, nickel sulfonate, nickel carbonylate, nickel halide. A base may be added to the aqueous mixture to induce precipitation of the water-insoluble nickel salt. The water-insoluble nickel salt, which may be formed, may be precipitated in finely divided form within the interstices and on the surface of the silica support. The treated silica support may then be recovered, washed several times and dried.

A second component in the catalyst may be a trivalent metal oxide, which may include, but is not limited to, aluminum, gallium and indium and/or combinations thereof. Although the nickel oxide and/or silica catalyst described above may be active for olefin dimerization, it may deactivate quickly, probably as a consequence of the formation of large oligomers which remain attached to the surface and act as coke precursors. A presence of a small amount of the trivalent metal oxide within the catalyst yields acid sites. Acid sites may promote catalytic activity without promoting unwanted and/or excessive oligomer formation.

A trivalent metal oxide may be incorporated into the silica support by any suitable technique (e.g., the precipitation method described above or by direct impregnation in the form of a water-soluble salt). In an embodiment, a trivalent metal oxide may be impregnated into the silica support as an aqueous solution by the addition of a water-soluble salt. The water-soluble metal salt may include, but is not limited to, metal nitrates, metal chlorides and/or metal sulfates. Once impregnated with a metal salt, the silica support may be dried and calcinated to reduce the metal salt to an oxide form. The resulting silica support activated with the trivalent oxide may then be further treated to incorporate the nickel oxide layer onto the silica-trivalent metal oxide support.

In some embodiments, a silica-trivalent metal oxide supports may include silica/alumina, silica/gallia and/or silica/india gel. In certain embodiments, content of a metal oxide (e.g., alumina) present in the support may be low in comparison with the content of nickel oxide. Therefore, dealuminization of the silica/alumina gel of relatively high alumina content (e.g., above about 5 percent by weight) may be necessary to reduce the content of alumina. Dealuminization may be accomplished by generally known techniques (e.g., extraction of the aluminum with an organic or inorganic acid). Acids include, but are not limited to, nitric acid, sulfuric acid, hydrochloric acid, chloroacetic acid or ethylene diamine tetraacetic acid. Extraction of the support may be accomplished by adding an acid to an aqueous dispersion of the alumino silicate followed by stirring, decantation and washing with water. The process may be repeated one or more times until a desired alumina content is achieved. The solids may then dried, calcined and further treated as described above to incorporate the nickel oxide layer onto the silica/alumina support.

The content of trivalent metal oxide with respect to the content of the nickel oxide present in the silica support may be significant, in some embodiments, in order to achieve improved results in terms of dimer yield and minimum average methyl branches present in the dimer product. In certain embodiments, when the content of trivalent metal oxide is too low, e.g., above a nickel oxide to trivalent metal oxide ratio of about 100 to 1, then the yield of dimer decreases and the catalyst may tend to deactivate quickly. In certain embodiments, when the content of trivalent metal oxide is too high, e.g., below a nickel oxide to trivalent metal oxide ratio of about 4 to 1, then the yield of dimer may tend to decreases and the average content of methyl branches in the dimer product may tend to increase. In certain embodiments, content of trivalent metal oxide may be such that the ratio of nickel oxide to trivalent metal oxide falls within the range of from about 4:1 to about 30:1. In other embodiments, content of trivalent metal oxide may be such that the ratio of nickel oxide to trivalent metal oxide is between about 5:1 to about 20:1. In certain embodiments, a ratio of nickel oxide to trivalent metal oxide may be between about 8:1 to about 15:1.

In certain embodiments, a dimerization catalyst may contain from about 21 percent to about 35 percent by weight of nickel oxide and about 1 percent to about 5 percent by weight of trivalent metal oxide, based on the total weight of nickel oxide, trivalent metal oxide and silica. In certain embodiments, a dimerization catalyst may include from about 1.5 percent to about 4 percent by weight trivalent metal oxide based on the total weight of nickel oxide, trivalent metal oxide and silica.

Preparation of dimerization catalysts are described in U.S. Pat. No. 5,849,972 to Vicari et al., entitled “Oligomerization Of Olefins To Highly Linear Oligomers, and Catalyst For This Purpose,” and U.S. Pat. No. 5,169,824 to Saleh et al., entitled “Catalyst Comprising Amorphous NiO On Silica/Alumina Support”, both of which are fully incorporated herein by reference.

Conversion of olefins in the first hydrocarbon feed stream to dimers in dimerization unit 710 may be carried out as a batch, continuous (e.g. using a fixed bed), semi-batch or multi-step process. In a batch process, the catalyst may be slurried with the first hydrocarbon feed stream. Temperature conditions for the dimerization reaction may range from about 120° C. to about 200° C. In an embodiment, a reaction temperature may range from about 150° C. to about 165° C. Reaction temperatures may be controlled with evaporative cooling. For example, the evaporation of lighter hydrocarbon fractions from the reaction mixture may be used to control a reaction temperature.

Produced dimerized olefins may exit the dimerization unit in a second hydrocarbon stream via second conduit 730. The second hydrocarbon stream may be transported to other processing units (e.g., separation unit, an alkylation unit, hydroformylation unit). In some embodiments, the second hydrocarbon stream may include olefins with an average carbon number between 8 and 16. In other embodiments, the second hydrocarbon stream may include olefins with an average carbon number between 8 and 12. The second hydrocarbon stream may include, in some embodiments, an olefin content of greater than 50 percent by weight.

Produced dimerized olefins may be separated, if desired, from the reaction mixture through generally known techniques (e.g., fractional distillation). In an embodiment, at least a portion of second hydrocarbon stream may exit dimerization unit 710 and enter separation unit 712 via conduit 714 as depicted in FIG. 8B. In separation unit 712 the reaction mixture may be separated into a produced dimerized olefins stream and a paraffins and unreacted olefins stream through fractional distillation. As used herein, “fractional distillation” refers to distillation of liquids and subsequent collection of fractions of liquids determined by boiling point. The paraffins and unreacted olefins stream may contain hydrocarbons with a carbon number less than 8. At least a portion of the paraffins and unreacted olefins stream may be introduced into dimerization unit 710 via conduit 716. In other embodiments, the paraffins and unreacted olefins stream may be combined with a process stream upstream of the dehydrogenation unit. Produced dimerized olefins stream may exit separation unit 712 and be introduced into second conduit 730 via conduit 718.

Referring back to System 700, depicted in FIG. 8A, at least a portion of the second hydrocarbon stream may be introduced into alkylation unit 140 via second conduit 730. At least a portion of a third hydrocarbon stream may be introduced into second conduit 730 via third conduit 740 to produce a combined hydrocarbon stream. The combined stream may enter alkylation unit 140. At least a portion of the olefins in the combined stream may alkylate the aromatic hydrocarbons in alkylation unit 140 to produce an alkylation reaction mixture stream. Alkylation may be performed under conditions described for System 100 depicted in FIG. 1.

As previously described for System 100, at least a portion of the third hydrocarbon stream may be used to regulate the olefin concentration in alkylation unit 140 at a concentration sufficient to maximize monoalkylation of the aromatic hydrocarbon. In addition, the third hydrocarbon stream may optimize the ratio of linear to branched alkyl aromatic hydrocarbons. The third hydrocarbon stream may be, but is not limited to, a hydrocarbon stream containing olefins, paraffins and/or hydrocarbon solvents.

In an embodiment, the third hydrocarbon stream includes a paraffin content of between about 50 percent and about 99 percent relative to the total hydrocarbon content. In certain embodiments, a paraffin content of the third hydrocarbon stream ranges between 60 percent and 90 percent relative to the total hydrocarbon content. In another embodiment, a paraffin content is greater than about 80 percent relative to the total hydrocarbon content.

In an embodiment, an olefin content of a third hydrocarbon stream ranges between about 1 percent and about 99 percent relative to the total hydrocarbon content. In certain embodiments, an olefin content of a third hydrocarbon stream may be between about 5 percent and about 15 percent relative to the total hydrocarbon content. In other embodiments, an olefin content of a third hydrocarbon stream may be greater than 80 percent relative to the total hydrocarbon stream.

The alkylation reaction mixture stream may enter separator 160 via fourth conduit 750. In separator 160 at least two streams, an unreacted hydrocarbons stream and an alkyl aromatic hydrocarbons stream may be produced. Methods used for separation, in certain embodiments, may be the same as those described for System 100. The unreacted hydrocarbons stream may be separated into an aromatic hydrocarbons stream and a paraffins and unreacted olefins stream using generally known techniques (e.g., distillation, solid/liquid separation, absorption, solvent extraction). The separated aromatic hydrocarbons stream may exit separator 160 and be recycled to alkylation unit 140 via fifth conduit 760. The alkyl aromatic hydrocarbons product stream may exit separator 160 and be transported via sixth conduit 770 to be stored on site, sold commercially, transported off-site, and/or utilized in other processing units. At least a portion of the paraffins and unreacted olefins stream may exit separator 160 and be combined with other process streams, sent to other processing units and/or be stored on site via seventh conduit 780. In certain embodiments, the paraffins and unreacted olefins stream may be further separated into a hydrocarbons stream including paraffins and unreacted olefins with a carbon number less than 8. The hydrocarbon stream including paraffins and unreacted olefins with a carbon number less than 8 may be introduced upstream of and/or into dimerization unit 710.

In certain embodiments, a hydrocarbon-stream from a dimerization unit may be combined with a hydrocarbon stream from an isomerization unit to produce a combined stream. The combined stream may be introduced into an alkylation unit. Combining streams from the two units may result in a more economically valuable process to produce alkyl aromatic hydrocarbons. Referring to System 800, depicted in FIG. 9, a first hydrocarbon stream, that includes paraffins and olefins may enter dimerization unit 710 via first conduit 810. The first hydrocarbon stream, in an embodiment, may be produced from a Fischer-Tropsch process. In certain embodiments, an average carbon number of the hydrocarbons in the first hydrocarbon stream may range from 4 to 8 or, in some embodiments, 4 to 6.

In dimerization unit 710, at least a portion of the olefins may be dimerized as previously described for System 700. At least a portion of the dimerized olefins exit dimerization unit 710 as a second hydrocarbon stream. Depending on the choice of catalyst, the resulting dimer may be branched. Branches of the olefin produced in dimerization unit 710 may include methyl, ethyl and/or longer carbon chains. In an embodiment, produced dimerized olefin may contain greater than about 50 percent methyl branches. In some embodiments, produced dimerized olefins may contain greater than about 90 percent methyl branches. The average carbon number of the resulting produced dimerized olefins may range between 8 and 16, or some embodiments, 8 and 12. Produced dimerized olefins may be separated from the reactor product through generally known techniques (e.g., fractional distillation). In an embodiment, produced dimerized olefins may be separated from the reaction mixture and processed as previously described in FIG. 8B. Analysis of the composition and branching of the dimerized olefins may be performed using ¹H NMR techniques. The second hydrocarbon stream may be transferred to other processing units, (e.g., alkylation units, hydroformylation units) or to storage units through a conduit.

At least a portion of the second hydrocarbon stream may exit dimerization unit 710 and enter alkylation unit 140 via second conduit 820. A fourth hydrocarbon stream may be introduced directly into alkylation unit 140 through one or more alkylation unit ports. Referring to System 700 in FIG. 9, at least a portion of a fourth hydrocarbon stream may be introduced into second conduit 820 via fourth conduit 840 upstream of alkylation unit 140 to produce a combined stream. The fourth hydrocarbon stream may be a stream exiting from isomerization unit 110.

Isomerization unit 110 may be fed by a third hydrocarbon stream containing paraffins and unreacted olefins via a third conduit 830. In isomerization unit 110, at least a portion of the olefins in the third hydrocarbon stream may be isomerized to branched olefins to produce the fourth hydrocarbon stream using process conditions previously described for System 100. The fourth hydrocarbon stream may be combined with the second hydrocarbon stream in second conduit 820 as depicted in FIG. 9. In certain embodiments, hydrocarbons in the fourth hydrocarbon stream may have an average carbon number range between 8 and 16 or, in some embodiments, 10 to 13.

In an embodiment, a fifth hydrocarbon stream may be introduced into alkylation unit 140 through one or more alkylation ports. In certain embodiments, at least a portion of a fifth hydrocarbon stream may be introduced into second conduit 820 upstream of alkylation unit 140 via fifth conduit 850 to produce a combined stream. The combined stream may enter alkylation unit 140 and at least a portion of the olefins in the combined stream may alkylate aromatic hydrocarbons to produce alkyl aromatic hydrocarbons. Alkylation reaction conditions may be the same as previously described for System 100. The resulting alkylation product may be branched alkyl aromatic hydrocarbons.

The fourth and/or fifth hydrocarbon stream may be used to regulate the olefin concentration in alkylation unit 140 at a concentration sufficient to maximize monoalkylation of the aromatic hydrocarbon and the amount of alkyl branching in the product. The fifth hydrocarbon stream may be, but is not limited to, a hydrocarbon stream containing olefin, paraffins and/or hydrocarbon solvents. In an embodiment, a paraffin content of the fifth hydrocarbon stream may be between greater than about 50 percent and less than about 99 percent relative to the total hydrocarbon content. In certain embodiments, a paraffin content of the fifth hydrocarbon stream may be between. 60 and 90 percent relative to the total hydrocarbon content. In another embodiment, a paraffin content may be greater than about 80 percent relative to the total hydrocarbon content.

In an embodiment, an olefin content of a fifth hydrocarbon stream ranges between about 1 percent and about 99 percent relative to the total hydrocarbon content. In certain embodiments, an olefin content of a fifth hydrocarbon stream may be between about 5 percent and about 15 percent relative to the total hydrocarbon content. In other embodiments, am olefin content of a fifth hydrocarbon stream may be greater than 80 percent relative to the total hydrocarbon stream.

Referring to System 800, depicted in FIG. 9, a combined stream, which may include, but is not limited to, a second hydrocarbon stream, a fourth hydrocarbon stream, a fifth hydrocarbon stream and/or combinations thereof, may be introduced into alkylation unit 140 via second conduit 820. An advantage of combining the streams may be that overall production of alkyl aromatic hydrocarbons may be increased with fewer throughputs. At least a portion of the olefins in the combined stream may alkylate aromatic hydrocarbons under conditions previously described for System 100 to produce alkyl aromatic hydrocarbons.

The alkylation reaction mixture stream may enter separator 160 via sixth conduit 860. Separation of the alkyl aromatic hydrocarbon product from at least a portion of the paraffins and at least a portion of unreacted olefins may be performed in separator 160. Separation may produce at least two streams, an unreacted hydrocarbons stream and an alkyl aromatic hydrocarbon product stream. At least a portion of the unreacted hydrocarbons stream may be separated into an aromatic hydrocarbons stream and a paraffins and unreacted olefins stream using generally known techniques (e.g., distillation, solid/liquid separation, absorption, solvent extraction). At least a portion of the separated aromatic stream may exit separator 160 and be recycled to alkylation unit 140 via seventh conduit 870. The resulting alkyl aromatic hydrocarbon product stream may exit separator 160 and be transported via a eighth conduit 880 to be stored on site, sold commercially, transported off-site, and/or utilized in other processing units.

At least a portion of the paraffins and unreacted olefins stream may exit separator 160 and be transported via ninth conduit 870 to another processing unit, and/or storage vessel. In certain embodiments, the paraffins and unreacted olefins stream may be further separated into a hydrocarbons stream including paraffins and unreacted olefins with a carbon number less than 8. The hydrocarbon stream including paraffins and unreacted olefins with a carbon number less than 8 may be introduced upstream of and/or into dimerization unit 710.

In certain embodiments, the first hydrocarbon stream may contain unwanted compounds (e.g., oxygenates, dienes) that may reduce catalyst selectivity in processes used to produce alkyl aromatic hydrocarbons. Removal of the unwanted compounds may be performed by hydrogenation of the first hydrocarbon stream. Hydrogenation of the first hydrocarbon stream, in certain embodiments, may produce a hydrocarbon stream that includes greater than about 90 percent paraffins. The hydrogenated hydrocarbon stream may be dehydrogenated to produce an olefinic stream. A dehydrogenation catalyst may control the position of the olefin double bond. In certain embodiments, an olefinic hydrocarbon stream may include olefins in which greater than 70 percent of the olefins are alpha-olefins of a linear carbon skeletal structure. In other embodiments, an olefinic hydrocarbon stream may include olefins in which 50 percent or more of the olefin molecules present may be internal olefins.

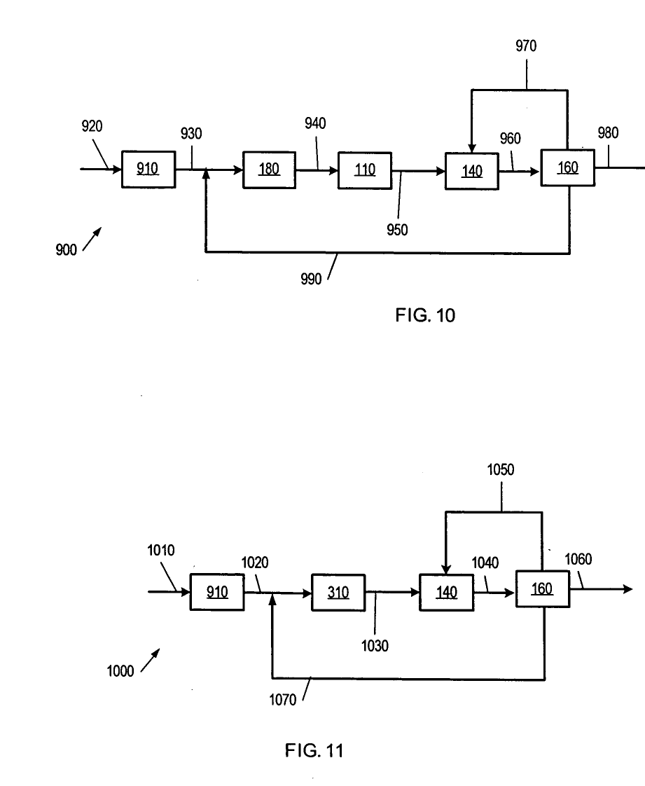

A first hydrocarbon stream may be introduced into hydrogenation unit 910 via first conduit 920 as depicted for System 900 in FIG. 10. In certain embodiments, hydrocarbons in the first hydrocarbon stream may have an average carbon number from 7 to 16. In other embodiments, hydrocarbons in the first hydrocarbon stream may have an average carbon number from 10 to 13. The first hydrocarbon stream includes olefins and paraffins. In hydrogenation unit 910, at least a portion of the olefins in the first hydrocarbon stream may be hydrogenated to paraffins to produce a second hydrocarbon stream.

Reaction conditions may be controlled to hydrogenate olefins, dienes and remove oxygenates using generally known techniques. Operating temperatures of hydrogenation unit 910 may range between about 100° C. and about 300° C. In some embodiments, an operating temperature may range between about 150° C. and about 275° C.; In other embodiments, an operating temperature may range between about 175° C. and 250° C. Operating pressures may range from 5 atmospheres (506 kPa) to about 150 atmospheres (15198 kPa). In some embodiments, operating pressures may range from 10 atmospheres psi (1013 kPa) to about 50 atmospheres (5065 kPa).

Hydrogenation may be carried out using any type of catalyst bed arrangement (e.g., fluidized bed, moving bed, slurry phase bed, a fixed bed). In certain embodiments, a fixed bed arrangement may be used. In a fixed bed system, hydrogen may be supplied to the hydrogenation stage at a gas hourly space velocity in the range of from about 100 normal liter gas/liter catalyst/hour (NL/L/hr) to about 1000 NL/L/hr. In some embodiments, hydrogen may be supplied at a gas hourly space velocity in the range of from about 250 NL/L/hr to 5000 NL/L/hr. “Gas space velocity as expressed in units of normal liter of gas/liter of catalyst/hour” as used herein, refers to the volume of a gas in liters at standard conditions of 0° C. and 760 mm Hg.

Hydrogenation catalysts are well generally known and are commercially available in a large variety of compositions. In some embodiments, a hydrogenation catalyst may include one or more metals from Groups VIB and VII of the periodic Table of the Elements. In certain embodiments, metals may include, but are not limited to molybdenum, tungsten, cobalt, nickel, ruthenium, iridium osmium, platinum and palladium. A hydrogenation catalyst may include a refractory oxide or a silicate as a binder.

Hydrogenation reaction conditions and catalysts are described in European Patent No. 0 583 836 to Eilers et al., entitled “Process For The Preparation of Hydrocarbon Fuels;” European Patent No. 0 668 342 to Eilers et al., entitled “Lubricating Base Oil Preparation Process” and U.S. Pat. No. 5,371,308 to Gosselink et al., entitled “Process For The Preparation Of Lower Olefins;” all of which are incorporated by reference herein.

At least a portion of the second hydrocarbon stream may exit hydrogenation unit 910 and enter dehydrogenation unit 180 via second conduit 930. At least a portion of the unreacted paraffins in the second hydrocarbon stream may be dehydrogenated using process conditions as previously described for System 100, At least a portion of the resulting olefinic hydrocarbon stream and at least a portion of the unreacted hydrocarbons in the second hydrocarbon stream may form a third hydrocarbon stream.

At least a portion of the third hydrocarbon steam may exit the dehydrogenation unit and enter isomerization unit 110 via third conduit 940. Conditions of the olefin isomerization may be controlled, as previously described in System 100, such that the number of carbon atoms in the olefin before and after isomerization is substantially the same. At least a portion of the olefins in the combined stream may be isomerized to branched olefins in isomerization unit 110 to produce a fourth hydrocarbon stream.

The fourth hydrocarbon stream may exit isomerization unit 110 and be introduced into alkylation unit 140 via fourth conduit 950 as depicted in FIG. 10. The fourth hydrocarbon stream includes branched olefins. In certain embodiments, an average carbon number of hydrocarbons in the fourth hydrocarbon stream may range from 7 to 16. In other embodiments, an average number of hydrocarbons in the fourth hydrocarbon stream may range from 10 to 13. At least a portion of the olefins in the fourth hydrocarbon stream may alkylate at least a portion of aromatic hydrocarbons using process conditions as previously described for System 100.

In certain embodiments, it may be desirable to adjust the olefin and paraffin concentration entering isomerization unit 110 and/or alkylation unit 140 depending on the source of the olefin stream as previously described for System 200 in FIG. 3. A paraffinic stream may be added to a process stream that contains greater than about 50 percent mono-olefins upstream of an alkylation unit and/or isomerization unit to produce a process stream that is less than about 50 percent mono-olefins. In some embodiments, a fifth hydrocarbon stream containing about 20 percent linear olefins and 80 percent paraffins may be added to a process stream containing primarily branched olefins upstream of an alkylation unit. Alkylation of aromatic hydrocarbons with the combined stream may result in a mixed stream containing branched and linear alkyl aromatic hydrocarbons.

In an embodiment, a fifth hydrocarbon stream may be added to process stream upstream of the alkylation unit and/or isomerization unit that contains an olefin content of between about 1 percent and about 99 percent relative to the total hydrocarbon content. In certain embodiments, an olefin content of the fifth hydrocarbon stream may be between about 5 percent and about 15 percent relative to the total hydrocarbon content. In other embodiments, an olefin content of the fifth hydrocarbon stream may be greater than 80 percent relative to the total hydrocarbon content.

The alkylation reaction mixture stream may enter separator 160 via fifth conduit 960. Separation of at least a portion of paraffins and at least a portion of olefins from the alkylation reaction mixture may be accomplished as previously described for System 100. At least two streams may be produced, an unreacted hydrocarbons stream and an alkyl aromatic hydrocarbons product stream. At least a portion of the unreacted hydrocarbons stream may be separated into an aromatic hydrocarbons stream and a paraffins and unreacted olefins stream using generally known techniques (e.g., distillation, solid/liquid separation, absorption, solvent extraction). At least a portion of the separated aromatic hydrocarbons stream may exit separator 160 and be recycled to alkylation unit 140 via sixth conduit 970. The alkyl aromatic hydrocarbon product stream may be transferred through seventh conduit 980 to be stored on site, sold commercially, transported off-site, and/or utilized in other processing units (e.g., sulfonation unit).

At least a portion of the paraffins and unreacted olefins stream may be combined with the second hydrocarbon stream in second conduit 930 upstream of dehydrogenation unit 180 via eighth conduit 990 to produce a combined stream. The combined stream may be introduced into dehydrogenation unit 180 and at least a portion of the paraffins in the combined stream may be dehydrogenated to olefins. In an embodiment, a paraffins and unreacted olefins stream may be introduced directly into dehydrogenation unit 180.

Referring to System 1000 as depicted in FIG. 11, a first hydrocarbon stream may be introduced into hydrogenation unit 910 via first conduit 1010. The first hydrocarbon stream includes olefins and paraffins. In hydrogenation unit 910, at least a portion of the olefins in the first hydrocarbon stream may be hydrogenated to paraffins to produce a second hydrocarbon stream. Reaction conditions in the hydrogenation unit 910, may be the same as previously described for System 900.

At least a portion of the second hydrocarbon stream may enter dehydrogenation-isomerization unit 310 via second conduit 1020. At least a portion of the paraffins in the second hydrocarbon stream may be dehydrogenated to olefins. At least a portion of the resulting olefins and at least a portion of the olefins that were already present in the feed stream may be isomerized to produce a third hydrocarbon stream. Process conditions used in dehydrogenation-isomerization unit 310 may be the same as previously described for Systems 300, 400, 500 and 600. At least a portion of the resulting olefinic hydrocarbon stream and at least a portion of the unreacted hydrocarbons in the second hydrocarbon stream may form a third hydrocarbon stream.

The third hydrocarbon stream may exit dehydrogenation-isomerization unit 310 and be introduced into alkylation unit 140 via third conduit 1030 as depicted in FIG. 11. The third hydrocarbon stream includes branched olefins. In certain embodiments, an average carbon number of hydrocarbons in the third hydrocarbon stream may range from 7 to 16. In other embodiments, an average number of hydrocarbons in the third hydrocarbon stream may range from 10 to 13. At least a portion of the olefins in the third hydrocarbon stream may alkylate at least a portion of aromatic hydrocarbons using process conditions as previously described for System 100.

In certain embodiments, it may be desirable to adjust the olefin and paraffin concentration in alkylation unit 140 depending on the source of the olefin stream as previously described for System 400 in FIG. 5. A paraffinic stream may be added to a process stream that contains greater than about 50 percent mono-olefins upstream of an alkylation unit to produce a process stream that is less than about 50 percent mono-olefins. In some embodiments, a fourth hydrocarbon stream containing about 20 percent linear olefins and 80 percent paraffins may be added to a process stream containing primarily branched olefins upstream of an alkylation unit. Alkylation of aromatic hydrocarbons with the combined stream may result in a mixed stream containing branched and linear alkyl aromatic hydrocarbons.

In an embodiment, a fourth hydrocarbon stream may be added to process stream upstream of the alkylation unit that contains an olefin content of between about 1 percent and about 99 percent relative to the total hydrocarbon content. In certain embodiments, an olefin content of the fourth hydrocarbon stream may be between about 5 percent and about 15 percent relative to the total hydrocarbon content. In other embodiments, an olefin content of the fourth hydrocarbon stream may be greater than 80 percent relative to the total hydrocarbon content.

The alkylation reaction mixture stream may enter separator 160 via fourth conduit 1040. Separation of at least a portion of paraffins and at least a portion of olefins from the alkylation reaction mixture may be accomplished as previously described for System 100. At least two streams may be produced, an unreacted hydrocarbons stream and an alkyl aromatic hydrocarbons product stream. At least a portion of the unreacted hydrocarbons stream may be separated into an aromatic hydrocarbons stream and a paraffins and unreacted olefins stream using generally known techniques (e.g., distillation, solid/liquid separation, absorption, solvent extraction). At least a portion of the separated aromatic hydrocarbons stream may exit separator 160 and be recycled to alkylation unit 140 via fifth conduit 1050. The alkyl aromatic hydrocarbons product stream may be transferred through sixth conduit 1060 to be stored on site, sold commercially, transported off-site, and/or utilized in other processing units (e.g., sulfonation unit).

At least a portion of the paraffins and unreacted olefins stream may be combined with the second hydrocarbon stream in second conduit 1020 upstream of dehydrogenation-isomerization unit 310 to produce a combined stream via seventh conduit 1070. The combined stream may be introduced into dehydrogenation-isomerization unit 310 and at least a portion of the paraffins in the combined stream may be dehydrogenated to olefins. The resulting olefins may be isomerized to branched olefins. In an embodiment, a combined paraffins and unreacted olefins stream may be introduced directly into dehydrogenation-isomerization unit 310.

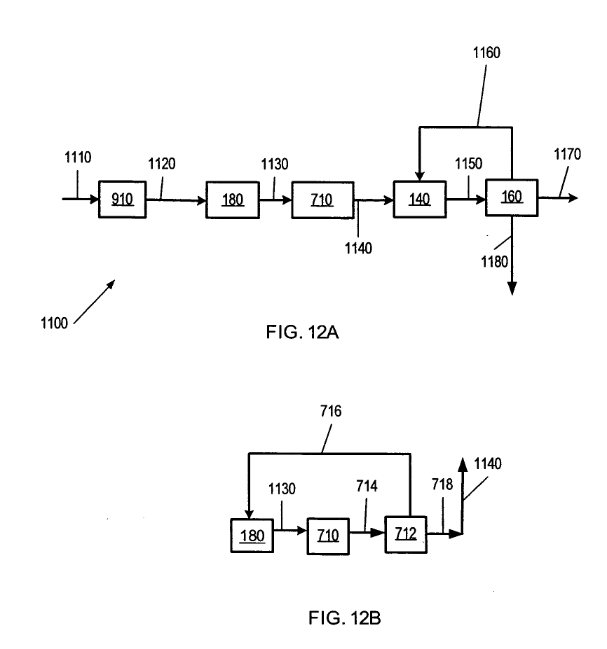

Referring to System 1100 as depicted in FIG. 12A, a first hydrocarbon stream may be introduced into hydrogenation unit 910 via first conduit 1110. The first hydrocarbon stream includes olefins and paraffins. In hydrogenation unit 910, at least a portion of the olefins in the first hydrocarbon stream may be hydrogenated to paraffins to produce a second hydrocarbon stream. Reaction conditions in the hydrogenation unit 910, may be the same as previously described for System 900.

At least a portion of the second hydrocarbon stream may enter dehydrogenation unit 180 via second conduit 1120. At least a portion of the paraffins in the second hydrocarbon stream may be dehydrogenated using process conditions as previously described for System 100. At least a portion of the resulting olefinic hydrocarbon stream and at least a portion of the unreacted hydrocarbons in the second hydrocarbon stream may form a third hydrocarbon stream.

At least a portion of the third hydrocarbon steam may exit the dehydrogenation unit and enter dimerization unit 710 via third conduit 1130. In dimerization unit 710, at least a portion of the olefins in the third hydrocarbon stream may be dimerized. Conditions of the olefin dimerization may be controlled as described for System 700.

In an embodiment, at least a portion of a fourth hydrocarbon stream may exit dimerization unit 710 and enter separation unit 712 via conduit 714 as depicted in FIG. 12B. In separation unit 712 the reaction mixture may be separated into a produced dimerized olefins stream and a paraffins and unreacted olefins stream through fractional distillation. The paraffins and unreacted olefins stream may contain hydrocarbons with a carbon number less than about 8. At least a portion of the paraffins and unreacted olefins stream may be introduced into dehydrogenation unit 180 via conduit 716; In other embodiments, the paraffins and unreacted olefins stream may be combined with a process stream upstream of the dehydrogenation unit. The produced dimerized olefins stream may exit separation unit 712 and be introduced into second conduit 1120 via conduit 718.

Referring back to FIG. 12A, the fourth hydrocarbon stream may exit dimerization unit 710 and be introduced into alkylation unit 140 via fourth conduit 1140. The fourth hydrocarbon stream includes branched olefins. At least a portion of the olefins in the fourth hydrocarbon stream may alkylate at least a portion of aromatic hydrocarbons using process conditions as previously described for System 100.

In certain embodiments, it may be desirable to adjust the olefin and paraffin concentration in alkylation unit 140 depending on the source of the olefin stream as previously described for System 800. A paraffinic stream may be added to a process stream that contains greater than about 50 percent mono-olefins upstream of an alkylation unit to produce a process stream that is less than about 50 percent mono-olefins. In some embodiments, a fifth hydrocarbon stream containing about 20 percent linear olefins and 80 percent paraffins may be added to a process stream containing primarily branched olefins upstream of an alkylation unit. Alkylation of aromatic hydrocarbons with the combined stream may result in a mixed stream containing branched and linear alkyl aromatic hydrocarbons.

In an embodiment, a fifth hydrocarbon stream may be added to process stream upstream of the alkylation unit that contains an olefin content of between about 1 percent and about 99 percent relative to the total hydrocarbon content. In certain embodiments, an olefin content of the fifth hydrocarbon stream may be between about 5 percent and about 15 percent relative to the total hydrocarbon content. In other embodiments, an olefin content of the fifth hydrocarbon stream may be greater than 80 percent relative to the total hydrocarbon content.

In an embodiment, an olefin content of a fifth hydrocarbon stream ranges between about 1 percent and about 99 percent relative to the total hydrocarbon content. In certain embodiments, an olefin content of a fifth hydrocarbon stream may be between about 5 percent and about 15 percent relative to the total hydrocarbon content. In other embodiments, an olefin content of a fifth hydrocarbon stream may be greater than 80 percent relative to the total hydrocarbon stream.

The alkylation reaction mixture stream may enter separator 160 via fifth conduit 1150. Separation of at least a portion of paraffins and at least a portion of olefins from the alkylation reaction mixture may be accomplished as previously described for System 100. At least two streams may be produced, an unreacted hydrocarbons stream and an alkyl aromatic hydrocarbons product stream. At least a portion of the unreacted hydrocarbons stream may be separated into an aromatic hydrocarbons stream and a paraffins and unreacted olefins stream using generally known techniques (e.g., distillation, solid/liquid separation, absorption, solvent extraction). At least a portion of the separated aromatic hydrocarbons stream may exit separator 160 and be recycled to alkylation unit 140 via sixth conduit 1160. The alkyl aromatic hydrocarbons product stream may be transferred through seventh conduit 1170 to be stored on site, sold commercially, transported off-site, and/or utilized in other processing units (e.g., sulfonation unit).

At least a portion of the paraffins and unreacted olefins stream may exit separator 160 and be combined with other process streams, sent to other processing units and/or be stored on site via eighth conduit 1180. In certain embodiments, the paraffins and unreacted olefins stream may be further separated into a hydrocarbons stream including paraffins and unreacted olefins with a carbon number less than 8. The hydrocarbon stream including paraffins and unreacted olefins with a carbon number less than 8 may be introduced upstream of and/or into dehydrogenation unit 180.

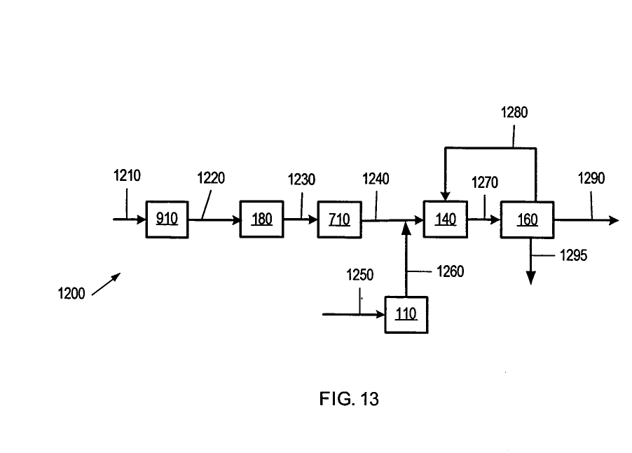

In certain embodiments, a hydrocarbon stream from a dimerization unit may be combined with a hydrocarbon stream from an isomerization unit to produce a combined stream. The combined stream may be introduced into an alkylation unit. Combining streams from the two units may result in a more economically valuable process to produce alkyl aromatic hydrocarbons. Referring to System 1200 as depicted in FIG. 13, a first hydrocarbon stream may be introduced into hydrogenation unit 910 via first conduit 1210. The first hydrocarbon stream includes olefins and paraffins. In hydrogenation unit 910, at least a portion of the olefins in the first hydrocarbon stream may be hydrogenated paraffins to produce a second hydrocarbon stream. Reaction conditions in the hydrogenation unit 910, may be the same as previously described for System 900.

At least a portion of the second hydrocarbon stream may enter dehydrogenation unit 180 via second conduit 1220. At least a portion of the paraffins in the second hydrocarbon stream may be dehydrogenated using process conditions as previously described for System 100. At least a portion of the resulting olefinic hydrocarbon stream and at least a portion of the unreacted hydrocarbons in the second hydrocarbon stream may form a third hydrocarbon stream.

At least a portion of the third hydrocarbon steam may exit the dehydrogenation unit and enter dimerization unit 710 via third conduit 1230. In dimerization unit 710, at least a portion of the olefins may be dimerized. At least a portion of the dimerized olefins exit dimerization unit 710 as a fourth hydrocarbon stream. Conditions of the olefin isomerization may be controlled, as previously described for System 700.

At least a portion of the fourth hydrocarbon stream may exit dimerization unit 710 and enter alkylation unit 140 via fourth conduit 1240. In certain embodiments, the fourth hydrocarbon stream may include olefins with an average carbon number that ranges between 8 and 16. Referring to System 1200 in FIG. 13, at least a portion of a sixth hydrocarbon stream may be introduced into fourth conduit 1240 via sixth conduit 1260 upstream of alkylation unit 140 to produce a combined stream. The sixth hydrocarbon stream may be a stream exiting from isomerization unit 110. Isomerization unit 110 may be fed by a fifth hydrocarbon stream containing olefins and paraffins via a fifth conduit 1250. In isomerization unit 110, at least a portion of the olefins in the fifth hydrocarbon stream may be isomerized to branched olefins to produce the sixth hydrocarbon stream using process conditions previously described for System 100. The sixth hydrocarbon stream may exit isomerization unit 110 via sixth conduit 1260 and be combined with fourth hydrocarbon stream in conduit 1240, as depicted in FIG. 13.

The combined stream may enter alkylation unit 140 and at least a portion of the olefins in the combined stream may alkylate aromatic hydrocarbons to produce alkyl aromatic hydrocarbons. In certain embodiments, the combined hydrocarbon stream may include hydrocarbons with an average carbon number ranging from 7 to 16. Alkylation reaction conditions may be the same as previously described for System 100. The resulting alkylation product may be branched alkyl aromatic hydrocarbons.

A paraffinic stream may be added to a process stream that contains greater than about 50 percent mono-olefins upstream of the isomerization unit to produce a process stream that is less than about 50 percent mono-olefins. In some embodiments, a process stream containing about 20 percent linear olefins and 80 percent paraffins may be added to a process stream containing primarily branched olefins upstream of an isomerization unit.

In certain embodiments, it may be desirable to adjust the olefin and paraffin concentration in the alkylation unit depending on the source of the olefin stream as previously described for System 800 in FIG. 9. In other embodiments, a process stream may be introduced upstream of and/or into the alkylation unit. Alkylation of aromatic hydrocarbons with the combined stream may result in a mixed stream containing branched and linear alkyl aromatic hydrocarbons.

The alkylation reaction mixture stream may enter separator 160 via seventh conduit 1270. Separation of at least a portion of paraffins and at least a portion of olefins from the alkylation reaction mixture may be accomplished as previously described for System 100. At least two streams may be produced, an unreacted hydrocarbons stream and an alkyl aromatic hydrocarbon product stream. At least a portion of the unreacted hydrocarbons stream may be separated into an aromatic hydrocarbons stream and a paraffins and unreacted olefins stream using generally known techniques (e.g., distillation, solid/liquid separation, absorption, solvent extraction). At least a portion of the separated aromatic hydrocarbons stream may exit separator 160 and be recycled to alkylation unit 140 via eighth conduit 1280. The alkyl aromatic hydrocarbon product stream may be transferred through ninth conduit 1290 to be stored on site, sold commercially, transported off-site, and/or utilized in other processing units (e.g., sulfonation unit).

In certain embodiments, the paraffins and unreacted olefins stream may be introduced upstream of and/or into dehydrogenation unit 180 via other ports and/or conduits via tenth conduit 1295. In other embodiments, the paraffins and unreacted olefins stream may be further separated into a hydrocarbons stream including paraffins and unreacted olefins with a carbon number less than 8. At least a portion of the paraffins and unreacted olefins stream maybe introduced upstream of dehydrogenation unit 180 via other ports and/or conduits.

The alkyl aromatic hydrocarbons product stream may be sulfonated in a sulfonation unit to form alkyl aromatic sulfonates. In certain embodiments, alkyl aromatic hydrocarbons may contain branched alkyl groups. Sulfonation of at least a portion of the aromatic hydrocarbons in the alkyl aromatic hydrocarbons product stream may be performed by any method of sulfonation generally known. Examples of sulfonation methods include sulfonation using sulfuric acid, chlorosulfonic acid, oleum or sulfur trioxide. A sulfonation method involving an air/sulfur trioxide mixture is described in U.S. Pat. No. 3,427,342 to Brooks et al., entitled “Continuous Sulfonation Process,” which is incorporated by reference herein. In an embodiment, a sulfur trioxide to alkyl aromatic product molar ratio used for sulfonation is 1.03.

After the sulfonation reaction is complete, the sulfonation reaction mixture may be aged for about thirty minutes and then hydrolyzed with approximately 1% water. The resulting acid mixture may be neutralized with a base to produce a salt of the branched alkyl aromatic hydrocarbon sulfonate. Suitable neutralization bases may be hydroxides of alkali metals and alkaline earth metals, and ammonium hydroxides, which provide the cation of the sulfonate salt.

A general class of branched alkyl aromatic hydrocarbon sulfonates may be characterized by the chemical formula (R-A′-SO₃)ₙM. R represents a radical derived from the branched olefins, having an average carbon number in the range from 4 to 16. In an embodiment, an average carbon number ranges from 7 to 16. In another embodiment, an average carbon number ranges from 10 to 13. A′ may represent a divalent aromatic hydrocarbyl radical, (e.g. a phenyl radical). M may include an alkali metal ion, an alkaline earth metal ion, an ammonium ion, and/or mixtures thereof and n may be a number depending on the valence of the cation(s) M, such that the total electrical charge of the complex is zero. In an embodiment, M may be sodium, magnesium or potassium ions. Magnesium and potassium ions may promote water solubility and performance of the alkyl aromatic hydrocarbon sulfonate. Examples of ammonium ions may include, but are not limited to, monoethanol amine, diethanol amine and triethanol amine. In certain embodiments, an ammonium ion may be represented by NH₄⁺. Alkyl aromatic hydrocarbon sulfonate salts may be stored and/or sold as a solution (e.g. 30% aqueous solution), slurry (e.g., 60% aqueous slurry) and/or flakes (e.g., 90% dried flakes).

Branched alkyl aromatic sulfonates may be used in a wide variety of applications. An example of an application includes detergent formulations. Detergent formulations include, but are not limited to, granular laundry detergent formulation, liquid laundry detergent formulations, liquid dishwashing detergent formulations and miscellaneous formulations. Examples of miscellaneous formulations include, but are not limited to, general purpose cleaning agents, liquid soaps, shampoos and liquid scouring agents.

Granular laundry detergent formulations may include a number of components besides the alkyl aromatic hydrocarbon sulfonate surfactant (e.g., other surfactants, builders, co-builders, bleaching agents, bleaching agent activators, foam controlling agents, enzymes, anti-graying agents, optical brighteners and stabilizers). Examples of other surfactants include, but are not limited to, ionic, nonionic, amphoteric or cationic surfactants.

Liquid laundry detergent formulations may include the same components as granular laundry detergent formulations. In certain embodiments, liquid laundry detergent formulations may include less of an inorganic builder component than granular laundry detergent formulations. Hydrotropes may be present in the liquid detergent formulations. General purpose cleaning agents may include other surfactants, builders, foam control agents, hydrotropes and solubilizer alcohols.

Formulations may include a large amount of the builder and co-builder components. In some embodiments, builder and co-builder components may be about 90 percent by weight. To intensify the cleaning action, the builder and co-builder components may, in other embodiments, be in amounts from about 5 percent to about 35 percent by weight, based on the weight of the formulation. Examples of common inorganic builders include, but are not limited to, phosphates, polyphosphates, alkali metal carbonates, silicates and sulfonates. Examples of organic builders include, but are not limited to, polycarboxylates, aminocarboxylates such as ethylenediaminetetraacetates, nitrilotriacetates, hydroxycarboxylates, citrates, succinates, and substituted and unsubstituted alkane di- and polycarboxylic acids. Another type of builder, useful in granular laundry and built liquid laundry agents, includes various substantially water-insoluble materials, which are capable of reducing the water hardness. An example of process to reduce water hardness is an ion exchange process. In an embodiment, complex sodium aluminosilicates, known as type A zeolites, may be useful for this purpose.

Formulations may include percompounds with a bleaching action. Examples of percompounds include, but are not limited to, perborates, percarbonates, persulfates and organic peroxy acids. Formulations containing percompounds may also include stabilizing agents. Examples of stabilizing agents include, but are not limited to, magnesium silicate, sodium ethylenediaminetetraacetate or sodium salts of phosphonic acids. In some embodiments, bleach activators may be used to increase the efficiency of the inorganic persalts at lower washing temperatures. Substituted carboxylic acid amides, tetracetylethylenediamine, substituted carboxylic acids may be useful for lower washing temperatures in other embodiments. Examples of substitute carboxylic acids include, but are not limited to, isononyloxybenzenesulfonate and sodium cyanamide.

Examples of suitable hydrotropic substance include, but are not limited to, alkali metal salts of benzene, toluene and xylenes, sulfonic acids; alkali metal salts of formic acid; citric and succinic acid; alkali metal chlorides; urea; and mono-, di- and tri-ethanolamine. Examples of solubilizer alcohols include, but are not limited to, ethanol, isopropanol, mono- or polyethylene glycols, monopropylene glycol and ether alcohols.

Examples of foam control agents include, but are not limited to, high molecular weight fatty acid soaps, paraffinic hydrocarbons and silicon containing defoamers. In an embodiment, hydrophobic silica particles are efficient foam control agents in laundry detergent formulations.

Examples of known enzymes that are effective in the laundry detergent formulations include, but are not limited to protease, amylase and lipase. Enzymes, which have an optimum performance at the design conditions of the washing and cleaning agent, may be used.

A large number of fluorescent whiteners are described in the literature. For the laundry washing formulations, the derivatives of diaminostilbene, disulfonates and substituted distyrybiphenyl maybe used.

Water-soluble colloids of an organic nature may be used as anti-graying agents. Examples of water-soluble anti-graying agents include, but are not limited to, polyanionic polymers such as polymers and copolymers of acrylic and maleic acid, cellulose derivatives such as carboxymethyl cellulose methyl- and hydroxethylcellulose.

Formulations may typically include one or more inert components. For example, the balance of liquid detergent formulations may typically be an inert solvent or diluent, e.g. water. Powdered or granular detergent formulations typically contain quantities of inert filler or carrier materials.

### EXAMPLES

### Example 1

Isomerization of Olefins in a Fischer-Tropsch derived Hydrocarbon Stream:

Carbon monoxide and hydrogen were reacted under Fischer-Tropsch process conditions to yield a hydrocarbon mixture of linear paraffins, linear olefins, a minor amount of dienes and a minor amount of oxygenates. The Fischer-Tropsch hydrocarbon stream was separated into different hydrocarbon streams using fractional distillation techniques. A hydrocarbon stream containing olefins and paraffins with an average number of carbon atoms between 8 and 10 was obtained. The composition of the resulting C₈-C₁₀ hydrocarbon stream was analysed by gas chromatography and is tabulated in Table 1.

TABLE 1

| Fischer-Tropsch Hydrocarbon | |
|---|---|
| Stream Composition | Wt. % |
| C₇ and lighter hydrocarbons | 0.12 |
| C₈ branched olefins | 0.02 |
| C₈ linear olefins | 0.75 |
| 1-Octene | 0.69 |
| n-Octane | 2.21 |
| C₉ branched olefins | 0.16 |
| C₉ linear olefins | 8.52 |
| 1-Nonene | 8.07 |
| n-Nonane | 20.03 |
| C₁₀ branched olefins | 0.28 |
| C₁₀ linear olefins | 22.92 |
| 1-Decene | 20.87 |
| n-Decane | 41.12 |
| C₁₁ and heavier hydrocarbons | 0.21 |
| C₉-C₁₁ alcohols | 3.56 |

A zeolite catalyst used for isomerization of linear olefins in the hydrocarbon stream was prepared in the following manner. Ammonium-ferrierite (645 grams) exhibiting a 5.4% loss on ignition and exhibiting the following properties: molar silica to alumina ratio of 62:1, surface area of 369 square meters per gram (P/Po=0.03), soda content of 480 ppm and n-hexane sorption capacity of 7.3 g per 100 g of ammonium-ferrierite was loaded into a Lancaster mix muller. CATAPAL® D alumina (91 grams) exhibiting a loss on ignition of 25.7% was added to the muller. During a five-minute mulling period, 152 milliliters of deionized water was added to the alumina/ammonium-ferrierite mixture. Next, a mixture of 6.8 grams glacial acetic acid, 7.0 grams of citric acid and 152 milliliters of deionized water was slowly added to the alumina/ammonium-ferrierite mixture in the muller to peptize the alumina. The resulting alumina/ammonium-ferrierite/acid mixture was mulled for 10 minutes. Over a period of 15 minutes, a mixture of 0.20 grams of tetraamine palladium nitrate in 153 grams of deionized water was slowly added to mulled alumina/ammonium-ferrierite/acid mixture. The resulting mixture exhibited a 90:10 ratio of zeolite to alumina and a loss on ignition of 43.5%. The zeolite/alumina mixture was shaped by extruding the mixture through a stainless steel die plate (1/16″ holes) of a 2.25 inch Bonnot extruder.

The moist zeolite/alumina extrudate was dried at 125° C. for 16 hours. After drying, the zeolite/alumina extrudate was longsbroken manually. The zeolite/alumina extrudate was calcined in flowing air at 200° C. for two hours. The temperature was raised to a maximum temperature of 500° C. and the zeolite/alumina extrudate was calcined for an additional two hours to yield an isomerization catalyst. The isomerization catalyst was allowed to cool in a dessicator under a nitrogen atmosphere.

Stainless steel tubing, 1 inch OD, 0.6 inch ID and 26 inches long, was used as an isomerization reactor. A thermowell extended 20 inches from the top of the stainless steel reactor tube. To load the reactor tube, the reactor tube was inverted and a piece of glass wool was transferred down the wall of the reactor tube, over the thermowell and positioned at the bottom of the reactor tube to serve as a plug for the reactor tube. Silicon carbide (20 mesh) was added to a depth of about 6 inches to the reactor tube. A second piece of glass wool was placed over the silicon carbide. A mixture of 6.0 grams of the isomerization catalyst particles (6-20 mesh) and 45 grams of fresh silicon carbide (60-80 mesh) was added to the reactor tube in two parts. The two-part addition distributed the isomerization catalyst evenly in the reactor tube and resulted in an isomerization catalyst bed of about 10 inches in length. A third piece of glass wool was added to the top of the catalyst in the reactor tube. Silicon carbide (20 mesh) was layered onto the third piece of glass wool. A fourth piece of glass wool was positioned over the silicon carbide to serve as a plug for the bottom of the reactor tube. To monitor the temperature of the reaction at various points in the reactor tube, a multipoint thermocouple was inserted into the thermowell of the reactor tube. The temperature above, below and at three different places in the catalyst bed was monitored. The reactor tube was inverted and installed in the furnace. The reactor tube was heated to the operating temperature of 280° C. over a four-hour period under flowing nitrogen. Once the temperature of 280° C. was obtained, the reactor tube was held at the operating temperature for an additional two hours to condition the isomerization catalyst.

After conditioning the isomerization catalyst, the hydrocarbon stream was pumped through the reactor tube at a flow rate of 60 g/hr. Nitrogen, at a flow rate of 6 L/hr, was passed over the isomerization catalyst simultaneously with the hydrocarbon stream. The hydrocarbon stream was vaporized before contacting the isomerization catalyst. The reactor tube was operated at an outlet pressure of 20 kPa above atmospheric pressure.

In Table 2, the weight percent of C₈-C₁₀ branched olefins, C₈-C₁₀ linear olefins and C₈-C₁₀ paraffins in the hydrocarbon stream at 0 hours and in the reactor tube effluent after 24 and 48 hours of isomerization is tabulated. Greater than 90% of the linear olefins in the hydrocarbon stream were converted into branched olefins in the isomerization reactor. During the isomerization step, a small amount of material boiling below C₈ was generated from cracking side reactions. In addition, a portion of the C₉-C₁₁ alcohols present in the feed was dehydrated to yield additional olefins in the product. The average number of alkyl branches on the C₈-C₁₀ olefins in the product was found to be 1.0 as determined by the ¹H NMR analylsis.

TABLE 2

| Fischer-Tropsch Hydrocarbon | | | |
|---|---|---|---|
| Stream Composition During | 0 Hr | 24 Hr | 48 Hr |
| Isomerization Reaction | Wt. % | Wt. % | Wt. % |
| C₈-C₁₀ branched olefins | 0.46 | 33.04 | 33.16 |
| C₈-C₁₀ linear olefins | 32.19 | 2.52 | 2.54 |
| C₈-C₁₀ paraffins | 63.19 | 63.32 | 63.27 |
| Branched to linear C₈-₁₀ olefin ratio | 0.1 | 13.1 | 13.1 |

### Example 2

Isomerization of 1-Dodecene: 1-Dodecene was obtained from Shell Chemical Co. The composition of 1-dodecene, as assayed by gas chromatography, is tabulated in Table 3.

1-Dodecene was isomerized using the same reactor tube design and isomerization catalyst preparation as described in Example 1. A stream of 1-dodecene was pumped through a reactor tube at a flow rate of 90 g/hr. Nitrogen, at a flow rate of 6 L/hr, was passed over the isomerization catalyst simultaneously with the stream of 1-dodecene. The stream of 1-dodecene was vaporised before contacting the isomerization catalyst. The reactor tube was operated at an outlet pressure of 20 kPa above atmospheric pressure and a temperature of 290° C.

Table 4 is a tabulation of the weight percent of less than C₁₀, C₁₀-C₁₄ and greater than C₁₄ molecules in 1-dodecene at 0 hours and the reactor tube effluent after 168 and 849 hours. Linear C₁₀-C₁₄ olefins were converted in a 94% yield to branched C₁₀-C₁₄ olefins after a 168 hr processing time. During the isomerization step, less than 3 weight percent of material boiling below C₁₀ was generated from cracking side reactions. The average number of alkyl branches on the C₁₀-C₁₄ olefins in the product was determined to be 13 by ¹H NMR analysis.

TABLE 3

| 1-Dodecene Composition | Wt. % |
|---|---|
| 1-Dodecene | 98.0 |
| Other C₁₀-C₁₄ Olefins | 1.2 |
| <C₁₀ hydrocarbons | 0.2 |
| >C₁₄ hydrocarbons | 0.2 |
| Paraffins | 0.4 |
| Total C₁₀-C₁₄ Hydrocarbons | 99.6 |

TABLE 4

| 1-Dodecene Stream Composition | 0 Hr, | 168 Hr. | 849 Hr. |
|---|---|---|---|
| During Isomerization Reaction | wt % | wt % | wt % |
| <C₁₀ hydrocarbons | 0.2 | 2.5 | 2.4 |
| C₁₀-C₁₄ hydrocarbons | 99.6 | 97.2 | 97.4 |
| >C₁₄ hydrocarbons | 0.2 | 0.3 | 0.2 |
| Branched C₁₀-C₁₄ Olefins | 0.6 | 93.2 | 93.4 |
| Linear C₁₀-C₁₄ Olefins | 99.0 | 2.8 | 2.9 |
| Paraffins | 1.0 | 2.0 | 1.9 |

### EXAMPLE 3

DEHYDROGENATION OF DODECANE WITH MINIMAL ISOMERIZATION: Dodecane was obtained from Aldrich Chemical Company and stored under nitrogen before being processed. The composition of dodecane, as assayed by gas chromatography, is tabulated in Table 5.

TABLE 5

| Dodecane Composition | Wt. % |
|---|---|
| Dodecane | 99.3 |
| <C₁₀ hydrocarbons | <0.1 |
| C₁₀, C₁₁, C₁₃ and C₁₄ hydrocarbons | <0.6 |
| >C₁₄ hydrocarbons | <0.1 |
| Other C₁₀-C₁₄ olefins | <0.1 |

A paraffin dehydrogenation catalyst was prepared according to Example 1 (catalyst A) of U.S. Pat. No. 4,430,517 to Imai et al., entitled “Dehydrogenation Process Using A Catalytic Composition”, which is incorporated by reference herein. The resulting catalyst included 0.8 wt. % platinum, 0.5 wt. % tin, 2.7 wt. % potassium and 1.3 wt. % chlorine on a gamma-alumina support. The atomic ratio of potassium to platinum for this catalyst was 16.8.

The dehydrogenation catalyst was prepared by dissolving substantially pure aluminum pellets in a hydrochloric acid solution. An amount of stannic chloride was added to the resulting solution to provide a final composite containing 0.5 weight % tin and stirred to distribute the tin component evenly throughout the mixture. Hexamethylenetetramine was added to the resulting tin mixture and the resulting tin-amine mixture was dropped into an oil bath in a manner to form spherical particles having an average particle diameter of about 1/16 inch. The spheres were aged, washed with an ammoniacal solution, dried and calcined to form a spherical gamma-alumina carrier material. The resulting spheres contained about 0.5 weight % tin in the form of tin oxide. More details about the method of preparing the alumina carrier material are disclosed in U.S. Pat. No. 2,620,314 to Hoesktra, entitled “Spheroidal Alumina,” which is incorporated by reference herein.

The tin-alumina composite was contacted with a deionized solution of chloroplatinic acid and hydrochloric acid (2 weight percent based on alumina weight) in a rotary drier for 15 minutes at room temperature. The amount of chloroplatinic acid used was the amount necessary to incorporate 0.8 weight percent platinum into the tin-alumina composite. The solution was then heated and purged with nitrogen to remove water resulting in a platinum-chlorine-tin-alumina composite. The incorporated chlorine was removed by heating the platinum-chlorine-tin-alumina composite to 550° C. and treating the composite with a 50/50 air/80° C. steam mixture at a gas hourly space velocity (GHSV) of 300 hr⁻¹. After treatment with the air/steam mixture, the platinum-tin-alumina composite contained less than 0.1 weight percent chlorine.

The platinum-tin-alumina composite was contacted with a deionized water solution of potassium nitrate. The amount of potassium nitrate used was the amount necessary to incorporate 2.7 weight percent of potassium in the platinum-tin-alumina composite. The water was removed from the platinum-tin-potassium alumina composite by heating the composite to 100° C. under a purge of dry air (1000 hr⁻¹ GHSV) for 0.5 hour. The temperature was raised to 525° C. and the platinum-tin-potassium alumina composite was treated with a stream of hydrochloric acid (12 cc/hr, 0.9 M HCl) and a stream of 50/50 air/80° C. steam mixture (300 hr⁻¹ GHSV) to incorporate chlorine into the platinum-tin-potassium-alumina composite. The platinum-tin-potassium-chlorine-alumina composite was dried at 525° C. under a purge of dry air (1000 hr⁻¹ GHSV). The resulting catalyst spheres had an average particle diameter of 1/16 inch and were crushed and sized into 6-20 mesh particle before testing.

Stainless steel tubing, 1 inch OD, 0.6 inch ID and 26 inches long, was used as an isomerization reactor. A thermowell extended 20 inches from the top of the stainless steel reactor tube. To load the reactor tube, the reactor tube was inverted and a piece of glass wool was transferred down the wall of the reactor tube, over the thermowell and positioned at the bottom of the reactor tube to serve as a plug for the reactor tube. Silicon carbide (20 mesh) was added to a depth of about 6 inches to the reactor tube. A second piece of glass wool was placed over the silicon carbide. A mixture of 6.0 grams of platinum-tin on alumina catalyst particles (6-20 mesh) and 45 grams of fresh silicon carbide (60-80 mesh) was added to the reactor tube in two parts. The two-part addition distributed the catalyst evenly in the reactor tube and resulted in a catalyst bed of about 10 inches in length. A third piece of glass wool was added to the top of the catalyst in the reactor tube. Silicon carbide (20 mesh) was layered onto the third piece of glass wool. A fourth piece of glass wool was positioned over the silicon carbide to serve as a plug for the bottom of the reactor tube. To monitor the temperature of the reaction at various points in the reactor tube, a multipoint thermocouple was inserted into the thermowell of the reactor tube. The temperature above, below and at three different places in the catalyst bed was monitored. The reactor tube was inverted and installed in the furnace. The reactor tube was purged with nitrogen. The reactor tube was heated to the operating temperature of 425° C. over a four-hour period under flowing nitrogen (250 standard liters per hour). Once the temperature of 425° C. was obtained, the reactor tube was held at the operating temperature for an additional two hours. The catalyst was presulfided by flowing a 1% mixture of hydrogen sulfide gas in hydrogen gas at 425° C. for five minutes through the reactor tube. After 5 minutes, the hydrogen sulfide in hydrogen gas flow was switched to a hydrogen gas flow through the reactor tube.

After presulfiding the catalyst, the reactor tube was maintained at 425° C. for eight hours. After eight hours, the reactor tube pressure was increase to 25 psig with hydrogen gas. Dodecane was pumped through the reactor tube at a flow rate of 40 g/hr at a hydrogen flow rate of 125 standard liters per hour. After four hours, the dodecane stream was increased to 80 g/hr. After obtaining a flow rate of 80 g/hr, the reactor tube temperature was raised to 460° C. The reactor tube was sampled every eight hours after obtaining the operating temperature of 460° C.

After twenty-four hours the weight percent of dodecane was 11.4 weight percent as depicted in Table 6. At a temperature of 479° C., the conversion of dodecane to olefins was 16 weight percent after twenty-four hours. Of the olefins, formed 84 weight percent were mono olefins, 4.1 weight percent were aromatic compounds and 7.5 weight percent were di-olefins. Of the total amount of olefins formed, 6 percent were branched, as determined by ¹H NMR analysis.

TABLE 6

| Test Results. | Example 3 |
|---|---|
| Conversion (wt. %) after 24 | 11.4 |
| hours on-stream at 460° C. | |
| Temperature required for 16 | 479° C. |
| wt. % conversion | |
| Selectivity to mono olefins | 84 wt. % |
| at 16 wt. % conversion. | |
| Selectivity to aromatics at | 4.1 wt. % |
| 16 wt. % conversion. | |
| Selectivity to di-olefins at | 7.5 wt. % |
| 16 wt. % conversion. | |
| % Branched C₁₂ olefins in | 6 |
| total C₁₂ olefins, (wt. %) | |

### EXAMPLE 4

DEHYDROGENATION—ISOMERIZATION OF DODECANE: Dodecane was obtained from Aldrich Chemical Company and stored under nitrogen before being processed. The composition of dodecane, as assayed by gas chromatography, is tabulated in Table 5.

A dehydrogenation-isomerization catalyst was prepared in the following manner. Ammonium-ferrierite (645 grams) exhibiting a 5.4% loss on ignition and exhibiting the following properties: molar silica to alumina ratio of 62:1, surface area of 369 square meters per gram (P/Po=0.03), soda content of 480 ppm and n-hexane sorption capacity of 7.3 g per 100 g of ammonium-ferrierite was loaded into a Lancaster mix muller. CATAPAL® D alumina (91 grams) exhibiting a loss on ignition of 25.7% was added to the muller. During a five-minute mulling period, 152 milliliters of deionized water was added to the alumina/ammonium-ferrierite mixture. Next, a mixture of 6.8 grams glacial acetic acid, 7.0 grams of citric acid and 152 milliliters of deionized water was slowly added to the alumina/ammonium-ferrierite mixture in the muller to peptize the alumina. The resulting alumina/ammonium-ferrierite/acid mixture was mulled for 10 minutes. Over a period of 15 minutes, a mixture of 0.20 grams of tetraamine palladium nitrate in 153 grams of deionized water was slowly added to mulled alumina/ammonium-ferrierite/acid mixture. The resulting mixture exhibited a 90:10 ratio of zeolite to alumina and a loss on ignition of 43.5%. The zeolite/alumina mixture was shaped by extruding the mixture through a stainless steel die plate (1/16″ holes) of a 2.25 inch Bonnot extruder.

Six grams of the resulting zeolite/alumina mixture was impregnated with an aqueous solution of chloroplatinic acid to incorporate 0.8 wt. % platinum into the 1/16 inch extrudate. The moist zeolite/alumina platinum impregnated extrudate was dried at 125° C. for 2 hours in flowing air. The temperature was raised to a maximum temperature of 500° C. and the zeolite/alumina platinum impregnated extrudate was calcined to yield a dehydrogenation-isomerization catalyst. The calcined catalyst was crushed and sized into 6-20 mesh particles before testing.

Dodecane was dehydrogenated and isomerized using the same reactor tube design as described in Example 3. A 16.1 weight percent conversion of dodecane to olefins was observed after twenty-fours-hours at 459° C. As tabulated in Table 7, of the olefins formed 86 weight percent were mono olefins, 1.2 weight percent were aromatic compounds and 6.8 weight percent were di-olefins. Of the total amount of olefins formed, 86 percent were branched, as determined by the ¹H NMR analysis.

TABLE 7

| Test Results. | Example 4 |
|---|---|
| Conversion (wt. %) After 24 | 16.1 |
| hours on-stream at 460° C. | |
| Temperature required for 16 | 459° C. |
| wt. % conversion | |
| Selectivity to mono olefins | 86 wt. % |
| at 16 wt. % conversion. | |
| Selectivity to aromatics at | 1.2 wt. % |
| 16 wt. % conversion. | |
| Selectivity to di-olefins at | 6.8 wt. % |
| 16 wt. % conversion. | |
| % Branched C₁₂ olefins in | 86 |
| total C₁₂ olefins, (wt. %) | |

### Example 5

DEHYDROGENATION—ISOMERIZATION CATALYST. A zeolite portion of a dehydrogenation-isomerization catalyst was prepared as described in Example 4. Six grams of the resulting zeolite/alumina mixture was impregnated with an aqueous solution of 5′ tetraamine palladium nitrate to incorporate 0.8 wt. % palladium into the 1/16 inch extrudates.

The moist zeolite/alumina palladium impregnated extrudate was dried at 125° C. for 2 hours in flowing air. The temperature was raised to a maximum temperature of 500° C. and the zeolite/alumina platinum impregnated extrudate was calcined to yield a dehydrogenation-isomerization catalyst. The calcined catalyst was carefully crushed and sized into 6-20 mesh particles before testing.

### EXAMPLE 6

DEHYDROGENATION—ISOMERIZATION CATALYST: A dehydrogenation-isomerization catalyst was prepared by the methods described for catalyst D of U.S. Pat. No. 5,648,585 to Murray et al., entitled “Process For Isomerizing Linear Olefins To Isoolefins”, which is incorporated by reference herein.

Ammonium-ferrierite having a molar silica to alumina ratio of 62:1, a surface area of 369 m2/g (P/Po=0.03), a soda content of 480 ppm wt and a n-hexane sorption capacity of 7.3 grams per 100 grams of zeolite was used. The catalyst components were mulled using a Lancaster mix muller. The mulled catalyst material was extruded using a Bonnot pin barrel extruder. The binder utilized was CATAPAL® D alumina from Sasol. METHOCEL® F4M, hydroxypropyl methylcellulose, from The Dow Chemical Company was used as an extrusion aid.

The Lancaster mix muller was loaded with 632 grams of ammonium ferrierite (LOI of 3.4%) and 92 grams of CATAPAL®D alumina (LOI of 26.2%). The alumina was blended with the ferrierite for five minutes during which time 156 milliliters of de-ionized water was added. A mixture of 6.8 grams of glacial acetic acid and 156 milliliters of de-ionized water were added slowly to the muller in order to peptize the alumina. The mixture was mix-mulled for 10 minutes. Tetraamine platinum nitrate and Tetraamine palladium nitrate were added to the mix-muller in order to produce a catalyst that contained 0.25 wt. % palladium and 0.55 wt. % platinum. Ten grams of METHOCEL® F4M hydroxypropyl methylcellulose was added and the zeolite/alumina was mulled for 15 additional minutes. The extrudate was transferred to a Bonnot pin barrel extruder and extruded using a stainless steel die plate with 1/16 inch holes. The extrudate was dried at 120° C. for 16 hours and then calcined in air at 500° C. for 2 hours. The calcined catalyst was carefully crushed and sized into 6-20 mesh particles before testing.

### EXAMPLE 7

DIMERIZATION OF 1-HEXENE: A dimerization catalyst for the dimerization of a C₆ olefin stream was prepared by the method described for Example 1 in U.S. Pat. No. 5,169,824 to Saleh et al., entitled “Catalyst Comprising Amorphous NiO On Silica/Alumina Support”, which is incorporated by reference herein.

An aluminosilicate cogel (100 gram, 87% by weight SiO₂-13% by weight Al₂O₃) was dispersed in distilled water (2000 mL). Aluminosilicate cogel may be obtained from Ineos Silicas, Netherlands BV, as Synclist-13. Nitric acid was added to the aluminosilicate/water dispersion with stirring until a pH of 2.7 was obtained. The resulting acidic mixture was filtered and the aluminosilicate solid washed with distilled water until the filtrate exhibited a pH of 5.7. The recovered aluminosilicate solid was dispersed distilled water and nitric acid was added until a pH of 2.7 was obtained. The resulting acidic mixture was filtered and the resulting aluminosilicate solid was washed with distilled water until the filtrate exhibited a pH of 5.7. The recovered aluminosilicate solid was dried for 16 hours at 110° C. in an air atmosphere and thereafter calcined at 500° C. for 16 hours under an air atmosphere.

Ni(NO₃).2.6.H₂O (67.38 gram) was dissolved in of distilled water (700 mL) and heated to a temperature of 32° C. to result in a solution having a pH of 5.7. The aluminosilicate solid (35 gram) was added over time to the nickel solution resulting in a nickel/aluminosilicate slurry. The pH of the nickel/aluminosilicate slurry was approximately 3.9. The nickel/aluminosilicate slurry was neutralized by adding a solution of NH₄CO₃ (33.69 gram) in distilled water (200 mL) drop wise over 30 minutes until the pH of the slurry was approximately 6.9. The neutral slurry was stirred for 30 minutes at 32° C. and then filtered to obtain a solid. The recovered solid was slurried twice with water to the original volume of the nickel/aluminosilicate slurry, stirred for 5 minutes and then filtered to obtain a solid. The resulting solid was dried at 110° C. for 16 hours in an air atmosphere. Calcination of the solid was performed by heating the solid under an air atmosphere at increasing temperatures. Initially, the solid was heated to 232° C. for 1 hour. The temperature was raised to 371° C. and the solid heated for 2 hours. After 2 hours, the temperature was raised to 592° C. and the solid was heated for 16 hours. The resulting in a NiO catalyst dispersed on an aluminosilicate support was crushed and carefully sized to slightly greater than 60 mesh before testing.

A 15 mL reactor tube of an autoclave unit was charged with the NiO catalyst (0.335 grams), 1-hexene (3.35 grams), and a gas chromatography standard (0.67 grams linear tetradecane). Autoclave units of the type “Endeavour” from Argonaut Technologies, United Kingdom, were used to perform the dimerization experiments. The gas cap of the reactor tube was flushed with nitrogen and the reactor tube was heated to 160° C. Once the reaction temperature of 160° C. was obtained, the reaction mixture was maintained for 10 hours and then cooled to room temperature. The reaction mixture was filtered to remove the NiO catalyst and the filtrate was analysed by gas chromatography. The dimerization results are tabulated in Table 8.

TABLE 8

| Test Results. | Example 7 |
|---|---|
| Conversion (wt. %) | 59 |
| C₁₂ olefin dimer (wt %) | 22 |
| % Conversion of branched C₁₂ | 77 |
| olefins to total C₁₂ olefins | |

### EXAMPLE 8

DIMERIZATION OF DILUTED 1-HEXENE: A 15 mL reactor tube of the autoclave unit was charged with the NiO catalyst (0.335 grams) prepared as described in Example 7,1-hexene (1.675 grams), hexane (1.675 grams), and a gas chromatography standard (0.67 grams linear tetradecane. The gas cap of the reactor tube was flushed with nitrogen and the reactor tube was heated to 160° C. Once the reaction temperature of 160° C. was obtained, the reaction mixture was maintained for eight hours and then cooled to room temperature. The reaction mixture was filtered to remove the NiO catalyst and the filtrate was analyzed by gas chromatography. The dimerization results are tabulated in Table 9.

TABLE 9

| Test Results. | Example 8 |
|---|---|
| Conversion (wt. %) | 54 |
| C₁₂ olefin dimer (wt %) | 8 |
| % Conversion of branched | 82 |
| C₁₂ olefins to total C₁₂ | |
| olefins | |

In this patent, certain U.S. patents, U.S. patent applications and other materials (e.g., articles) have been incorporated by reference. The text of such U.S. patents, U.S. patent applications and other materials is, however, only incorporated by reference to the extent that no conflict exists between such text and the other statements and drawings set forth herein. In the event of such conflict, then any such conflicting text in such incorporated by reference U.S. patents, U.S. patent applications and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method for the production of alkyl aromatic hydrocarbons, comprising:
introducing a first hydrocarbon stream comprising olefins and paraffins into an isomerization unit, wherein the isomerization unit is configured to isomerize at least a portion of linear olefins in the first hydrocarbon stream to branched olefins, and wherein at least a portion of the unreacted components of the first hydrocarbon stream and at least a portion of the produced branched olefins form a second hydrocarbon stream;
introducing at least a portion of the second hydrocarbon stream and aromatic hydrocarbons into an alkylation unit, wherein the alkylation unit is configured to alkylate at least a portion of the aromatic hydrocarbons with at least a portion of the olefins in the second hydrocarbon stream to produce alkyl aromatic hydrocarbons, wherein at least a portion of the produced alkyl aromatic hydrocarbons comprise a branched alkyl group, and wherein at least a portion of the unreacted components of the second hydrocarbon stream, at least a portion of the aromatic hydrocarbons and at least a portion of the produced alkyl aromatic hydrocarbons form an alkylation reaction stream;
separating alkyl aromatic hydrocarbons from the alkylation reaction stream to produce an unreacted hydrocarbons stream and an alkyl aromatic hydrocarbons stream; the unreacted hydrocarbons stream comprising at least a portion of the unreacted components of the second hydrocarbon stream and aromatic hydrocarbons;
separating at least a portion of the paraffins and at least a portion of the olefins from the unreacted hydrocarbons stream to produce an aromatic hydrocarbons stream and a paraffins and unreacted olefins stream; and
introducing at least a portion of the paraffins and unreacted olefins stream into a dehydrogenation unit, wherein the dehydrogenation unit is configured to dehydrogenate at least a portion of paraffins in the paraffins and unreacted olefins stream to produce olefins, and wherein at least a portion of the produced olefins exit the dehydrogenation unit to form an olefinic hydrocarbon stream; and
introducing at least a portion of the olefinic hydrocarbon stream into the isomerization unit.

2. The method of claim 1, wherein the first hydrocarbon stream is produced from an olefin oligomerization process.

3. The method of claim 1, wherein the first hydrocarbon stream is produced from a Fischer-Tropsch process.

4. The method of claim 1, wherein the first hydrocarbon stream comprises olefins and paraffins having a carbon number from 10 to 13.

5. The method of claim 1, wherein the first hydrocarbon stream comprises olefins and paraffins having a carbon number from 10 to 16.

6. The method of claim 1, wherein the isomerization unit is operated at a reaction temperature between about 200° C. and about 500° C.

7. The method of claim 1, wherein the isomerization unit is operated at a reaction pressure between about 0.1 atmosphere and about 10 atmospheres.

8. The method of claim 1, wherein at least a portion of the branched olefins comprise an average number of branches per total olefin molecules of at least 0.7.

9. The method of claim 1, wherein at least a portion of the branched olefins comprise methyl and ethyl branches.

10. The method of claim 1, wherein at least a portion of the branched olefins comprise an average number of branches per total olefin molecules of less than about 2.5.

11. The method of claim 1, wherein at least a portion of the branched olefins comprise an average number of branches per total olefin molecules of between about 0.7 and about 2.0.

12. The method of claim 1, wherein at least a portion of the branched olefins comprise an average number of branches per total olefin molecules of between about 0.7 and about 1.5.

13. The method of claim 1, wherein at least a portion of the branched olefins comprise an average number of branches per total olefin molecules of between about 1.0 and about 1.5.

14. The method of claim 1, wherein greater than about 50 percent of the branched groups on the branched olefins are methyl groups.

15. The method of claim 1, wherein less than about 10 percent of the branched groups on the branched olefins are ethyl groups.

16. The method of claim 1, wherein less than about 5 percent of the branched groups on the branched olefins are groups other than methyl or ethyl groups.

17. The method of claim 1, wherein the branched olefins have less than about 0.5 percent aliphatic quaternary carbon atoms.

18. The method of claim 1, wherein the branched olefins have less than about 0.3 percent aliphatic quaternary carbon atoms.

19. The method of claim 1, wherein the alkylation unit is configured to produce greater than about 50 percent of monoalkylated aromatic hydrocarbons.

20. The method of claim 1, wherein the alkylation unit is configured to produce greater than about 85 percent of monoalkylated aromatic hydrocarbons.

21. The method of claim 1, wherein a molar ratio of the aromatic hydrocarbons to the branched olefins is between about 0.1 and about 2.0 in the alkylation unit.

22. The method of claim 1, wherein the alkylation unit is operated at a reaction temperature between about 30° C. and about 300° C.

23. The method of claim 1, wherein the aromatic hydrocarbons comprise benzene.

24. The method of claim 1, wherein the alkyl aromatic hydrocarbons comprise alkyl benzenes.

25. The method of claim 1, wherein the branched alkyl groups of the alkyl aromatic hydrocarbons comprise 0.5 percent or less aliphatic quaternary carbon atoms, and an average number of branches per alkyl group of at least 0.7, the branches comprising methyl and ethyl branches.

26. The method of claim 1, further comprising adjusting a ratio of olefins to paraffins introduced into the isomerization unit by adding at least a portion of a paraffinic hydrocarbon stream into the isomerization unit.

27. The method of claim 1, further comprising:
adjusting a ratio of olefins to paraffins introduced into the isomerization unit by combining a paraffinic hydrocarbon stream with at least a portion of the first hydrocarbon stream upstream of the isomerization unit to form a combined stream; and
introducing the combined stream into the isomerization unit.

28. The method of claim 1, further comprising adjusting a ratio of olefins to paraffins introduced into the alkylation unit by adding at least a portion of a third hydrocarbon stream into the alkylation unit.

29. The method of claim 1, further comprising adjusting a ratio of olefins to paraffins introduced into the alkylation unit by adding at least a portion of a third hydrocarbon stream into the alkylation unit, wherein the third hydrocarbon stream comprises greater than about 90 percent paraffins by weight.

30. The method of claim 1, further comprising:
adjusting a ration of olefins to paraffins introduced into the alkylation unit by combing at least a portion of a third hydrocarbon stream with at least a portion of the second hydrocarbon stream upstream of the alkylation unit to form a combined stream; and
introducing the combined stream into the alkylation unit.

31. The method of claim 1, further comprising:
adjusting a ratio of olefins to paraffins introduced into the alkylation unit by combining at least a portion of a third hydrocarbon stream with at least a portion of the second hydrocarbon stream upstream of the alkylation unit to form a combined stream, wherein the third hydrocarbon stream comprises greater than about 90 percent paraffins by weight; and
introducing the combined stream into the alkylation unit.

32. The method of claim 1, further comprising:
adjusting a ratio of olefins to paraffins introduced into the alkylation unit by combining at least a portion of a third hydrocarbon stream with at least a portion of the second hydrocarbon stream upstream of the alkylation unit to form a combined stream, wherein the third hydrocarbon stream comprises greater than about 80 percent paraffins by weight; and
introducing the combined stream into the alkylation unit.

33. The method of claim 1, further comprising:
adjusting a ratio of olefins to paraffins introduced into the alkylation unit by combining at least a portion of a third hydrocarbon stream with at least a portion of the second hydrocarbon stream upstream of the alkylation unit to form a combined stream, wherein the third hydrocarbon stream comprises between about 80 percent to about 95 percent paraffins by weight; and
introducing the combined stream into the alkylation unit.

34. The method of claim 1, further comprising:
adjusting a ratio of olefins to paraffins introduced into the alkylation unit by combining at least a portion of a third hydrocarbon stream with at least a portion of the second hydrocarbon stream upstream of the alkylation unit to form a combined stream, wherein the third hydrocarbon stream comprises linear olefins; and
introducing the combined stream into the alkylation unit.

35. The method of claim 1, further comprising:
adjusting a ratio of olefins to paraffins introduced into the alkylation unit by combining at least a portion of a third hydrocarbon stream with at least a portion of the second hydrocarbon stream upstream of the alkylation unit to form a combined stream, wherein at least a portion of the second hydrocarbon stream comprises branched olefins; and
introducing the combined stream into the alkylation unit.

36. The method of claim 1, further comprising:
adjusting a ratio of olefins to paraffins introduced into the alkylation unit by combining at least a portion of a third hydrocarbon stream with at least a portion of the second hydrocarbon stream upstream of the alkylation unit to form a combined stream, wherein at least a portion of the third hydrocarbon stream comprises linear olefins and at least a portion of the second hydrocarbon stream comprises branched olefins; and
introducing the combined stream into the alkylation unit.

37. The method of claim 1, further comprising:
adjusting a ratio of olefins to paraffins introduced into the isomerization unit by combining at least a portion of a paraffinic hydrocarbon stream with at least a portion of the first hydrocarbon stream upstream of the isomerization unit to form a combined stream;
introducing the combined stream into the isomerization unit;
adjusting a ratio of olefins to paraffins introduced into the alkylation unit by combining at least a portion of a third hydrocarbon stream with at least a portion of the second hydrocarbon stream upstream of the alkylation unit to form a combined stream; and
introducing the combined stream into the alkylation unit.

38. The method of claim 1, wherein the dehydrogenation unit is operated at a temperature between about 300° C. and about 700° C.

39. The method of claim 1, wherein the dehydrogenation unit is operated at a pressure between about 1.0 atmosphere and about 15 atmospheres.

40. The method of claim 1, wherein a residence time of at least a portion of the unreacted hydrocarbons stream in the dehydrogenation unit is such that the conversion level of the paraffins in the unreacted hydrocarbons stream composition to olefins is less than about 50 mole percent.

41. The method of claim 1, wherein introducing the olefinic hydrocarbon stream into the isomerization unit comprises combining at least a portion of the olefinic hydrocarbon stream with at least a portion of the first hydrocarbon stream to produce a combined stream upstream from the isomerization unit, and introducing at least a portion of the combined stream into the isomerization unit.

42. The method of claim 1, further comprising separating non-converted paraffins from the olefinic stream and introducing at least a portion of the non-converted paraffins separated from the olefinic stream into the dehydrogenation unit.

43. The method of claim 1, further comprising introducing at least a portion of the alkyl aromatic hydrocarbon stream into a sulfonation unit, wherein the sulfonation unit is configured to sulfonate at least a portion of the alkyl aromatic hydrocarbons in the alkyl aromatic hydrocarbon stream to produce alkyl aromatic sulfonates, wherein at least a portion of the alkyl aromatic sulfonates produced comprise branched alkyl aromatic sulfonates.

44. A method for the production of alkyl aromatic hydrocarbons, comprising:
introducing a first hydrocarbon stream comprising olefins and paraffins into a dehydrogenation-isomerization unit, wherein the dehydrogenation-isomerization unit is configured to dehydrogenate at least a portion of the paraffins in the first hydrocarbon stream to olefins, and wherein the dehydrogenation-isomerization unit is further configured to isomerize at least a portion of linear olefins to branched olefins, and wherein at least a portion of the unreacted components of the first hydrocarbon stream and at least a portion of the products of the dehydrogenation and isomerization reactions form a second hydrocarbon stream, the second hydrocarbon stream comprising olefins and paraffins, and wherein at least a portion of the olefins in the second hydrocarbon stream are branched olefins; and
introducing at least a portion of the second hydrocarbon stream and aromatic hydrocarbons into an alkylation unit, wherein the alkylation unit is configured to alkylate at least a portion of the aromatic hydrocarbons with at least a portion of the olefins in the second hydrocarbon stream to produce alkyl aromatic hydrocarbons, wherein at least a portion of the produced alkyl aromatic hydrocarbons comprise a branched alkyl group.

45. The method of claim 44, wherein the first hydrocarbon stream is produced from an olefin oligomerization process.

46. The method of claim 44, wherein the first hydrocarbon stream is produced from a Fischer-Tropsch process.

47. The method of claim 44, wherein the first hydrocarbon stream comprises olefins and paraffins having carbon numbers from 10 to 16.

48. The method of claim 44, wherein the first hydrocarbon stream comprises olefins and paraffins having carbon numbers from 10 to 13.

49. The method of claim 44, wherein the first hydrocarbon stream comprises an olefin content between about 1 percent and about 50 percent of the total amount of hydrocarbons in the first hydrocarbon stream.

50. The method of claim 44, wherein the first hydrocarbon stream comprises about 80 percent paraffins.

51. The method of claim 44, wherein the dehydrogenation-isomerization unit is operated at a temperature of between about 300° C. and about 500° C.

52. The method of claim 44, wherein the dehydrogenation-isomerization unit is configured to operate at a pressure of between about 0.10 atmosphere and about 15 atmospheres.

53. The method of claim 44, wherein a residence time at least a portion of the unreacted hydrocarbons stream in the dehydrogenation-isomerization unit is such that the conversion level of the paraffins in the unreacted hydrocarbons stream composition to olefins is less than about 50 mole percent.

54. The method of claim 44, wherein a portion of the branched olefins comprise an average number of branches per total olefin molecules of at least 0.7.

55. The method of claim 44, wherein a portion of the branched olefins comprise methyl and ethyl branches.

56. The method of claim 44, wherein a portion of the branched olefins comprise an average number of branches per total olefin molecules of less than about 2.5.

57. The method of claim 44, wherein a portion of the branched olefins comprise an average number of branches per total olefin molecules of between about 0.7 and about 2.0.

58. The method of claim 44, wherein a portion of the branched olefins comprise an average number of branches per total olefin molecules of between about 0.7 and about 1.5.

59. The method of claim 44, wherein a portion of the branched olefins comprise an average number of branches per total olefin molecules of between about 1.0 and about 1.5.

60. The method of claim 44, wherein greater than about 50 percent of the branched groups on the branched olefins are methyl groups.

61. The method of claim 44, wherein less than about 10 percent of the branched groups on the branched olefins are ethyl groups.

62. The method of claim 44, wherein less than about 5 percent of the branched groups on the branched olefins are neither methyl or ethyl groups.

63. The method of claim 44, wherein the branched olefins have less than about 0.5 percent aliphatic quaternary carbon atoms.

64. The method of claim 44, wherein the branched olefins have less than about 0.3 percent aliphatic quaternary carbon atoms.

65. The method of claim 44, wherein the alkylation unit is configured to produce greater than about 50 percent of monoalkylated aromatic hydrocarbons.

66. The method of claim 44, wherein the alkylation unit is configured to produce greater than about 85 percent of monoalkylated aromatic hydrocarbons.

67. The method of claim 44, wherein a molar ratio of the aromatic hydrocarbons to the branched olefins is between about 0.1 to about 2.0 in the alkylation unit.

68. The method of claim 44, wherein the alkylation unit is operated at a reaction temperature between about 30° C. and about 300° C.

69. The method of claim 44, further comprising adjusting a ratio of olefins to paraffins introduced into the alkylation unit by adding at least a portion of a third hydrocarbon stream into the alkylation unit.

70. The method of claim 44, further comprising adjusting a ratio of olefins to paraffins introduced into the alkylation unit by adding at least a portion of a third hydrocarbon stream into the alkylation unit, wherein the third hydrocarbon stream comprises greater than about 90 percent paraffins by weight.

71. The method of claim 44, further comprising adjusting a ratio of olefins to paraffins introduced into the alkylation unit by combining at least a portion of a third hydrocarbon stream with at least a portion of the second hydrocarbon stream upstream of the alkylation unit to form a combined stream and introducing the combined stream into the alkylation unit.

72. The method of claim 44, further comprising adjusting a ratio of olefins to paraffins introduced into the alkylation unit by combining at least a portion of a third hydrocarbon stream with at least a portion of the second hydrocarbon stream upstream of the alkylation unit to form a combined stream, wherein the third hydrocarbon stream comprises greater than about 90 percent paraffins by weight; and introducing the mixed stream into the alkylation unit.

73. The method of claim 44, wherein the branched alkyl groups of the alkyl aromatic hydrocarbons comprise 0.5 percent or less aliphatic quaternary carbon atoms, and an average number of branches per alkyl group of at least 0.7, the branches comprising methyl and ethyl branches.

74. The method of claim 44, further comprising:
forming an alkylation reaction stream wherein the alkylation reaction stream comprises at least a portion of the unreacted components of the second hydrocarbon stream, at least a portion of the aromatic hydrocarbons and at least a portion of the produced alkyl aromatic hydrocarbons;
separating alkyl aromatic hydrocarbons from the alkylation reaction stream to produce an unreacted hydrocarbons stream and an alkyl aromatic hydrocarbon stream; the unreacted hydrocarbons stream comprising at least a portion of the unreacted components of the second hydrocarbon stream and aromatic hydrocarbons;
separating at least a portion of the paraffins and at least a portion of the olefins from the unreacted hydrocarbons stream to produce an aromatic hydrocarbons stream and a paraffins and unreacted olefins stream; and
introducing at least a portion of the paraffins and unreacted olefins stream into the dehydrogenation-isomerization unit.

75. The method of claim 74, wherein introducing at least a portion of the paraffins and unreacted olefins stream into the dehydrogenation-isomerization unit comprises combining at least a portion of the paraffins and unreacted olefins stream with at least a portion of the first hydrocarbon stream to produce a combined stream upstream of the dehydrogenation-isomerization unit and introducing at least a portion of the combined stream into the dehydrogenation-isomerization unit.

76. The method of claim 74, further comprising introducing at least a portion of the alkyl aromatic hydrocarbons stream into a sulfonation unit, wherein the sulfonation unit is configured to sulfonate at least a portion of the alkyl aromatic hydrocarbons in the alkyl aromatic hydrocarbons stream to produce alkyl aromatic sulfonates, wherein at least a portion of the alkyl aromatic sulfonates produced comprise branched alkyl aromatic sulfonates.

77. The method of claim 44, wherein the dehydrogenation-isomerization unit comprises a dehydrogenation-isomerization catalyst configured to catalyze both dehydrogenation reactions and isomerization reactions in the dehydrogenation-isomerization unit, the catalyst comprising a hydrogen form of a zeolite have a ferrierite isotypic framework structure, a binder, a coke-oxidizing compound, and a paraffin dehydrogenation promoting compound.

78. The method of claim 77, wherein the coke-oxidizing compound comprises chrome oxide, iron oxide or noble metals or mixtures thereof.

79. The method of claim 77, wherein the coke-oxidizing compound comprises a platinum, palladium, iridium, ruthenium, osmium or rhodium or mixtures thereof.

80. The method of claim 77, wherein the coke-oxidizing compound is a noble metal.

81. The method of claim 77, wherein the paraffin dehydrogenation promoting compound is a noble metal.

82. The method of claim 77, wherein the paraffin dehydrogenation promoting compound is platinum.

83. The method of claim 77, wherein the binder is selected from natural clays, alumina and silica-alumina.

84. The method of claim 77, wherein the binder is alumina.

85. The method of claim 44, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, and wherein the second reaction zone is configured to isomerize at least a portion of linear olefins to branched olefins, and wherein at least a portion of the unreacted components of the first hydrocarbon stream and at least a portion of the products of the dehydrogenation and isomerization reactions form a second hydrocarbon stream.

86. The method of claim 44, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, wherein the second reaction zone is configured to isomerize at least a portion of linear olefins to branched olefins; and wherein the first reaction zone is operated at a temperature of between about 300° C. and about 600° C.

87. The method of claim 44, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, wherein the second reaction zone is configured to isomerize at least a portion of linear olefins to branched olefins; and wherein the first reaction zone is operated at a temperature of between about 350° C. and about 550° C.

88. The method of claim 44, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, wherein the second reaction zone is configured to isomerize at least a portion of linear olefins to branched olefins; and wherein the first reaction zone is operated at a total reaction pressure between about 1.0 atmospheres and about 5.0 atmospheres.

89. The method of claim 44, further comprising introducing hydrogen into the first hydrocarbon stream.

90. The method of claim 44, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, wherein the second reaction zone is configured to isomerize at least a portion of linear olefins to branched olefins; and wherein a residence time of at least a portion of the first hydrocarbon stream in the first reaction zone is such that the conversion level of the paraffins to olefins is less than about 50 mole percent.

91. The method of claim 85, further comprising introducing at least a portion of the first hydrocarbon stream exiting the first reaction zone into a heat exchanger, wherein the heat exchanger is configured to remove heat from a portion of the first hydrocarbon stream before it enters the second reaction zone.

92. The method of claim 44, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, wherein the second reaction zone is configured to isomerize at least a portion of linear olefins to branched olefins; and wherein the second reaction zone is operated at a temperature range of about 200° C. to about 500° C.

93. The method of claim 44, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, wherein the second reaction zone is configured to isomerize at least a portion of linear olefins to branched olefins; and wherein the second reaction zone is operated at a hydrocarbon partial pressure between about 0.1 atmosphere and about 10 atmospheres.

94. The method of claim 44, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, wherein the second reaction zone is configured to isomerize at least a portion of linear olefins to branched olefins; and wherein the second reaction zone is operated at a hydrocarbon partial pressure between about 0.5 atmosphere and about 2 atmospheres.

95. The method of claim 44, wherein the dehydrogenation-isomerization unit comprises a stacked bed catalyst configuration, wherein the stacked bed catalyst comprises a dehydrogenation catalyst and an isomerization catalyst.

96. A method for the production of alkyl aromatic hydrocarbons, comprising:
introducing a first hydrocarbon stream comprising olefins and paraffins into a dimerization unit, wherein the dimerization unit is configured to dimerize at least a portion of the olefins in the first hydrocarbon stream to produce dimerized olefins, and wherein at least a portion of the unreacted components of the first hydrocarbon stream and at least a portion of the produced dimerized olefins form a second hydrocarbon stream, and wherein at least a portion of the dimerized olefins are branched olefins; and wherein the dimerization unit comprises a dimerization catalyst configured to dimerize at least a portion of the olefins, the catalyst comprising nickel oxide; and
introducing at least a portion of the second hydrocarbon stream and aromatic hydrocarbons into an alkylation unit, wherein the alkylation unit is configured to alkylate at least a portion of the aromatic hydrocarbons with at least a portion of the olefins from the second hydrocarbon stream to produce alkyl aromatic hydrocarbons, wherein a least a portion of the produced alkyl aromatic hydrocarbons comprise a branched alkyl group.

97. The method of claim 96, wherein the first hydrocarbon stream is produced from a Fischer-Tropsch process.

98. The method of claim 96, wherein the first hydrocarbon stream comprises olefins having carbon numbers from 4 to 8.

99. The method of claim 96, wherein the first hydrocarbon stream comprises olefins having carbon numbers from 4 to 6.

100. The method of claim 96, wherein the first hydrocarbon stream comprises between about 50 percent and about 99 percent olefins.

101. The method of claim 96, wherein the first hydrocarbon stream comprises olefins, wherein the olefin composition comprises linear and branched olefins.

102. The method of claim 96, wherein the dimerization unit is operated at temperature between about 120° C. and about 200° C.

103. The method of claim 96, wherein the dimerization unit is operated at temperature between about 150° C. and about 165° C.

104. The method of claim 96, wherein the dimerization unit is configured to produce greater than 20 percent of a branched dimer compound.

105. The method of claim 96, wherein the second hydrocarbon stream comprises olefins, wherein the olefin composition comprises linear and branched olefins having carbon numbers from 10 to 16.

106. The method of claim 96, wherein the second hydrocarbon stream-comprises olefins, wherein the olefin composition comprises linear and branched olefins having carbon numbers from 8 to 12.

107. The method of claim 96, wherein the second hydrocarbon stream comprises olefins, —wherein the olefin composition comprises linear and branched olefins having carbon numbers from 10 to 16 and wherein at least a portion of the branched olefins comprises methyl and ethyl branches.

108. The method of claim 96, wherein a portion of the branched olefins comprise an average number of branches per total olefin molecules of at least 0.7.

109. The method of claim 96, wherein a portion of the branched olefins comprise methyl and ethyl branches.

110. The method of claim 96, wherein a portion of the branched olefins comprise an average number of branches per total olefin molecules of less than about 2.5.

111. The method of claim 96, wherein a portion of the branched olefins comprise an average number of branches per total olefin molecules of between about 0.7 and about 2.0.

112. The method of claim 96, wherein a portion of the branched olefins comprise an average number of branches per total olefin molecules of between about 0.7 and about 1.5.

113. The method of claim 96, wherein a portion of the branched olefins comprise an average number of branches per total olefin molecules of between about 1.0 and about 1.5.

114. The method of claim 96, wherein greater than about 50 percent of the branched groups on the branched olefins are methyl groups.

115. The method of claim 96, wherein less than about 10 percent of the branched groups on the branched olefins are ethyl groups.

116. The method of claim 96, wherein less than about 5 percent of the branched groups on the branched olefins are neither methyl or ethyl groups.

117. The method of claim 96, wherein the branched olefins have less than about 0.5 percent aliphatic quaternary carbon atoms.

118. The method of claim 96, wherein the branched olefins have less than about 0.3 percent aliphatic quaternary carbon atoms.

119. The method of claim 96, further comprising:
separating the produced dimerized olefins from the second hydrocarbon stream to form a produced dimerized olefins stream and a paraffins and unreacted olefins stream, wherein the paraffins and unreacted olefins stream comprises hydrocarbons of a carbon number less than 7; and
introducing at least a portion of the paraffins and unreacted olefins stream into the dimerization unit.

120. The method of claim 96, further comprising adjusting a ratio of olefins to paraffins introduced into the alkylation unit by adding at least a portion of a third hydrocarbon stream into the alkylation unit.

121. The method of claim 96, further comprising adjusting a ratio of olefins to paraffins introduced into the alkylation unit by adding at least a portion of a third hydrocarbon stream into the alkylation unit wherein the third hydrocarbon stream comprises greater than about 90 percent paraffins by weight.

122. The method of claim 96, further comprising adjusting a ratio of olefins to paraffins introduced into the alkylation unit by combining at least a portion of a third hydrocarbon stream with at least a portion of the second hydrocarbon stream upstream of the alkylation unit to form a combined stream and introducing the combined stream into the alkylation unit.

123. The method of claim 96, further comprising adjusting a ratio of olefins to paraffins introduced into the alkylation unit by combining at least a portion of a third hydrocarbon stream with at least a portion of the second hydrocarbon stream upstream of the alkylation unit to form a combined stream and introducing the combined stream into the alkylation unit, and wherein the third hydrocarbon stream comprises greater than about 90 percent paraffins by weight.

124. The method of claim 96, further comprising adjusting a ratio of olefins to paraffins introduced into the alkylation unit by combining at least a portion of a third hydrocarbon stream with at least a portion of the second hydrocarbon stream upstream of the alkylation unit to form a combined stream and introducing the mixed stream into the alkylation unit, and wherein the third hydrocarbon stream comprises paraffins and olefins having a carbon number from 10 to 16.

125. The method of claim 96, wherein the alkylation unit is configured to produce greater than about 50 percent of monoalkylated aromatic hydrocarbons.

126. The method of claim 96, wherein the alkylation unit is configured to produce greater than about 85 percent of monoalkylated aromatic hydrocarbons.

127. The method of claim 96, wherein a molar ratio of the aromatic hydrocarbons to the branched olefins is between about 0.1 to about 2.0 in the alkylation unit.

128. The method of claim 96, wherein the alkylation unit is operated at a reaction temperature between about 30° C. and about 300° C.

129. The method of claim 96, wherein the branched alkyl groups of the alkyl aromatic hydrocarbons comprise 0.5 percent or less aliphatic quaternary carbon atoms, and an average number of branches per alkyl group of at least 0.7, the branches comprising methyl and ethyl branches.

130. The method of claim 96, further comprising:
forming an alkylation reaction stream wherein the alkylation reaction stream comprises at least a portion of the aromatic hydrocarbons, at least a portion of the unreacted components of the second hydrocarbon stream and at least a portion of the produced alkyl aromatic hydrocarbons;
separating alkyl aromatic hydrocarbons from the alkylation reaction stream to produce an unreacted hydrocarbons stream and an alkyl aromatic hydrocarbon stream; the unreacted hydrocarbons stream comprising at least a portion of the unreacted components of the second hydrocarbon stream and at least a portion of the aromatic hydrocarbons;
separating at least a portion of the paraffins and at least a portion of the olefins from the unreacted hydrocarbons stream to produce an aromatic hydrocarbons stream and a paraffins and unreacted olefins stream;
introducing at least a portion of the aromatic hydrocarbons stream into the alkylation unit.

131. The method of claim 130, further comprising separating olefins from the paraffins and unreacted olefins stream to produce an olefinic stream, wherein the olefins in the olefinic stream have a carbon number from 4 to 8 and introducing at least a portion of the olefinic stream into the dimerization unit.

132. The method of claim 96, further comprising introducing at least a portion of the alkyl aromatic hydrocarbons stream into a sulfonation unit, wherein the sulfonation unit is configured to sulfonate at least a portion of the alkyl aromatic hydrocarbons in the alkyl aromatic hydrocarbons stream to produce alkyl aromatic sulfonates and wherein at least a portion of the alkyl aromatic sulfonates produced comprise branched alkyl aromatic sulfonates.

133. A method for the production of alkyl aromatic hydrocarbons, comprising:
introducing a first hydrocarbon stream comprising olefins and paraffins into a dimerization unit, wherein the dimerization unit is configured to dimerize at least a portion of the olefins in the first hydrocarbon stream to produce dimerized olefins, and wherein at least a portion of the unreacted components of the first hydrocarbon stream and the produced dimerized olefins form a second hydrocarbon stream, and wherein at least a portion of the dimerized olefins are branched olefins;
introducing at least a portion of the second hydrocarbon stream and aromatic hydrocarbons into an alkylation unit;
introducing a third hydrocarbon stream into an isomerization unit, wherein the isomerization unit is configured to isomerize at least a portion of linear olefins in the third hydrocarbon stream to branched olefins, and wherein at least a portion of the unreacted components of the third hydrocarbon stream and the produced branched olefins form a fourth hydrocarbon stream,
introducing at least a portion of the fourth hydrocarbon stream into the alkylation unit, wherein the alkylation unit is configured to alkylate at least a portion of the aromatic hydrocarbons with at least a portion of the olefins from the second hydrocarbon stream and the fourth hydrocarbon stream to produce alkyl aromatic hydrocarbons, wherein a least a portion of the produced alkyl aromatic hydrocarbons comprise a branched alkyl group.

134. The method of claim 133, wherein the first hydrocarbon stream is produced from a Fischer-Tropsch process.

135. The method of claim 133, wherein the first hydrocarbon stream comprises olefins.

136. The method of claim 133, wherein the second hydrocarbon stream comprises olefins having a carbon number from 10 to 16.

137. The method of claim 133, wherein a portion of the branched olefins comprise an average number of branches per total olefin molecules of at least 0.7.

138. The method of claim 133, wherein a portion of the branched olefins comprise methyl and ethyl branches.

139. The method of claim 133, wherein a portion of the branched olefins comprise an average number of branches per total olefin molecules of less than about 2.5.

140. The method of claim 133, wherein a portion of the branched olefins comprise an average number of branches per total olefin molecules of between about 0.7 and about 2.0.

141. The method of claim 133, wherein a portion of the branched olefins comprise an average number of branches per total olefin molecules of between about 0.7 and about 1.5.

142. The method of claim 133, wherein a portion of the branched olefins comprise an average number of branches per total olefin molecules of between about 1.0 and about 1.5.

143. The method of claim 133, wherein greater than about 50 percent of the branched groups on the branched olefins are methyl groups.

144. The method of claim 133, wherein less than about 10 percent of the branched groups on the branched olefins are ethyl groups.

145. The method of claim 133, wherein less than about 5 percent of the branched groups on the branched olefins are neither methyl or ethyl groups.

146. The method of claim 133, wherein the branched olefins have less than about 0.5 percent aliphatic quaternary carbon atoms.

147. The method of claim 133, wherein the branched olefins have less than about 0.3 percent aliphatic quaternary carbon atoms.

148. The method of claim 133, further comprising:
separating the produced dimerized olefins from the second hydrocarbon stream to form a produced dimerized olefins stream and a paraffins and unreacted olefins stream, wherein the paraffins and unreacted olefins stream comprises hydrocarbons of a carbon number less than 7; and
introducing at least a portion of the paraffins and unreacted olefins stream into the dimerization unit.

149. The method of claim 133, wherein the alkylation unit is configured to produce greater than about 50 percent of monoalkylated aromatic hydrocarbons.

150. The method of claim 133, wherein the alkylation unit is configured to produce greater than about 85 percent of monoalkylated aromatic hydrocarbons.

151. The method of claim 133, wherein a molar ratio of the aromatic hydrocarbons to the branched olefins is between about 0.1 to about 2.0 in the alkylation unit.

152. The method of claim 133, wherein the alkylation unit is operated at a reaction temperature between about 30° C. and about 300° C.

153. The method of claim 133, wherein the branched alkyl groups of the alkyl aromatic hydrocarbons comprise 0.5 percent or less aliphatic quaternary carbon atoms, and an average number of branches per alkyl group of at least 0.7, the branches comprising methyl and ethyl branches.

154. The method of claim 133, wherein introducing at least a portion of the fourth hydrocarbon stream into the alkylation unit comprises combining at least a portion of the fourth hydrocarbon stream with at least a portion of the second hydrocarbon stream to produce a combined stream upstream of the alkylation unit.

155. The method of claim 133, further comprising adjusting a ratio of olefins to paraffins introduced into the alkylation unit by adding at least a portion of a fifth hydrocarbon stream into the alkylation unit.

156. The method of claim 133, further comprising adjusting a ratio of olefins to paraffins introduced into the alkylation unit by adding at least a portion of a fifth hydrocarbon stream into the alkylation unit and wherein the fifth hydrocarbon stream comprises greater than about 90 percent paraffins by weight.

157. The method of claim 133, further comprising adjusting a ratio of olefins to paraffins introduced into the alkylation unit by adding at least a portion of a fifth hydrocarbon stream to at least a portion of the second hydrocarbon stream upstream of the alkylation unit.

158. The method of claim 133, further comprising adjusting a ratio of olefins to paraffins introduced into the alkylation unit by adding at least a portion of a fifth hydrocarbon stream to at least a portion of the second hydrocarbon stream upstream of the alkylation unit to form a combined stream and wherein the fifth hydrocarbon stream comprises greater than about 90 percent paraffins by weight.

159. The method of claim 133, further comprising:
forming an alkylation reaction stream wherein the alkylation reaction stream comprises at least a portion of the aromatic hydrocarbons, at least a portion of the unreacted components of the second hydrocarbon stream and at least a portion of the produced alkyl aromatic hydrocarbons;
separating alkyl aromatic hydrocarbons from the alkylation reaction stream to produce an unreacted hydrocarbons stream and an alkyl aromatic hydrocarbon stream; the unreacted hydrocarbons stream comprising at least a portion of the unreacted components of the second hydrocarbon stream and at least a portion of the aromatic hydrocarbons;
separating at least a portion of the paraffins and at least a portion of the olefins from the unreacted hydrocarbons stream to produce an aromatic hydrocarbons stream and a paraffins and unreacted olefins stream; and
introducing at least a portion of the paraffins and unreacted olefins stream into the isomerization unit.

160. The method of claim 159, further comprising introducing at least a portion of the alkyl aromatic hydrocarbons stream into a sulfonation unit, wherein the sulfonation unit is configured to sulfonate at least a portion of the alkyl aromatic hydrocarbons in the alkyl aromatic hydrocarbons stream to produce alkyl aromatic sulfonates and wherein at least a portion of the alkyl aromatic sulfonates produced comprise branched alkyl aromatic sulfonates.

161. The method of claim 159, further comprising separating the olefins from the paraffins and unreacted olefins stream to produce an olefinic stream, wherein the average carbon number of the olefins in the olefinic stream is from 4 to 8 and introducing at least a portion of the olefinic stream into the dimerization unit.

162. A method for the production of alkyl aromatic hydrocarbons, comprising:
introducing a first hydrocarbon stream comprising olefins and paraffins into a hydrogenation unit, wherein the hydrogenation unit is configured to hydrogenate at least a portion of olefins in the first hydrocarbon stream to paraffins, and wherein at least a portion of the unreacted components of the first hydrocarbon stream and at least a portion of the hydrogenated olefins form a second hydrocarbon stream,
introducing the second hydrocarbon stream into a dehydrogenation unit, wherein the dehydrogenation unit is configured to dehydrogenate at least a portion of paraffins in the second hydrocarbon stream to olefins, and wherein at least a portion of the unreacted components of the second hydrocarbon stream and at least a portion of the olefins from the dehydrogenation process form a third hydrocarbon stream,
introducing the third hydrocarbon stream into an isomerization unit, wherein the isomerization unit is configured to isomerize at least a portion of olefins in the third hydrocarbon stream to branched olefins, and wherein at least a portion of the unreacted components of the third hydrocarbon stream and at least a portion of the produced branched olefins form a fourth hydrocarbon stream;
introducing at least a portion of the fourth hydrocarbon stream and aromatic hydrocarbons into an alkylation unit, wherein the alkylation unit is configured to alkylate at least a portion of the aromatic hydrocarbons with at least a portion of the olefins in the fourth hydrocarbon stream to produce alkyl aromatic hydrocarbons, wherein at least a portion of the produced alkyl aromatic hydrocarbons comprise a branched alkyl group.

163. The method of claim 162, wherein the first hydrocarbon stream is produced from a Fischer-Tropsch process.

164. The method of claim 162, wherein the first hydrocarbon stream comprises olefins and paraffins having carbon numbers from 10 to 13.

165. The method of claim 162, wherein the first hydrocarbon stream comprises olefins and paraffins having carbon numbers from 10 to 16.

166. The method of claim 162, wherein the hydrogenation unit is operated at a temperature between about 175° C. and about 250° C.

167. The method of claim 162, wherein the hydrogenation unit is operated at a hydrogen flow rate between about 250 NL/L/hr and about 5000 NL/L/hr.

168. The method of claim 162, wherein the hydrogenation unit is operated at a pressure between about 10 atmospheres and about 50 atmospheres.

169. The method of claim 162, wherein the dehydrogenation unit is operated at a temperature between about 300° C. and about 700° C.

170. The method of claim 162, wherein the dehydrogenation unit is operated at a pressure between about 1.0 atmosphere and about 15 atmospheres.

171. The method of claim 162, wherein a residence time of at least a portion of the unreacted hydrocarbons stream in the dehydrogenation unit is such that the conversion level of the paraffins in the unreacted hydrocarbons stream composition to olefins is less than about 50 mole percent.

172. The method of claim 162, wherein the isomerization unit is operated at a reaction temperature between about 200° C. and about 500° C.

173. The method of claim 162, wherein the isomerization unit is operated at a reaction pressure between about 0.1 atmosphere and about 10 atmospheres.

174. The method of claim 162, wherein at least a portion of the branched olefins comprise an average number of branches per total olefin molecules of at least about 0.7.

175. The method of claim 162, wherein at least a portion of the branched olefins comprise methyl and ethyl branches.

176. The method of claim 162, wherein at least a portion of the branched olefins comprise an average number of branches per total olefin molecules of less than about 2.5.

177. The method of claim 162, wherein at least a portion of the branched olefins comprise an average number of branches per total olefin molecules of between about 0.7 and about 2.0.

178. The method of claim 162, wherein at least a portion of the branched olefins comprise an average number of branches per total olefin molecules of between about 0.7 and about 1.5.

179. The method of claim 162, wherein at least a portion of the branched olefins comprise an average number of branches per total olefin molecules of between about 1.0 and about 1.5.

180. The method of claim 162, wherein greater than about 50 percent of the branched groups on the branched olefins are methyl groups.

181. The method of claim 162, wherein less than about 10 percent of the branched groups on the branched olefins are ethyl groups.

182. The method of claim 162, wherein less than about 5 percent of the branched groups on the branched olefins are neither methyl or ethyl groups.

183. The method of claim 162, wherein the branched olefins have less than about 0.5 percent aliphatic quaternary carbon atoms.

184. The method of claim 162, wherein the branched olefins have less than about 0.3 percent aliphatic quaternary carbon atoms.

185. The method of claim 162, wherein the alkylation unit is configured to produce greater than about 50 percent of monoalkylated aromatic hydrocarbons.

186. The method of claim 162, wherein the alkylation unit is configured to produce greater than about 85 percent of monoalkylated aromatic hydrocarbons.

187. The method of claim 162, wherein a molar ratio of the aromatic hydrocarbons to the branched olefins is between about 0.1 and about 2.0 in the alkylation unit.

188. The method of claim 162, wherein the alkylation unit is operated at a reaction temperature between about 30° C. and about 300° C.

189. The method of claim 162, wherein the aromatic hydrocarbons comprise benzene.

190. The method of claim 162, wherein the alkyl aromatic hydrocarbons comprise alkylbenzenes.

191. The method of claim 162, wherein the branched alkyl groups of the alkyl aromatic hydrocarbons comprise 0.5 percent or less aliphatic quaternary carbon atoms, and an average number of branches per alkyl group of at least 0.7, the branches comprising methyl and ethyl branches.

192. The method of claim 162, further comprising:
forming an alkylation reaction stream wherein the alkylation reaction stream comprises at least a portion of the aromatic hydrocarbons, at least a portion of the unreacted components of the fourth hydrocarbon stream and at least a portion of the produced alkyl aromatic hydrocarbons;
separating alkyl aromatic hydrocarbons from the alkylation reaction stream to produce an unreacted hydrocarbons stream and an alkyl aromatic hydrocarbon stream; the unreacted hydrocarbons stream comprising at least a portion of the unreacted components of the fourth hydrocarbon stream and at least a portion of the aromatic hydrocarbons;
separating at least a portion of the paraffins and at least a portion of the olefins from the unreacted hydrocarbons stream to produce an aromatic hydrocarbons stream and a paraffins and unreacted olefins stream; and
introducing at least a portion of the paraffins and unreacted olefins stream into the dehydrogenation unit.

193. The method of claim 192, wherein introducing at least a portion of the paraffins and unreacted olefins stream into the dehydrogenation unit comprises combining at least a portion of the paraffins and unreacted olefins stream with at least a portion of the second hydrocarbon stream to produce a combined stream upstream of the dehydrogenation unit and introducing at least a portion of the combined stream into the dehydrogenation unit.

194. The method of claim 192, further comprising: introducing at least a portion of the alkyl aromatic hydrocarbon stream into a sulfonation unit, wherein the sulfonation unit is configured to sulfonate at least a portion of the alkyl aromatic hydrocarbons in the alkyl aromatic hydrocarbon stream to produce alkyl aromatic sulfonates, wherein at least a portion of the alkyl aromatic sulfonates produced comprise branched alkyl aromatic sulfonates.

195. A method for the production of alkyl aromatic hydrocarbons, comprising:
introducing a first hydrocarbon stream comprising olefins and paraffins into a hydrogenation unit, wherein the hydrogenation unit is configured to hydrogenate at least a portion of olefins in the first hydrocarbon stream to paraffins, and wherein at least a portion of the unreacted components of the first hydrocarbon stream and at least a portion of the hydrogenated olefins form a second hydrocarbon stream;
introducing the second hydrocarbon stream into a dehydrogenation unit, wherein the dehydrogenation unit is configured to dehydrogenate at least a portion of paraffins in the second hydrocarbon stream to olefins, and wherein at least a portion of the unreacted components of the second hydrocarbon stream and at least a portion of the olefins from the dehydrogenation process form a third hydrocarbon stream,
introducing the third hydrocarbon stream into a dimerization unit, wherein the dimerization unit is configured to dimerize at least a portion of the olefins in the third hydrocarbon stream to produce dimerized olefins, and wherein at least a portion of the unreacted components of the third hydrocarbon stream and the produced dimerized olefins form a fourth hydrocarbon stream, and wherein at least a portion of the dimerized olefins are branched olefins;
introducing at least a portion of the fourth hydrocarbon stream and aromatic hydrocarbons into an alkylation unit; wherein the alkylation unit is configured to alkylate at least a portion of the aromatic hydrocarbons with at least a portion of the olefins in the fourth hydrocarbon stream to produce alkyl aromatic hydrocarbons, wherein at least a portion of the produced alkyl aromatic hydrocarbons comprise a branched alkyl group.

196. The method of claim 195, wherein the first hydrocarbon stream is produced from a Fischer-Tropsch process.

197. The method of claim 195, wherein the first hydrocarbon stream comprises olefins and paraffins having carbon numbers from 4 to 8.

198. The method of claim 195, wherein the first hydrocarbon stream comprises olefins and paraffins having carbon numbers from 4 to 6.

199. The method of claim 195, wherein the hydrogenation unit is operated at a temperature between about 175° C. and about 250° C.

200. The method of claim 195, wherein the hydrogenation unit is operated at a hydrogen flow rate between about 250 NL/L/hr and about 5000 NL/L/hr.

201. The method of claim 195, wherein the hydrogenation unit is operated at a pressure between about 10 atmospheres and about 50 atmospheres.

202. The method of claim 195, wherein the dehydrogenation unit is operated at a temperature between about 300° C. and about 700° C.

203. The method of claim 195, wherein the dehydrogenation unit is operated at a pressure between about 1.0 atmosphere and about 15 atmospheres.

204. The method of claim 195, wherein a residence time at least a portion of the unreacted hydrocarbons stream in the dehydrogenation unit is such that the conversion level of the paraffins in the unreacted hydrocarbons stream composition to olefins is less than about 50 mole percent.

205. The method of claim 195, wherein the dimerization unit is operated at temperature between about 120° C. and about 200° C.

206. The method of claim 195, wherein the dimerization unit is operated at temperature between about 150° C. and about 165° C.

207. The method of claim 195, wherein the dimerization unit is configured to produce greater than 20 percent of a branched dimer.

208. The method of claim 195, wherein at least a portion of the branched olefins comprise an average number of branches per total olefin molecules of at least 0.7.

209. The method of claim 195, wherein at least a portion of the branched olefins comprise methyl and ethyl branches.

210. The method of claim 195, wherein at least a portion of the branched olefins comprise an average number of branches per total olefin molecules of less than about 2.5.

211. The method of claim 195, wherein at least a portion of the branched olefins comprise an average number of branches per total olefin molecules of between about 0.7 and about 2.0.

212. The method of claim 195, wherein at least a portion of the branched olefins comprise an average number of branches per total olefin molecules of between about 0.7 and about 1.5.

213. The method of claim 195, wherein at least a portion of the branched olefins comprise an average number of branches per total olefin molecules of between about 1.0 and about 1.5.

214. The method of claim 195, wherein greater than about 50 percent of the branched groups on the branched olefins are methyl groups.

215. The method of claim 195, wherein less than about 10 percent of the branched groups on the branched olefins are ethyl groups.

216. The method of claim 195, wherein less than about 5 percent of the branched groups on the branched olefins are neither methyl or ethyl groups.

217. The method of claim 195, wherein the branched olefins have less than about 0.5 percent aliphatic quaternary carbon atoms.

218. The method of claim 195, wherein the branched olefins have less than about 0.3 percent aliphatic quaternary carbon atoms.

219. The method of claim 195, wherein the alkylation unit is configured to produce greater than about 50 percent of monoalkylated aromatic hydrocarbons.

220. The method of claim 195, wherein the alkylation unit is configured to produce greater than about 85 percent of monoalkylated aromatic hydrocarbons.

221. The method of claim 195, wherein a molar ratio of the aromatic hydrocarbons to the branched olefins is between about 0.1 and about 2.0 in the alkylation unit.

222. The method of claim 195, wherein the alkylation unit is operated at a reaction temperature between about 30° C. and about 300° C.

223. The method of claim 195, wherein the aromatic hydrocarbons comprise benzene.

224. The method of claim 195, wherein the alkyl aromatic hydrocarbons comprise alkylbenzenes.

225. The method of claim 195, wherein the branched alkyl groups of the alkyl aromatic hydrocarbons comprise 0.5 percent or less aliphatic quaternary carbon atoms, and an average number of branches per alkyl group of at least 0.7, the branches comprising methyl and ethyl branches.

226. The method of claim 195, further comprising:
separating the produced dimerized olefins from the fourth hydrocarbon stream to form a produced dimerized olefins stream and a paraffins and unreacted olefins stream, wherein the paraffins and unreacted olefins stream comprises hydrocarbons of a carbon number less than 7; and
introducing at least a portion of the paraffins and unreacted olefins stream into the dehydrogenation unit.

227. The method of claim 195, further comprising:
forming an alkylation reaction stream wherein the alkylation reaction stream comprises at least a portion of the aromatic hydrocarbons, at least a portion of the unreacted components of the fourth hydrocarbon stream and at least a portion of the produced alkyl aromatic hydrocarbons;
separating alkyl aromatic hydrocarbons from the alkylation reaction stream to produce an unreacted hydrocarbons stream and an alkyl aromatic hydrocarbon stream; the unreacted hydrocarbons stream comprising at least a portion of the unreacted components of the fourth hydrocarbon stream and at least a portion of the aromatic hydrocarbons;
separating at least a portion of the paraffins and at least a portion of the olefins from the unreacted hydrocarbons stream to produce an aromatic hydrocarbons stream and a paraffins and unreacted olefins stream; and
introducing at least a portion of the aromatic hydrocarbons stream into the alkylation unit.

228. The method of claim 227, further comprising, separating olefins from the paraffins and unreacted olefins stream to produce an olefinic stream, wherein the olefins in the olefinic stream have an average carbon number from 4 to 8.

229. The method of claim 227, further comprising, separating olefins from the paraffins and unreacted olefins stream to produce an olefinic stream, wherein the olefins in the olefinic stream have an average carbon number from 4 to 8 and wherein introducing at least a portion of the olefinic stream into the dehydrogenation unit comprises combining at least a portion of the olefinic stream with at least a portion of the second hydrocarbon stream to produce a combined stream upstream of the dehydrogenation unit and introducing at least a portion of the combined stream into the dehydrogenation unit.

230. The method of claim 227, further comprising: introducing at least a portion of the alkyl aromatic hydrocarbon stream into a sulfonation unit, wherein the sulfonation unit is configured to sulfonate at least a portion of the alkyl aromatic hydrocarbons in the alkyl aromatic hydrocarbon stream to produce alkyl aromatic sulfonates, wherein at least a portion of the alkyl aromatic sulfonates produced comprise branched alkyl aromatic sulfonates.

231. The method of claim 195, further comprising:
introducing a fifth hydrocarbon stream into an isomerization unit, wherein the isomerization unit is configured to isomerize at least a portion of linear olefins in the fifth hydrocarbon stream to branched olefins, and wherein at least a portion of the unreacted components of the fifth hydrocarbon stream and the produced branched olefins form a sixth hydrocarbon stream,
introducing at least a portion of the sixth hydrocarbon stream into the alkylation unit, wherein the alkylation unit is configured to alkylate at least a portion of the aromatic hydrocarbons with at least a portion of the olefins from the fourth hydrocarbon stream and the sixth hydrocarbon stream to produce alkyl aromatic hydrocarbons, wherein a least a portion of the produced alkyl aromatic hydrocarbons comprise a branched alkyl group.

232. A method for the production of alkyl aromatic hydrocarbons, comprising:
introducing a first hydrocarbon stream comprising olefins and paraffins into a hydrogenation unit, wherein the hydrogenation unit is configured to hydrogenate at least a portion of olefins in the first hydrocarbon stream to paraffins, and wherein at least a portion of the unreacted components of the first hydrocarbon stream and at least a portion of the hydrogenated olefins form a second hydrocarbon stream,
introducing the second hydrocarbon stream into a dehydrogenation-isomerization unit, wherein the dehydrogenation-isomerization unit is configured to dehydrogenate at least a portion of the paraffins in the first hydrocarbon stream to olefins, and wherein the dehydrogenation-isomerization unit is further configured to isomerize at least a portion of linear olefins to branched olefins, and wherein at least a portion of the unreacted components of the second hydrocarbon stream and at least a portion of the products of the dehydrogenation and isomerization reactions form a third hydrocarbon stream, the third hydrocarbon stream comprising olefins and paraffins, and wherein at least a portion of the olefins in the third hydrocarbon stream are branched olefins;
introducing at least a portion of the third hydrocarbon stream and aromatic hydrocarbons into an alkylation unit, wherein the alkylation unit is configured to alkylate at least a portion of the aromatic hydrocarbons with at least a portion of the olefins in the third hydrocarbon stream to produce alkyl aromatic hydrocarbons, wherein at least a portion of the produced alkyl aromatic hydrocarbons comprise a branched alkyl group.

233. The method of claim 232, wherein the first hydrocarbon stream is produced from a Fischer-Tropsch process.

234. The method of claim 232, wherein the first hydrocarbon stream comprises olefins and paraffins having carbon numbers from 10 to 13.

235. The method of claim 232, wherein the first hydrocarbon stream comprises olefins and paraffins having carbon number from 10 to 16.

236. The method of claim 232, wherein the hydrogenation unit is operated at a temperature between about 175° C. and about 250° C.

237. The method of claim 232, wherein the hydrogenation unit is operated at a hydrogen flow rate between about 250 NL/L/hr and about 5000 NL/L/hr.

238. The method of claim 232, wherein the hydrogenation unit is operated at a pressure between about 10 atmospheres and about 50 atmospheres.

239. The method of claim 232, wherein the dehydrogenation-isomerization unit is operated at a temperature of between about 300° C. and about 500° C.

240. The method of claim 232, wherein the dehydrogenation-isomerization unit is configured to operate at a pressure of between about 0.10 atmosphere and about 15 atmospheres.

241. The method of claim 232, wherein a residence time at least a portion of the unreacted hydrocarbons stream in the dehydrogenation-isomerization unit is such that the conversion level of the paraffins in the unreacted hydrocarbons stream composition to olefins is less than about 50 mole percent.

242. The method of claim 232, wherein a portion of the branched olefins comprise an average number of branches per total olefin molecules of at least 0.7.

243. The method of claim 232, wherein a portion of the branched olefins comprise methyl and ethyl branches.

244. The method of claim 232, wherein a portion of the branched olefins comprise an average number of branches per total olefin molecules of less than about 2.5.

245. The method of claim 232, wherein a portion of the branched olefins comprise an average number of branches per total olefin molecules of between about 0.7 and about 2.0.

246. The method of claim 232, wherein a portion of the branched olefins comprise an average number of branches per total olefin molecules of between about 0.7 and about 1.5.

247. The method of claim 232, wherein a portion of the branched olefins comprise an average number of branches per total olefin molecules of between about 1.0 and about 1.5.

248. The method of claim 232, wherein greater than about 50 percent of the branched groups on the branched olefins are methyl groups.

249. The method of claim 232, wherein less than about 10 percent of the branched groups on the branched olefins are ethyl groups.

250. The method of claim 232, wherein less than about 5 percent of the branched groups on the branched olefins are neither methyl or ethyl groups.

251. The method of claim 232, wherein the branched olefins have less than about 0.5 percent aliphatic quaternary carbon atoms.

252. The method of claim 232, wherein the branched olefins have less than about 0.3 percent aliphatic quaternary carbon atoms.

253. The method of claim 232, wherein the alkylation unit is configured to produce-greater than about 50 percent of monoalkylated aromatic hydrocarbons.

254. The method of claim 232, wherein the alkylation unit is configured to produce greater than about 85 percent of monoalkylated aromatic hydrocarbons.

255. The method of claim 232, wherein a molar ratio of the aromatic hydrocarbons to the branched olefins is between about 0.1 and about 2.0 in the alkylation unit.

256. The method of claim 232, wherein the alkylation unit is operated at a reaction temperature between about 30° C. and about 300° C.

257. The method of claim 232, wherein the aromatic hydrocarbons comprise benzene.

258. The method of claim 232, wherein the alkyl aromatic hydrocarbons comprise alkylbenzenes.

259. The method of claim 232, wherein the branched alkyl groups of the alkyl aromatic hydrocarbons comprise 0.5 percent or less aliphatic quaternary carbon atoms, and an average number of branches per alkyl group of at least 0.7, the branches comprising methyl and ethyl branches.

260. The method of claim 232, further comprising:
forming an alkylation reaction stream wherein the alkylation reaction stream comprises at least a portion of the aromatic hydrocarbons, at least a portion of the unreacted components of the third hydrocarbon stream and at least a portion of the produced alkyl aromatic hydrocarbons;
separating alkyl aromatic hydrocarbons from the alkylation reaction stream to produce an unreacted hydrocarbons stream and an alkyl aromatic hydrocarbon stream; the unreacted hydrocarbons stream comprising at least a portion of the unreacted components of the third hydrocarbon stream and at least a portion of the aromatic hydrocarbons;
separating at least a portion of the paraffins and at least a portion of the olefins from the unreacted hydrocarbons stream to produce an aromatic hydrocarbons stream and a paraffins and unreacted olefins stream; and
introducing at least a portion of the paraffins and unreacted olefins stream into the dehydrogenation-isomerization unit.

261. The method of claim 260, wherein introducing at least a portion of the paraffins and unreacted olefins stream into the dehydrogenation-isomerization unit comprises combining at least a portion of the paraffins and unreacted olefins stream with at least a portion of the second hydrocarbon stream to produce a combined stream upstream of the dehydrogenation-isomerization unit and introducing at least a portion of the combined stream into the dehydrogenation-isomerization unit.

262. The method of claim 232, further comprising: introducing at least a portion of the alkyl aromatic hydrocarbon stream into a sulfonation unit, wherein the sulfonation unit is configured to sulfonate at least a portion of the alkyl aromatic hydrocarbons in the alkyl aromatic hydrocarbon stream to produce alkyl aromatic sulfonates, wherein at least a portion of the alkyl aromatic sulfonates produced comprise branched alkyl aromatic sulfonates.

263. The method of claim 232, wherein the dehydrogenation-isomerization unit comprises a dehydrogenation-isomerization catalyst configured to catalyze both dehydrogenation reactions and isomerization reactions in the dehydrogenation-isomerization unit, the catalyst comprising a hydrogen form of a zeolite have a ferrierite isotypic framework structure, a binder, a coke-oxidizing compound, and a paraffin dehydrogenation promoting compound.

264. The method of claim 263, wherein the coke-oxidizing compound comprises chrome oxide, iron oxide or noble metals or mixtures thereof.

265. The method of claim 263, wherein the coke-oxidizing compound comprises a platinum, palladium, iridium, ruthenium, osmium or rhodium or mixtures thereof.

266. The method of claim 263, wherein the coke-oxidizing compound is a noble metal.

267. The method of claim 263, wherein the paraffin dehydrogenation promoting compound is a noble metal.

268. The method of claim 263, wherein the paraffin dehydrogenation promoting compound is platinum.

269. The method of claim 263, wherein the binder is selected from natural clays, alumina and silica-alumina.

270. The method of claim 263, wherein the binder is alumina.

271. The method of claim 232, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, and wherein the second reaction zone is configured to isomerize at least a portion of linear olefins to branched olefins, and wherein at least a portion of the unreacted components of the first hydrocarbon stream and at least a portion of the products of the dehydrogenation and isomerization reactions form a second hydrocarbon stream.

272. The method of claim 232, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, and wherein the second reaction zone is configured to isomerize at least a portion of linear olefins to branched olefins; and wherein the first reaction zone is operated at a temperature of between about 300° C. and about 600° C.

273. The method of claim 232, wherein the dehydrogenation-isomerization unit comprises t a plurality of zones, wherein the plurality of zones comprises a first reaction zone and a second reaction zone, wherein the first reaction zone is configured to dehydrogenate at least a portion of paraffins to olefins, and wherein the second reaction zone is configured to isomerize at least a portion of linear olefins to branched olefins, and wherein the first reaction zone is operated at a temperature of between about 350° C. and about 550° C.

274. The method of claim 232, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, and wherein the second reaction zone is configured to isomerize at least a portion of linear olefins to branched olefins and wherein the first reaction zone is operated at a total reaction pressure between about 1.0 atmospheres and about 5.0 atmospheres.

275. The method of claim 232, further comprising introducing hydrogen into the first hydrocarbon stream.

276. The method of claim 232, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, wherein the second reaction zone is configured to isomerize at least a portion of linear olefins to branched olefins and wherein a residence time of at least a portion of the first hydrocarbon stream in the first reaction zone is such that the conversion level of the paraffins to olefins is less than about 50 mole percent.

277. The method of claim 271, further comprising introducing at least a portion of the first hydrocarbon stream exiting the first reaction zone into a heat exchanger, wherein the heat exchanger is configured to remove heat from a portion of the first hydrocarbon stream before it enters the second reaction zone.

278. The method of claim 232, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, wherein the second reaction zone is configured to isomerize at least a portion of linear olefins to branched olefins; and wherein the second reaction zone is operated at a temperature range of about 200° C. to about 500° C.

279. The method of claim 232, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, wherein the second reaction zone is configured to isomerize at least a portion of linear olefins to branched olefins; and wherein the second reaction zone is operated at a hydrocarbon partial pressure between about 0.1 atmosphere and about 10 atmospheres.

280. The method of claim 232, wherein the dehydrogenation-isomerization unit comprises a plurality of zones, wherein the plurality of zones comprises a first reaction zone and a second reaction zone, wherein the first reaction zone is configured to dehydrogenate at least a portion of paraffins to olefins, wherein the second reaction zone is configured to isomerize at least a portion of linear olefins to branched olefins; and wherein the second reaction zone is operated at a hydrocarbon partial pressure between about 0.5 atmosphere and about 2 atmospheres.

281. The method of claim 232, wherein the dehydrogenation-isomerization unit comprises a stacked bed catalyst configuration, wherein the stacked bed catalyst comprises a dehydrogenation catalyst and an isomerization catalyst.
